SEQUENCE LISTING (203 sequences)

SEQ ID NO: 1 - DNA, 25 bp, Artificial Sequence
  Description of Artificial Sequence Synthetic primer
gtgacagtagctataccacgttaca

SEQ ID NO: 2 - DNA, 22 bp, Artificial Sequence
  Description of Artificial Sequence Synthetic primer
agtgttgtacgaaatcccctct

SEQ ID NO: 3 - DNA, 21 bp, Artificial Sequence
  Description of Artificial Sequence Synthetic probe
atggcgatttatctgcatccc

SEQ ID NO: 4 - DNA, 24 bp, Artificial Sequence
  Description of Artificial Sequence Synthetic primer
cgttttgaccatcttcgtctgatt

SEQ ID NO: 5 - DNA, 18 bp, Artificial Sequence
  Description of Artificial Sequence Synthetic primer
tgcgacacgttgcagagt

SEQ ID NO: 6 - DNA, 24 bp, Artificial Sequence
  Description of Artificial Sequence Synthetic primer
cgttttgaccatcttcgtctgatt

SEQ ID NO: 7 - DNA, 18 bp, Artificial Sequence
  Description of Artificial Sequence Synthetic primer
tgcgacacgttgcagcgt

SEQ ID NO: 8 - DNA, 28 bp, Artificial Sequence
  Description of Artificial Sequence Synthetic primer
gatttctcacatatttcagtgcctgatg

SEQ ID NO: 9 - DNA, 23 bp, Artificial Sequence
  Description of Artificial Sequence Synthetic primer
ccagatctgcgattcgctgtaat

SEQ ID NO: 10 - DNA, 17 bp, Artificial Sequence
  Description of Artificial Sequence Synthetic probe
acggacagcagttatac

SEQ ID NO: 11 - DNA, 25 bp, Artificial Sequence
  Description of Artificial Sequence Synthetic primer
gcgaatactggcgagactatttcaa

SEQ ID NO: 12 - DNA, 32 bp, Artificial Sequence
  Description of Artificial Sequence Synthetic primer
gctcatcatagtctttcttattgtatgactca

SEQ ID NO: 13 - DNA, 32 bp, Artificial Sequence
  Description of Artificial Sequence Synthetic primer
gctcgtcatagtctttcttgttgtatgactca

SEQ ID NO: 14 - DNA, 23 bp, Artificial Sequence
  Description of Artificial Sequence Synthetic probe
ccgctcatgcggaaatagccgtt

SEQ ID NO: 15 - DNA, 26 bp, Artificial Sequence
  Description of Artificial Sequence Synthetic primer
tggaaactatcgaacaacatcaaggt

SEQ ID NO: 16 - DNA, 28 bp, Artificial Sequence
  Description of Artificial Sequence Synthetic primer
tgaatacttcatctttggttagccatcc

SEQ ID NO: 17 - DNA, 18 bp, Artificial Sequence
  Description of Artificial Sequence Synthetic probe
attgtacaaggcgatttc

SEQ ID NO: 18 - DNA, 27 bp, Artificial Sequence
  Description of Artificial Sequence Synthetic primer
atgttatttggatgccgttatctccat

SEQ ID NO: 19 - DNA, 27 bp, Artificial Sequence
  Description of Artificial Sequence Synthetic primer
gcttgtaacgtgaaatgtaaaccatca

SEQ ID NO: 20 - DNA, 19 bp, Artificial Sequence
  Description of Artificial Sequence Synthetic probe
ccgaactcatcataaaaag

SEQ ID NO: 21 - DNA, 29 bp, Artificial Sequence
  Description of Artificial Sequence Synthetic primer
gaaataattcctttgatcagggtagaggt

SEQ ID NO: 22 - DNA, 30 bp, Artificial Sequence
  Description of Artificial Sequence Synthetic primer
aatatttgaagaaggaaggcataaggacat

SEQ ID NO: 23 - DNA, 21 bp, Artificial Sequence
  Description of Artificial Sequence Synthetic probe
caggctccaatacccatatgt

SEQ ID NO: 24 - DNA, 28 bp, Artificial Sequence
  Description of Artificial Sequence Synthetic primer
acagagtattgggagaggtttatgagaa

SEQ ID NO: 25 - DNA, 29 bp, Artificial Sequence
  Description of Artificial Sequence Synthetic primer
gaagaaaacctaccaagacaacaattgaa

SEQ ID NO: 26 - DNA, 22 bp, Artificial Sequence
  Description of Artificial Sequence Synthetic probe
ttgaggtttgttctttatttcc

SEQ ID NO: 27 - DNA, 30 bp, Artificial Sequence
  Description of Artificial Sequence Synthetic primer
catgcgttatagaaatgcaattgatagaca

SEQ ID NO: 28 - DNA, 27 bp, Artificial Sequence
  Description of Artificial Sequence Synthetic primer
ccactgaacagatgaataacagaacca

SEQ ID NO: 29 - DNA, 17 bp, Artificial Sequence
  Description of Artificial Sequence Synthetic probe
aacgcgaattattttcg

SEQ ID NO: 30 - DNA, 24 bp, Artificial Sequence
  Description of Artificial Sequence Synthetic primer
ggcactgatgctaaagctgtaaac

SEQ ID NO: 31 - DNA, 25 bp, Artificial Sequence
  Description of Artificial Sequence Synthetic primer
gcaggcctaaaataccttggatcta

SEQ ID NO: 32 - DNA, 16 bp, Artificial Sequence
  Description of Artificial Sequence Synthetic probe
ccgggcgatgtaatta

SEQ ID NO: 33 - DNA, 23 bp, Artificial Sequence
  Description of Artificial Sequence Synthetic primer
cttcctgcaacttgcaacttgaa

SEQ ID NO: 34 - DNA, 23 bp, Artificial Sequence
  Description of Artificial Sequence Synthetic primer
ctcgcctgatcgacaacaaaatg

SEQ ID NO: 35 - DNA, 24 bp, Artificial Sequence
  Description of Artificial Sequence Synthetic probe
agctggcgtaatacttatactcta

SEQ ID NO: 36 - DNA, 29 bp, Artificial Sequence
  Description of Artificial Sequence Synthetic primer
tgacagtatcattacaaagccaaatcagt

SEQ ID NO: 37 - DNA, 21 bp, Artificial Sequence
  Description of Artificial Sequence Synthetic primer
ggccactacgcccttaatctc

SEQ ID NO: 38 - DNA, 22 bp, Artificial Sequence
  Description of Artificial Sequence Synthetic probe
actgaataaagagttaaacgcc

SEQ ID NO: 39 - DNA, 23 bp, Artificial Sequence
  Description of Artificial Sequence Synthetic primer
gtcaagaatgcccgtgaaatagc

SEQ ID NO: 40 - DNA, 21 bp, Artificial Sequence
  Description of Artificial Sequence Synthetic primer
gctcaattctgcatgcatcgg

SEQ ID NO: 41 - DNA, 17 bp, Artificial Sequence
  Description of Artificial Sequence Synthetic probe
taggcaaagcactttcg

SEQ ID NO: 42 - DNA, 26 bp, Artificial Sequence
  Description of Artificial Sequence Synthetic primer
tggaaactatcgaacaacatcaaggt

SEQ ID NO: 43 - DNA, 28 bp, Artificial Sequence
  Description of Artificial Sequence Synthetic primer
tgaatacttcatctttggttagccatcc

SEQ ID NO: 44 - DNA, 18 bp, Artificial Sequence
  Description of Artificial Sequence Synthetic probe
attgtacaaggcgatttc

SEQ ID NO: 45 - DNA, 25 bp, Artificial Sequence
  Description of Artificial Sequence Synthetic primer
gacagatgtgctagtgcaactgata

SEQ ID NO: 46 - DNA, 24 bp, Artificial Sequence
  Description of Artificial Sequence Synthetic primer
gagattttgtccaccacttgcatt

SEQ ID NO: 47 - DNA, 20 bp, Artificial Sequence
  Description of Artificial Sequence Synthetic probe
ctaattgggataacaaaagc

SEQ ID NO: 48 - DNA, 25 bp, Artificial Sequence
  Description of Artificial Sequence Synthetic primer
agtgctggactacagaaaattgcta

SEQ ID NO: 49 - DNA, 25 bp, Artificial Sequence
  Description of Artificial Sequence Synthetic primer
accgaaatgatgggtgctaagaaaa

SEQ ID NO: 50 - DNA, 21 bp, Artificial Sequence
  Description of Artificial Sequence Synthetic probe
ccaccaatagtagcgatagtg

SEQ ID NO: 51 - DNA, 28 bp, Artificial Sequence
  Description of Artificial Sequence Synthetic primer
agagtgataaactcatcttgtcgggtat

SEQ ID NO: 52 - DNA, 22 bp, Artificial Sequence
  Description of Artificial Sequence Synthetic primer
agaatgccactagccaacaaca

SEQ ID NO: 53 - DNA, 18 bp, Artificial Sequence
  Description of Artificial Sequence Synthetic probe
cagaaagtgtgaaatgcc

SEQ ID NO: 54 - DNA, 24 bp, Artificial Sequence
  Description of Artificial Sequence Synthetic primer
tgcgcgtttacatgctgaaaataa

SEQ ID NO: 55 - DNA, 25 bp, Artificial Sequence
  Description of Artificial Sequence Synthetic primer
cgcttactcccaagatgcatactaa

SEQ ID NO: 56 - DNA, 23 bp, Artificial Sequence
  Description of Artificial Sequence Synthetic probe
aacttctccgtatttataaaaat

SEQ ID NO: 57 - DNA, 25 bp, Artificial Sequence
  Description of Artificial Sequence Synthetic primer
ccaggaaacggaattagtccaatgt

SEQ ID NO: 58 - DNA, 27 bp, Artificial Sequence
  Description of Artificial Sequence Synthetic primer
gagtgctcaattagttgatcctcatca

SEQ ID NO: 59 - DNA, 21 bp, Artificial Sequence
  Description of Artificial Sequence Synthetic probe
aatccttttcagctattttac

SEQ ID NO: 60 - DNA, 27 bp, Artificial Sequence
  Description of Artificial Sequence Synthetic primer
atgttatttggatgccgttatctccat

SEQ ID NO: 61 - DNA, 27 bp, Artificial Sequence
  Description of Artificial Sequence Synthetic primer
gcttgtaacgtgaaatgtaaaccatca

SEQ ID NO: 62 - DNA, 19 bp, Artificial Sequence
  Description of Artificial Sequence Synthetic probe
ccgaactcatcataaaaag

SEQ ID NO: 63 - DNA, 25 bp, Artificial Sequence
  Description of Artificial Sequence Synthetic primer
ccgattacagagccttatggatgaa

SEQ ID NO: 64 - DNA, 30 bp, Artificial Sequence
  Description of Artificial Sequence Synthetic primer
gatagccattttccctatataaatgcaggt

SEQ ID NO: 65 - DNA, 18 bp, Artificial Sequence
  Description of Artificial Sequence Synthetic probe
accaagcctagaatgttc

SEQ ID NO: 66 - DNA, 28 bp, Artificial Sequence
  Description of Artificial Sequence Synthetic primer
cgttatcaactttacctgttagagagca

SEQ ID NO: 67 - DNA, 24 bp, Artificial Sequence
  Description of Artificial Sequence Synthetic primer
accagtatcagcatttgagccaat

SEQ ID NO: 68 - DNA, 23 bp, Artificial Sequence
  Description of Artificial Sequence Synthetic probe
ttagcgaagaatacgattatatc

SEQ ID NO: 69 - DNA, 25 bp, Artificial Sequence
  Description of Artificial Sequence Synthetic primer
acgtttgatagattcgcggtagaaa

SEQ ID NO: 70 - DNA, 32 bp, Artificial Sequence
  Description of Artificial Sequence Synthetic primer
agtatccaaaaatgattgaatagctgaagtca

SEQ ID NO: 71 - DNA, 16 bp, Artificial Sequence
  Description of Artificial Sequence Synthetic probe
tcgggcctagaagttt

SEQ ID NO: 72 - DNA, 24 bp, Artificial Sequence
  Description of Artificial Sequence Synthetic primer
gctgtatcagagctcgctatcttt

SEQ ID NO: 73 - DNA, 33 bp, Artificial Sequence
  Description of Artificial Sequence Synthetic primer
gtaatcattagcctgtgagattatatgctctaa

SEQ ID NO: 74 - DNA, 18 bp, Artificial Sequence
  Description of Artificial Sequence Synthetic probe
aattcaagctcaatttcg

SEQ ID NO: 75 - DNA, 25 bp, Artificial Sequence
  Description of Artificial Sequence Synthetic primer
tttggcgaaagttgcttctaaaaca

SEQ ID NO: 76 - DNA, 24 bp, Artificial Sequence
  Description of Artificial Sequence Synthetic primer
agtacggcattaccaaaagaacca

SEQ ID NO: 77 - DNA, 21 bp, Artificial Sequence
  Description of Artificial Sequence Synthetic probe
ccagtgataacgagtgtttta

SEQ ID NO: 78 - DNA, 29 bp, Artificial Sequence
  Description of Artificial Sequence Synthetic primer
gaaataattcctttgatcagggtagaggt

SEQ ID NO: 79 - DNA, 30 bp, Artificial Sequence; identical to SEQ ID NO: 22
  Description of Artificial Sequence Synthetic primer

SEQ ID NO: 80 - DNA, 21 bp, Artificial Sequence
  Description of Artificial Sequence Synthetic probe
caggctccaatacccatatgt

SEQ ID NO: 81 - DNA, 28 bp, Artificial Sequence
  Description of Artificial Sequence Synthetic primer
gggatataaggttcaaagtatcgctgaa

SEQ ID NO: 82 - DNA, 25 bp, Artificial Sequence
  Description of Artificial Sequence Synthetic primer
gacaatccaaccgaaccaaacataa

SEQ ID NO: 83 - DNA, 21 bp, Artificial Sequence
  Description of Artificial Sequence Synthetic probe
ttcatccctttatcaataatg

SEQ ID NO: 84 - DNA, 20 bp, Artificial Sequence
  Description of Artificial Sequence Synthetic primer
cactcttgcttcgcctgttg

SEQ ID NO: 85 - DNA, 26 bp, Artificial Sequence
  Description of Artificial Sequence Synthetic primer
catgttgctttatcaacctcgtgaaa

SEQ ID NO: 86 - DNA, 17 bp, Artificial Sequence
  Description of Artificial Sequence Synthetic probe
tcgcgtacagagacgtt

SEQ ID NO: 87 - DNA, 24 bp, Artificial Sequence
  Description of Artificial Sequence Synthetic primer
ttggagttggcaataattcagggt

SEQ ID NO: 88 - DNA, 27 bp, Artificial Sequence
  Description of Artificial Sequence Synthetic primer
caccaacagcaatacttgaaaagatgt

SEQ ID NO: 89 - DNA, 20 bp, Artificial Sequence
  Description of Artificial Sequence Synthetic probe
cacaggaaatatggcattcg

SEQ ID NO: 90 - DNA, 27 bp, Artificial Sequence
  Description of Artificial Sequence Synthetic primer
ggctcatcaattttgaggctaaatgac

SEQ ID NO: 91 - DNA, 24 bp, Artificial Sequence
  Description of Artificial Sequence Synthetic primer
accctgaattattgccaactccaa

SEQ ID NO: 92 - DNA, 22 bp, Artificial Sequence
  Description of Artificial Sequence Synthetic probe
ttgatgttgaaaaaaaaacttc

SEQ ID NO: 93 - DNA, 23 bp, Artificial Sequence
  Description of Artificial Sequence Synthetic primer
gcaataattcagggtgcgaatgc

SEQ ID NO: 94 - DNA, 29 bp, Artificial Sequence
  Description of Artificial Sequence Synthetic primer
caacagcaatacttgaaaagatgttttcc

SEQ ID NO: 95 - DNA, 16 bp, Artificial Sequence
  Description of Artificial Sequence Synthetic probe
ctgtgttggtattccc

SEQ ID NO: 96 - DNA, 28 bp, Artificial Sequence
  Description of Artificial Sequence Synthetic primer
cccaacaactattggatgatttcttggt

SEQ ID NO: 97 - DNA, 18 bp, Artificial Sequence
  Description of Artificial Sequence Synthetic primer
ccttttaggccaagattc

SEQ ID NO: 98 - DNA, 26 bp, Artificial Sequence
  Description of Artificial Sequence Synthetic probe
gtgaatttgggatattttggcggatt

SEQ ID NO: 99 - DNA, 25 bp, Artificial Sequence
  Description of Artificial Sequence Synthetic primer
acggaattatagtctcgcacatcac

SEQ ID NO: 100 - DNA, 25 bp, Artificial Sequence
  Description of Artificial Sequence Synthetic primer
gtaatgccgtacttaagcgttttct

SEQ ID NO: 101 - DNA, 20 bp, Artificial Sequence
  Description of Artificial Sequence Synthetic probe
ttttcatccctgctaaatat

SEQ ID NO: 102 - DNA, 25 bp, Artificial Sequence
  Description of Artificial Sequence Synthetic primer
gacataacaggctccaatacccata

SEQ ID NO: 103 - DNA, 30 bp, Artificial Sequence
  Description of Artificial Sequence Synthetic primer
gggaagcaggtttttaaattatttgaaagg

SEQ ID NO: 104 - DNA, 18 bp, Artificial Sequence
  Description of Artificial Sequence Synthetic probe
cctgatcaaaggaattat

SEQ ID NO: 105 - DNA, 27 bp, Artificial Sequence
  Description of Artificial Sequence Synthetic primer
gccatcagctatatatgcatgtaacgt

SEQ ID NO: 106 - DNA, 24 bp, Artificial Sequence
  Description of Artificial Sequence Synthetic primer
gcgtgggtaataatccaatttcgt

SEQ ID NO: 107 - DNA, 19 bp, Artificial Sequence
  Description of Artificial Sequence Synthetic probe
aagacagcaccagtccaaa

SEQ ID NO: 108 - DNA, 24 bp, Artificial Sequence
  Description of Artificial Sequence Synthetic primer
catgtaaagcagcaaaccaaatgc

SEQ ID NO: 109 - DNA, 24 bp, Artificial Sequence
  Description of Artificial Sequence Synthetic primer
gcggtacccatgaagtcataattg

SEQ ID NO: 110 - DNA, 17 bp, Artificial Sequence
  Description of Artificial Sequence Synthetic probe
cttgctgggtttattcc

SEQ ID NO: 111 - DNA, 20 bp, Artificial Sequence
  Description of Artificial Sequence Synthetic primer
agcgtcctcaggtcgatagt

SEQ ID NO: 112 - DNA, 23 bp, Artificial Sequence
  Description of Artificial Sequence Synthetic primer
ccacagtttgtcgatagcacatc

SEQ ID NO: 113 - DNA, 22 bp, Artificial Sequence
  Description of Artificial Sequence Synthetic probe
tagcttttttgagagaatggtc

SEQ ID NO: 114 - DNA, 25 bp, Artificial Sequence
  Description of Artificial Sequence Synthetic primer
tctttccccgcaatttattcagtct

SEQ ID NO: 115 - DNA, 24 bp, Artificial Sequence
  Description of Artificial Sequence Synthetic primer
gcgctgcaattgcttatgtatttg

SEQ ID NO: 116 - DNA, 17 bp, Artificial Sequence
  Description of Artificial Sequence Synthetic probe
actgatagtgcaaaatc

SEQ ID NO: 117 - DNA, 24 bp, Artificial Sequence
  Description of Artificial Sequence Synthetic primer
cgggtcataactaccaacgtatcc

SEQ ID NO: 118 - DNA, 24 bp, Artificial Sequence
  Description of Artificial Sequence Synthetic primer
cgtccaaagttactgacaggctat

SEQ ID NO: 119 - DNA, 17 bp, Artificial Sequence
  Description of Artificial Sequence Synthetic probe
acaacacagtgaactcc

SEQ ID NO: 120 - DNA, 28 bp, Artificial Sequence
  Description of Artificial Sequence Synthetic primer
catttagcctcaaaattgatgagccttt

SEQ ID NO: 121 - DNA, 25 bp, Artificial Sequence
  Description of Artificial Sequence Synthetic primer
cagaaggttgcataaaatgggatga

SEQ ID NO: 122 - DNA, 21 bp, Artificial Sequence
  Description of Artificial Sequence Synthetic probe
ttgctatcggtttatttggcc

SEQ ID NO: 123 - DNA, 27 bp, Artificial Sequence
  Description of Artificial Sequence Synthetic primer
ggacaatgaaaaccatgaactttagct

SEQ ID NO: 124 - DNA, 26 bp, Artificial Sequence
  Description of Artificial Sequence Synthetic primer
cacgaggttgataaagcaacatgaat

SEQ ID NO: 125 - DNA, 19 bp, Artificial Sequence
  Description of Artificial Sequence Synthetic probe
cttgcatatagcaggcttt

SEQ ID NO: 126 - DNA, 28 bp, Artificial Sequence
  Description of Artificial Sequence Synthetic primer
acagagtattgggagaggtttatgagaa

SEQ ID NO: 127 - DNA, 29 bp, Artificial Sequence
  Description of Artificial Sequence Synthetic primer
gaagaaaacctaccaagacaacaattgaa

SEQ ID NO: 128 - DNA, 22 bp, Artificial Sequence
  Description of Artificial Sequence Synthetic probe
ttgaggtttgttctttatttcc

SEQ ID NO: 129 - DNA, 27 bp, Artificial Sequence
  Description of Artificial Sequence Synthetic primer
gtttatttccttatgcgattgcaatgc

SEQ ID NO: 130 - DNA, 25 bp, Artificial Sequence
  Description of Artificial Sequence Synthetic primer
aaacatgagaatggctccctgtaaa

SEQ ID NO: 131 - DNA, 22 bp, Artificial Sequence
  Description of Artificial Sequence Synthetic probe
cagtggcttttataatttattc

SEQ ID NO: 132 - DNA, 26 bp, Artificial Sequence
  Description of Artificial Sequence Synthetic primer
cctgtggcttttgttgatgataatcg

SEQ ID NO: 133 - DNA, 25 bp, Artificial Sequence
  Description of Artificial Sequence Synthetic primer
caaccaagctaatttttcgggtgaa

SEQ ID NO: 134 - DNA, 19 bp, Artificial Sequence
  Description of Artificial Sequence Synthetic probe
catggatgacctgcccctg

SEQ ID NO: 135 - DNA, 26 bp, Artificial Sequence
  Description of Artificial Sequence Synthetic primer
ggatggtattaatggtcttgcaagtg

SEQ ID NO: 136 - DNA, 28 bp, Artificial Sequence
  Description of Artificial Sequence Synthetic primer
gtcattgcatcagatgaatcagagtagt

SEQ ID NO: 137 - DNA, 24 bp, Artificial Sequence
  Description of Artificial Sequence Synthetic probe
tcgctttgtagtatcttgataata

SEQ ID NO: 138 - DNA, 23 bp, Artificial Sequence
  Description of Artificial Sequence Synthetic primer
ctaagggtgaggatgttgaggtt

SEQ ID NO: 139 - DNA, 25 bp, Artificial Sequence
  Description of Artificial Sequence Synthetic primer
gcagcatagatgacaattcccagat

SEQ ID NO: 140 - DNA, 15 bp, Artificial Sequence
  Description of Artificial Sequence Synthetic probe
ccaccgcgtgatgat

SEQ ID NO: 141 - DNA, 25 bp, Artificial Sequence
  Description of Artificial Sequence Synthetic primer
gcagttggcaatattctctcgtcat

SEQ ID NO: 142 - DNA, 28 bp, Artificial Sequence
  Description of Artificial Sequence Synthetic primer
gttagatggtgcatccagtaatttagga

SEQ ID NO: 143 - DNA, 16 bp, Artificial Sequence
  Description of Artificial Sequence Synthetic probe
tcggctccacagatcc

SEQ ID NO: 144 - DNA, 18 bp, Artificial Sequence
  Description of Artificial Sequence Synthetic primer
ctggctcgcgtgtgtttt

SEQ ID NO: 145 - DNA, 23 bp, Artificial Sequence
  Description of Artificial Sequence Synthetic primer
acaaaggcagccaatactaacgt

SEQ ID NO: 146 - DNA, 25 bp, Artificial Sequence
  Description of Artificial Sequence Synthetic probe
ttgaaatttttaatagcaaataccc

SEQ ID NO: 147 - DNA, 18 bp, Artificial Sequence
  Description of Artificial Sequence Synthetic primer
ctggctcgcgtgtgtttt

SEQ ID NO: 148 - DNA, 23 bp, Artificial Sequence
  Description of Artificial Sequence Synthetic primer
acaaaggcagccaatactaacgt

SEQ ID NO: 149 - DNA, 25 bp, Artificial Sequence
  Description of Artificial Sequence Synthetic probe
actgctattcattaaatgacaagac

SEQ ID NO: 150 - DNA, 30 bp, Artificial Sequence; identical to SEQ ID NO: 27
  Description of Artificial Sequence Synthetic primer

SEQ ID NO: 151 - DNA, 27 bp, Artificial Sequence
  Description of Artificial Sequence Synthetic primer
ccactgaacagatgaataacagaacca

SEQ ID NO: 152 - DNA, 17 bp, Artificial Sequence
  Description of Artificial Sequence Synthetic probe
aacgcgaattattttcg

SEQ ID NO: 153 - DNA, 30 bp, Artificial Sequence; identical to SEQ ID NO: 27
  Description of Artificial Sequence Synthetic primer

SEQ ID NO: 154 - DNA, 27 bp, Artificial Sequence
  Description of Artificial Sequence Synthetic primer
ccactgaacagatgaataacagaacca

SEQ ID NO: 155 - DNA, 26 bp, Artificial Sequence
  Description of Artificial Sequence Synthetic probe
taaatatattaacgcgaattattttc

SEQ ID NO: 156 - DNA, 24 bp, Artificial Sequence
  Description of Artificial Sequence Synthetic primer
ggcactgatgctaaagctgtaaac

SEQ ID NO: 157 - DNA, 25 bp, Artificial Sequence
  Description of Artificial Sequence Synthetic primer
gcaggcctaaaataccttggatcta

SEQ ID NO: 158 - DNA, 16 bp, Artificial Sequence
  Description of Artificial Sequence Synthetic probe
ccgggcgatgtaatta

SEQ ID NO: 159 - DNA, 26 bp, Artificial Sequence
  Description of Artificial Sequence Synthetic primer
aacatcgtgaatacctttggctaact

SEQ ID NO: 160 - DNA, 25 bp, Artificial Sequence
  Description of Artificial Sequence Synthetic primer
cctccggcaataattacagggtaaa

SEQ ID NO: 161 - DNA, 20 bp, Artificial Sequence
  Description of Artificial Sequence Synthetic probe
ttctctcgcatattaatttt

SEQ ID NO: 162 - DNA, 25 bp, Artificial Sequence
  Description of Artificial Sequence Synthetic primer
ggcgatggcgcattaagaaataaat

SEQ ID NO: 163 - DNA, 29 bp, Artificial Sequence
  Description of Artificial Sequence Synthetic primer
ttctttaatatcacttctttgccccaaga

SEQ ID NO: 164 - DNA, 21 bp, Artificial Sequence
  Description of Artificial Sequence Synthetic probe
aatcttgtggataaagttttc

SEQ ID NO: 165 - DNA, 24 bp, Artificial Sequence
  Description of Artificial Sequence Synthetic primer
acgaaggtcccgataggaacatat

SEQ ID NO: 166 - DNA, 25 bp, Artificial Sequence
  Description of Artificial Sequence Synthetic primer
cgctcataatgggcaatggtaaatt

SEQ ID NO: 167 - DNA, 17 bp, Artificial Sequence
  Description of Artificial Sequence Synthetic probe
caggcagtgaatggtac

SEQ ID NO: 168 - DNA, 25 bp, Artificial Sequence
  Description of Artificial Sequence Synthetic primer
gctgattggtttctgagatgtttca

SEQ ID NO: 169 - DNA, 23 bp, Artificial Sequence
  Description of Artificial Sequence Synthetic primer
gaaaccccaccatatcccattct

SEQ ID NO: 170 - DNA, 23 bp, Artificial Sequence
  Description of Artificial Sequence Synthetic probe
ttaatgacacgacccctattgtt

SEQ ID NO: 171 - DNA, 25 bp, Artificial Sequence
  Description of Artificial Sequence Synthetic primer
cacctgagttcataatgggaacaag

SEQ ID NO: 172 - DNA, 29 bp, Artificial Sequence
  Description of Artificial Sequence Synthetic primer
tatttacttgcactatcatgctctaatat

SEQ ID NO: 173 - DNA, 20 bp, Artificial Sequence
  Description of Artificial Sequence Synthetic probe
taccaaccatgccagttgag

SEQ ID NO: 174 - DNA, 25 bp, Artificial Sequence
  Description of Artificial Sequence Synthetic primer
cttaaaatatcgcgggcttcgaaat

SEQ ID NO: 175 - DNA, 22 bp, Artificial Sequence
  Description of Artificial Sequence Synthetic primer
tgccgctgagatatcctaagct

SEQ ID NO: 176 - DNA, 23 bp, Artificial Sequence
  Description of Artificial Sequence Synthetic probe
ctagcctctgaaaatacatttat

SEQ ID NO: 177 - DNA, 19 bp, Artificial Sequence
  Description of Artificial Sequence Synthetic primer
gctgcagtccagggtactc

SEQ ID NO: 178 - DNA, 25 bp, Artificial Sequence
  Description of Artificial Sequence Synthetic primer
ccgttccttaactcaaaatggcaat

SEQ ID NO: 179 - DNA, 20 bp, Artificial Sequence
  Description of Artificial Sequence Synthetic probe
cccagcattaactaattcag

SEQ ID NO: 180 - DNA, 25 bp, Artificial Sequence
  Description of Artificial Sequence Synthetic primer
cgagaagagatgtgtaacacgcaat

SEQ ID NO: 181 - DNA, 25 bp, Artificial Sequence
  Description of Artificial Sequence Synthetic primer
gtttggacaatccattggtggaatt

SEQ ID NO: 182 - DNA, 17 bp, Artificial Sequence
  Description of Artificial Sequence Synthetic probe
ccactgatcgaaccttt

SEQ ID NO: 183 - DNA, 25 bp, Artificial Sequence
  Description of Artificial Sequence Synthetic primer
gttgtttctcagcgctgtatgtatc

SEQ ID NO: 184 - DNA, 25 bp, Artificial Sequence
  Description of Artificial Sequence Synthetic primer
cgcagtgaaaactctggagtgatta

SEQ ID NO: 185 - DNA, 21 bp, Artificial Sequence
  Description of Artificial Sequence Synthetic probe
atcttcatccacttattatcg

SEQ ID NO: 186 - DNA, 26 bp, Artificial Sequence
  Description of Artificial Sequence Synthetic primer
gtatggcgttgtttttctcaatcaga

SEQ ID NO: 187 - DNA, 28 bp, Artificial Sequence
  Description of Artificial Sequence Synthetic primer
ccaagaaacagacttcattaggaagagt

SEQ ID NO: 188 - DNA, 18 bp, Artificial Sequence
  Description of Artificial Sequence Synthetic probe
acggcggtagttgttaat

SEQ ID NO: 189 - DNA, 25 bp, Artificial Sequence
  Description of Artificial Sequence Synthetic primer
tcccttttggtggttctgttagatc

SEQ ID NO: 190 - DNA, 31 bp, Artificial Sequence
  Description of Artificial Sequence Synthetic primer
aggaagtaatactattttgcgtggtaaaaca

SEQ ID NO: 191 - DNA, 19 bp, Artificial Sequence
  Description of Artificial Sequence Synthetic probe
caaaccctacaccattatc

SEQ ID NO: 192 - DNA, 31 bp, Artificial Sequence
  Description of Artificial Sequence Synthetic primer
actaagtcactgctagaaagatatacaacca

SEQ ID NO: 193 - DNA, 27 bp, Artificial Sequence
  Description of Artificial Sequence Synthetic primer
ggaaggaacaattacgttgaaatgtga

SEQ ID NO: 194 - DNA, 22 bp, Artificial Sequence
  Description of Artificial Sequence Synthetic probe
ccgcgatgcttgttttttgtcg

SEQ ID NO: 195 - DNA, 20 bp, Artificial Sequence
  Description of Artificial Sequence Synthetic primer
ctgttcgccgcaatctttcc

SEQ ID NO: 196 - DNA, 24 bp, Artificial Sequence
  Description of Artificial Sequence Synthetic primer
cctgtaagcctgaaaacaccgtta

SEQ ID NO: 197 - DNA, 22 bp, Artificial Sequence
  Description of Artificial Sequence Synthetic probe
aaggcgatatcatctactttta

SEQ ID NO: 198 - DNA, 21 bp, Artificial Sequence
  Description of Artificial Sequence Synthetic primer
gtaaatgtatcccccgccagt

SEQ ID NO: 199 - DNA, 25 bp, Artificial Sequence
  Description of Artificial Sequence Synthetic primer
tggttgggtatttgtaaggcattga

SEQ ID NO: 200 - DNA, 18 bp, Artificial Sequence
  Description of Artificial Sequence Synthetic probe
ttgctggtgagataattc

SEQ ID NO: 201 - DNA, 27 bp, Artificial Sequence
  Description of Artificial Sequence Synthetic primer
tgcaatcacggtatctgaaataaacca

SEQ ID NO: 202 - DNA, 23 bp, Artificial Sequence
  Description of Artificial Sequence Synthetic primer
gcgcaggcagtattaccttatgt

SEQ ID NO: 203 - DNA, 18 bp, Artificial Sequence
  Description of Artificial Sequence Synthetic probe
tagccagaggcacacctg

(19) United States
(12) Patent Application Publication
(10) Pub. No.: US 2014/0005061 A1
(43) Pub. Date: Jan. 2, 2014

(54) COMPOSITIONS AND METHODS FOR DETECTION OF MULTIPLE MICROORGANISMS

(71) Applicant: LIFE TECHNOLOGIES CORPORATION, Carlsbad, CA (US)

(72) Inventors: Robert TEBBS, Austin, TX (US); Craig Cummings, Pacifica, CA (US); Pius Brzoska, Woodside, CA (US); Sharon Matheny, Keller, TX (US); Catherine O'Connell, Austin, TX (US); Manohar Furtado, San Ramon, CA (US); Nikolay Sergeev, Foster City, CA (US); Maxim Brevnov, Union City, CA (US)

(73) Assignee: LIFE TECHNOLOGIES CORPORATION, Carlsbad, CA (US)

(21) Appl. No.: 13/790,447

(22) Filed: Mar. 8, 2013

Related U.S. Application Data
(60) Provisional application No. 61/666,325, filed on Jun. 29, 2012.

(51) Int. Cl. C12Q 1/68

(52) U.S. Cl. CPC C12Q 1/689
USPC 506/9; 536/24.33; 536/24.32; 435/6.12; 435/6.11

(57) ABSTRACT

The present disclosure describes compositions, methods and kits for detection of one or multiple microorganism contaminants in samples. Some embodiments relate to detecting one or more microorganisms producing virulence factors such as shiga toxin stx1 and stx2 and eae. Some embodiments relate to detection of STEC microorganisms including an E. coli O26, an E. coli O45, an E. coli O103, an E. coli O111, an E. coli O121 or an E. coli O145. In some embodiments, compositions, methods and kits can detect and identify individual serotypes of shiga toxin producing microorganisms. Workflows for multiple microbe detection and identification are also described.

### FIELD

The present disclosure describes compositions, methods and kits for detection of one or multiple microorganism contaminants in samples. Some embodiments describe compositions, methods and kits for detecting one or more microorganisms producing virulence factors such as shiga toxin or eae. In some embodiments, compositions, methods and kits can further identify and detect individual serotypes of shiga toxin-producing microorganisms. Workflows for multiple microbe detection and identification are also described.

### BACKGROUND

Identification of bacterial contamination in food often occurs subsequent to an outbreak of a foodborne illness. The bacterium Escherichia coli (E. coli) is frequently identified as a food contaminant of many foodborne illnesses. Some E. coli organisms produce a toxin called shiga toxin.

Shiga toxin-producing E. coli (STEC), which are also sometimes known as verotoxin-producing E. coli (VTEC), are found in undercooked meats, raw milk, and contaminated water, to name a few examples. These bacteria can cause illness ranging from mild intestinal diseases to bloody diarrhea to life-threatening kidney failure caused by a condition called Hemolytic Uremic Syndrome (HUS).

An E. coli serotype known as E. coli O157:H7 produces shiga toxin and is most often associated with outbreaks of foodborne illness in the United States and elsewhere in the world and causes enterohemorrhagic colitis and possibly kidney failure. Detection of pathogenic E. coli, particularly serotypes causative of hemorrhagic colitis and HUS has become a public health priority. Regulations by the United States government require meat and other food processors to screen for the presence of strains such as O157:H7 in their finished products and more stringent guidelines are being considered in a number of states for the identification of O157:H7 in other commodities and foodstuffs.

As a result of repeated outbreaks and the life-threatening nature of STEC contamination, government public health agencies are now requiring testing of multiple STEC microbes to ensure food safety. Specifically, the USDA-FSIS has identified six O-serotypes of E. coli including O26, O45, O103, O111, O121, O145 for future testing while the European Food Safety Association (EFSA) has required testing of four of these E. coli serotypes including O26, O103, O111, O145. However, rapid methods for testing and identifying one or more STEC organisms are still lacking.

Assays for the rapid, sensitive, and specific detection of infectious pathogens are extremely important from both a public health and economic perspective. There exists a need for novel assays and methods for detecting and differentiating pathogenic organisms from other pathogenic and non-pathogenic organisms to test food and other samples, and identification of pathogens for example to determine a pathogenic contaminant in a sample and/or to make a differential diagnosis of what microbe is causing a particular disease.

### SUMMARY

The present disclosure, in some embodiments, describes compositions, kits and methods of use thereof for detection of microorganisms and strains expressing a virulence factor (e.g., a STEC microbe that express virulence factors such as a shiga toxin and/or an eae).

Some embodiments relate to compositions comprising isolated nucleic acid sequences that are operable to hybridize to nucleic acid regions that are uniquely found in a STEC microorganism. Nucleic acid regions uniquely found in a STEC microorganism specific are referred to herein as microorganism-specific target nucleic acids or STEC microorganism-specific target nucleic acids.

Some embodiments relate to compositions comprising isolated nucleic acid sequences that are operable to hybridize to nucleic acid regions corresponding to virulence factors expressed in a microorganism. Nucleic acid regions corresponding to virulence factors are referred to herein as virulence factor-specific target nucleic acids.

In some embodiments, the present disclosure describes isolated nucleic acid sequence comprising SEQ ID NO:15-SEQ ID NO:32 and SEQ ID NO: 39-SEQ ID NO: 203, fragments thereof, complements thereof, sequences comprising at least 90% nucleic acid sequence identity thereto, labeled derivatives, and chemical and/or biological derivatives thereof. In some embodiments, the present disclosure describes isolated nucleic acid sequence comprising and SEQ ID NO: 1-SEQ ID NO: 10 and SEQ ID NO:11-SEQ ID NO:14, fragments thereof, complements thereof, sequences comprising at least 90% nucleic acid sequence identity thereto and labeled derivatives thereof. Fragments of nucleic acid sequences include without limitation nucleic acids having at least 10, at least 15, or at least 20 contiguous nucleic acids of a nucleic acid sequence as described herein.

Nucleic acid sequences of the disclosure, in some embodiments, are primers and/or probes. In some embodiments, primers of the disclosure can be degenerate primers. Primers and probes can be labeled. In some embodiments, isolated nucleic acid sequence compositions of the disclosure may comprise one or more label, such as, but not limited to, a dye, a radioactive isotope, a chemiluminescent label, a fluorescent moiety, a bioluminescent label, an enzyme, and combinations thereof. In some embodiments primers and probes can be chemical and/or biological derivatives of the sequences described here.

The present application describes compositions and methods for detecting multiple microorganisms in samples. One or more microorganism that can be detected by a method of this disclosure are different strains of E. coli such as an E. coli O157:H7, an E. coli O26, an E. coli O45, an E. coli O103, an E. coli O111, an E. coli O121 and an E. coli O145. Some methods disclosed here are singleplex methods and some methods disclosed herein are multiplex methods.

Samples that can be tested by methods of the disclosure to detect a microbial contaminant therein include but are not limited to a food sample, a beverage sample, an agricultural sample, a produce sample, an animal sample, a clinical sample, an environmental sample, a biological sample, a water sample and an air sample.

Some embodiments describe methods for detection of a STEC E. coli strain in a sample comprising: hybridizing a first pair of nucleic acid amplification to a first STEC E. coli strain target polynucleotide sequence present in the sample; amplifying at least a first STEC E. coli strain target polynucleotide sequence or a fragment or a complement thereof to obtain at least one amplified STEC E. coli strain target polynucleotide sequence; and detecting the at least one amplified STEC E. coli strain target polynucleotide sequence; wherein detection of the at least one amplified STEC E. coli strain target polynucleotide sequence is indicative of the presence of the STEC E. coli strain in the sample; and the method optionally further comprising using a probe to detect the at least one amplified the STEC E. coli strain target polynucleotide sequence. More than one primer pairs can be hybridized to detect more than one STEC E. coli strains specific target polynucleotide sequences.

Some embodiments describe methods for detection of an E. coli O121 in a sample comprising: hybridizing a first pair of nucleic acid amplification to a first E. coli O121 target polynucleotide sequence present in the sample; amplifying at least a first E. coli O121 target polynucleotide sequence or a fragment or a complement thereof to obtain at least one amplified E. coli O121 target polynucleotide sequence; and detecting the at least one amplified E. coli O121 target polynucleotide sequence; wherein detection of the at least one amplified E. coli O121 target polynucleotide sequence is indicative of the presence of E. coli O121 in the sample; and the method optionally further comprising using a probe to detect the at least one amplified E. coli O121 target polynucleotide sequence. More than one primer pairs can be hybridized to detect more than one E. coli O121 target polynucleotide sequences. In some example embodiments, this method comprises: hybridizing at least one pair of PCR primers (at least a first primer) comprising nucleic acids of SEQ ID NO: 15 and SEQ ID NO: 16, or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55, complements thereof, labeled derivatives thereof, sequences having at least 90% homology thereto, and fragments having at least 10 contiguous nucleotides thereof, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O121 in the sample. A method to detect E. coli O121 can further comprise using a probe to detect the at least one amplified target polynucleotide sequence wherein the probe comprises a sequence of SEQ ID NO: 17, SEQ ID NO: 41, SEQ ID NO:44, SEQ ID NO:47, SEQ ID NO:50, SEQ ID NO:53, or SEQ ID NO:56, labeled derivatives thereof, fragments having at least 10 contiguous nucleotides thereof, complements thereof, and sequences having at least 90% homology thereto. More than one primer pairs and probes can be hybridized as described in the methods to detect more than one E. coli O121 specific target polynucleotide sequences.

Some embodiments describe methods for detection of an E. coli O145 in a sample comprising: hybridizing a first pair of nucleic acid amplification to a first E. coli O145 target polynucleotide sequence present in the sample; amplifying at least a first E. coli O145 target polynucleotide sequence or a fragment or a complement thereof to obtain at least one amplified E. coli O145 target polynucleotide sequence; and detecting the at least one amplified E. coli O145 target polynucleotide sequence; wherein detection of the at least one amplified E. coli O145 target polynucleotide sequence is indicative of the presence of E. coli O145 in the sample; and the method optionally further comprising using a probe to detect the at least one amplified E. coli O145 target polynucleotide sequence. More than one primer pairs can be hybridized to detect more than one E. coli O145 target polynucleotide sequences. In some example embodiments, a method for detection of an E. coli O145 in a sample comprises: hybridizing at least a first pair of PCR primers comprising nucleic acids of SEQ ID NO: 18 and SEQ ID NO: 19, SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74, labeled derivatives thereof, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O145 in the sample. A method to detect an E. coli O145 can further comprise using a probe to detect the at least one amplified target polynucleotide sequence wherein the probe comprises a sequence of SEQ ID NO: 20, SEQ ID NO: 59, SEQ ID NO:62, SEQ ID NO:65, SEQ ID NO:68, SEQ ID NO:71, or SEQ ID NO:74, fragments having at least 10 contiguous nucleotides thereof, labeled derivatives thereof, complements thereof, and sequences having at least 90% homology thereto. More than one primer pairs and probes can be hybridized as described in the methods to detect more than one E. coli O145 specific target polynucleotide sequences.

Some embodiments describe methods for detection of an E. coli O26 in a sample comprising: hybridizing a first pair of nucleic acid amplification to a first E. coli O26 target polynucleotide sequence present in the sample; amplifying at least a first E. coli O26 target polynucleotide sequence or a fragment or a complement thereof to obtain at least one amplified E. coli O26 target polynucleotide sequence; and detecting the at least one amplified E. coli O26 target polynucleotide sequence; wherein detection of the at least one amplified E. coli O26 target polynucleotide sequence is indicative of the presence of E. coli O26 in the sample; and the method optionally further comprising using a probe to detect the at least one amplified E. coli O26 target polynucleotide sequence. More than one primer pairs can be hybridized to detect more than one E. coli O26 target polynucleotide sequences. In some example embodiments, a method for detection of an E. coli O26 in a sample comprises: hybridizing at least a first pair of PCR primers comprising nucleic acids of SEQ ID NO: 21 and SEQ ID NO: 22, or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124, derivatives thereof, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O26 in the sample. A method to detect an E. coli O26 can further comprise using a probe to detect the at least one amplified target polynucleotide sequence wherein the probe comprises a sequence of SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, or SEQ ID NO: 125, derivatives thereof, fragments having at least 10 contiguous nucleotides thereof, complements thereof, and sequences having at least 90% homology thereto. More than one primer pairs and probes can be hybridized as described in the methods to detect more than one E. coli O26 specific target polynucleotide sequences.

Some embodiments describe methods for detection of an E. coli O45 in a sample comprising: hybridizing a first pair of nucleic acid amplification to a first E. coli O45 target polynucleotide sequence present in the sample; amplifying at least a first E. coli O45 target polynucleotide sequence or a fragment or a complement thereof to obtain at least one amplified E. coli O45 target polynucleotide sequence; and detecting the at least one amplified E. coli O45 target polynucleotide sequence; wherein detection of the at least one amplified E. coli O45 target polynucleotide sequence is indicative of the presence of E. coli O45 in the sample; and the method optionally further comprising using a probe to detect the at least one amplified E. coli O45 target polynucleotide sequence. More than one primer pairs can be hybridized to detect more than one E. coli O45 target polynucleotide sequences. In some example embodiments, a method for detection of an E. coli O45 in a sample comprises: hybridizing at least a first pair of PCR primers comprising nucleic acids of SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142, complements thereof, labeled derivatives thereof, fragments having at least 10 contiguous nucleotides thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O45 in the sample. A method to detect an E. coli O45 can further comprise using a probe to detect the at least one amplified target polynucleotide sequence wherein the probe comprises a sequence of SEQ ID NO: 26, SEQ ID NO: 128, SEQ ID NO: 131, SEQ ID NO: 134, SEQ ID NO: 137, SEQ ID NO: 140, or SEQ ID NO: 143, complements thereof, labeled derivatives thereof, fragments having at least 10 contiguous nucleotides thereof, and sequences having at least 90% homology thereto. More than one primer pairs and probes can be hybridized as described in the methods to detect more than one E. coli O45 specific target polynucleotide sequences.

Some embodiments describe methods for detection of an E. coli O103 in a sample comprising: hybridizing a first pair of nucleic acid amplification to a first E. coli O103 target polynucleotide sequence present in the sample; amplifying at least a first E. coli O103 target polynucleotide sequence or a fragment or a complement thereof to obtain at least one amplified E. coli O103 target polynucleotide sequence; and detecting the at least one amplified E. coli O103 target polynucleotide sequence; wherein detection of the at least one amplified E. coli O103 target polynucleotide sequence is indicative of the presence of E. coli O103 in the sample; and the method optionally further comprising using a probe to detect the at least one amplified E. coli O103 target polynucleotide sequence. More than one primer pairs can be hybridized to detect more than one E. coli O103 target polynucleotide sequences. In some example embodiments, a method for detection of an E. coli O103 in a sample comprises: hybridizing at least a first pair of PCR primers comprising nucleic acids of SEQ ID NO: 27 and SEQ ID NO: 28, or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154, complements thereof, labeled derivatives thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O103 in the sample. A method to detect an E. coli O103 can further comprise using a probe to detect the at least one amplified target polynucleotide sequence wherein the probe comprises a sequence of SEQ ID NO: 29, SEQ ID NO: 146, SEQ ID NO: 149, SEQ ID NO: 152, or SEQ ID NO: 155, complements thereof, labeled derivatives thereof, fragments having at least 10 contiguous nucleotides thereof and sequences having at least 90% homology thereto. More than one primer pairs and probes can be hybridized as described in the methods to detect more than one E. coli O103 specific target polynucleotide sequences.

Some embodiments describe methods for detection of an E. coli O111 in a sample comprising: hybridizing a first pair of nucleic acid amplification to a first E. coli O111 target polynucleotide sequence present in the sample; amplifying at least a first E. coli O111 target polynucleotide sequence or a fragment or a complement thereof to obtain at least one amplified E. coli O111 target polynucleotide sequence; and detecting the at least one amplified E. coli O111 target polynucleotide sequence; wherein detection of the at least one amplified E. coli O111 target polynucleotide sequence is indicative of the presence of E. coli O111 in the sample; and the method optionally further comprising using a probe to detect the at least one amplified E. coli O111 target polynucleotide sequence. More than one primer pairs can be hybridized to detect more than one E. coli O111 target polynucleotide sequences. In some example embodiments, a method for detection of an E. coli O111 in a sample comprises: hybridizing at least a first pair of PCR primers comprising nucleic acids of SEQ ID NO: 30 and SEQ ID NO: 31, or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172, complements thereof, labeled derivatives thereof, fragments having at least 10 contiguous nucleotides thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O111 in the sample. A method to detect an E. coli O111 can further comprise using a probe to detect the at least one amplified target polynucleotide sequence wherein the probe comprises a sequence of SEQ ID NO: 32, SEQ ID NO: 158, SEQ ID NO: 161, SEQ ID NO: 164, SEQ ID NO: 167, SEQ ID NO: 170, or SEQ ID NO: 173, labeled derivatives thereof, fragments having at least 10 contiguous nucleotides thereof, complements thereof, and sequences having at least 90% homology thereto. More than one primer pairs and probes can be hybridized as described in the methods to detect more than one E. coli O111 specific target polynucleotide sequences.

Some embodiments describe methods for detection of an E. coli O157:H7 in a sample comprising: hybridizing a first pair of nucleic acid amplification to a first E. coli O157:H7 target polynucleotide sequence present in the sample; amplifying at least a first E. coli O157:H7 target polynucleotide sequence or a fragment or a complement thereof to obtain at least one amplified E. coli O157:H7 target polynucleotide sequence; and detecting the at least one amplified E. coli O157:H7 target polynucleotide sequence; wherein detection of the at least one amplified E. coli O157:H7 target polynucleotide sequence is indicative of the presence of E. coli O157:H7 in the sample; and the method optionally further comprising using a probe to detect the at least one amplified E. coli O157:H7 target polynucleotide sequence. More than one primer pairs can be hybridized to detect more than one E. coli O157:H7 target polynucleotide sequences. In some example embodiments, a method for detection of an E. coli O157:H7 in a sample comprises: hybridizing at least a first pair of PCR primers comprising nucleic acids of SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202 complements thereof, labeled derivatives thereof, and sequences having at least 90% homology thereto to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O015:H7 in the sample. A method to detect an E. coli O157:H7 can further comprise using a probe to detect the at least one amplified target polynucleotide sequence wherein the probe comprises a sequence of SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200, or SEQ ID NO: 203, complements thereof, labeled derivatives thereof, and sequences having at least 90% homology thereto. More than one of these primer pairs and probes can be hybridized as described in the methods to detect more than one E. coli O157:H7 specific target polynucleotide sequences. In addition, for detection of an E. coli O157:H7 primer and probe sequences operable to bind to and/or amplify and/or detect and/or identify microorganism are also described in SEQ ID NOS: 33-SEQ ID NO: 38 and may be used in place of and/or in addition to the E. coli O157:H7 primers and probes described above. Sequences SEQ ID NOS: 33-SEQ ID NO: 38 and compositions thereof and methods for detection of an E. coli O157:H7 using these sequences are also described in U.S. Provisional Patent Application Ser. No. 61/178,931, filed May 15, 2009 (Attorney Docket NO. LT00032Pro); U.S. patent application Ser. No. 12/780,707 filed May 14, 2010 (Attorney Docket NO. LT00032US); PCT Application Serial No. PCT/US2010/034998, filed May 14, 2010 (Attorney Docket NO. LT00032PCT); and European Patent Application Serial No. 10720105.5, filed May 14, 2010, (Attorney Docket NO. LT00032EP), the entire disclosures of which are incorporated herein by reference.

The specification, in some embodiments, describes method for detection of one or more microorganisms, each microorganism expressing one or more virulence factors comprising: 1) detecting the presence of one or more virulence factors comprising: a) contacting a sample suspected of containing one or more microorganisms expressing one or more virulence factors with one or more sets of primers specific to the one or more virulence factors; b) amplifying at least one target nucleic acid sequence encoding at least one of the virulence factors by a multiplex amplification method to obtain one or more amplified virulence factor specific nucleic acids; c) detecting the one or more amplified virulence factor specific nucleic acids or fragments or complements thereof; and d) optionally identifying the amplified virulence factor specific nucleic acids, wherein detecting the one or more amplified virulence factor specific nucleic acids or fragments or complements thereof is indicative of the presence of a microorganism in the sample expressing the one or more virulence factor; and 2) determining the strain of the one or more microorganisms expressing the one or more virulence factors comprising: a) contacting the sample with at least one set of primers specific to the strain of the one or more microorganism; b) amplifying at least one strain specific target nucleic acid sequence encoding for nucleic acid targets specific to at least one strain of the microorganism by a multiplex amplification method to obtain one or more amplified strain specific nucleic acids; c) detecting the one or more amplified strain-specific nucleic acid or fragments or complements thereof; and d) optionally identifying the amplified strain-specific nucleic acids. In some embodiments of the method each set of primers specific to the one or more virulence factors is labeled with a different label. In some embodiments of the method, each set of primers specific to the one or more strain of the microorganism is labeled with a different label and wherein each set of primers specific to the one or more virulence factors is labeled with a different label.

In some embodiments, steps described in the method above of 1) detecting presence of one or more virulence factors and 2) determining strain of the one or more microorganism expressing the one or more virulence factor can be performed simultaneously or sequentially, consequently and/or in any order.

In some embodiments of a method for detection of one or more microorganisms expressing one or more virulence factors, the one or more sets of primers specific to the one or more virulence factors each comprise: at least one forward primer and at least one reverse primer operable to hybridize to one or more virulence factors. Virulence factors in some embodiments comprise but are not limited to one or more of the following: all variants of shiga toxin (stx) encoding nucleic acids, alleles and fragments thereof; and/or all variants of eae gene encoding nucleic acids, alleles and fragments thereof. A stx-encoding nucleic acid may comprise an stx/gene, a stx2 gene, an allele of an stx1 gene an allele of an stx2 gene, or a fragments thereof. For example, see Table 1 which lists several stx1 and stx2 subtypes and variants. An eae encoding nucleic acids may comprise an eae gene, an allele of an eae gene, or a fragments thereof. There are over 25 defined allele of the eae gene, which encodes the intimin protein. See http://onlinelibrary.wiley.com/doi/10.1111/j.1574-6968.2006.00328.x/full for more details.

Microorganisms expressing one or more virulence factors include for example Shiga toxin-producing E. coli (STEC).

In some embodiments of a method described above, one or more primer sets specific for one or more virulence factors may comprise: a first primer set having SEQ ID NO: 1 and SEQ ID NO 2; a second primer set having SEQ ID NO: 4 and SEQ ID NO 5; a third primer set having SEQ ID NO: 6 and SEQ ID NO:7; a fourth primer set having SEQ ID NO: 8 and SEQ ID NO: 9; and/or a fifth primer set having SEQ ID NO: 11, SEQ ID NO: 12; and SEQ ID NO: 13. A method may further comprise the steps of detecting the one or more amplified virulence factor specific nucleic acids or fragments or complements thereof comprises hybridizing the one or more amplified virulence factor specific nucleic acids or fragments or complements thereof with a probe selected from SEQ ID NO: 3, SEQ ID NO: 10, and SEQ ID NO: 14.

In some embodiments, not detecting any amplified product using the methods described above may be used to exclude the presence of a microorganism expressing a virulence factor in a sample.

In some embodiments, either following or simultaneously with the detection of expression of a virulence factor, a method can comprise detecting the strain of a contaminating microorganism. In some embodiments the one or more microorganism strains that can be detected comprise an E. coli O157:H7, an E. coli O26, an E. coli O45, an E. coli O103, an E. coli O111, an E. coli O121 or an E. coli O145. An example method can comprise detection of one or more microorganisms expressing at least one virulence factor comprising: 1) detecting the presence of one or more virulence factors comprising: a) contacting a sample suspected of containing one or more microorganisms expressing at least one virulence factors with one or more sets of primers specific to the one or more virulence factors; b) amplifying at least one target nucleic acid sequence encoding at least one of the virulence factors to obtain one or more amplified virulence factor specific nucleic acids; c) detecting the one or more amplified virulence factor specific nucleic acids or fragments or complements thereof; and d) optionally identifying the amplified virulence factor specific nucleic acids, wherein detecting the one or more amplified virulence factor specific nucleic acids or fragments or complements thereof is indicative of the presence of a microorganism in the sample expressing the at least one virulence factor; and 2) determining strain of the one or more microorganism expressing the at least one virulence factor comprising: a) contacting the sample with at least one set of primers specific to a strain of the one or more microorganism; b) amplifying at least one strain specific target nucleic acid sequence encoding for nucleic acid targets specific to the at least one strain of the microorganism to obtain one or more amplified strain specific nucleic acids; c) detecting the one or more amplified strain specific nucleic acid or fragments or complements thereof; and d) optionally identifying the amplified strain specific nucleic acids.

In one example the step of 2) determining strain of the one or more microorganism expressing the at least one virulence factor can comprise: a) contacting the sample with at least one set of primers specific to a strain of the one or more microorganism comprising selecting at least one set of primers specific to one or more microorganism strains from primer sets as follows: wherein a first primer set comprises one or more primers selected from SEQ ID NO: 15 and SEQ ID NO 16; or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55; wherein a second primer set comprises one or more primers selected from SEQ ID NO: 18 and SEQ ID NO 19; SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74; wherein a third primer set comprises one or more primers selected from SEQ ID NO: 21 and SEQ ID NO: 22; or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124; wherein a fourth primer set comprises one or more primers selected from SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142; wherein a fifth primer set comprises one or more primers selected from SEQ ID NO: 27, SEQ ID NO: 28; or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154; wherein a sixth primer set comprises one or more primers selected from SEQ ID NO: 30 and SEQ ID NO: 31; or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172; and wherein a seventh primer set comprises one or more primers selected from SEQ ID NO: 33 and SEQ ID NO 34 or SEQ ID NO: 36 and SEQ ID NO: 37, or SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202. A method can comprise contacting with one or more of these primer sets from each primer set described above and also from among the first, second, . . . seventh primer sets. In some embodiments, the one or more microorganism strains that are detectable by this method are an E. coli O157:H7, an E. coli O26, an E. coli O45, an E. coli O103, an E. coli O111, an E. coli O121 or an E. coli O145.

In some embodiments of a method as described above, the at least one set of primers specific to the strain of the one or more microorganism can comprise a first primer set having SEQ ID NO: 15 and SEQ ID NO 16; a second primer set having SEQ ID NO: 18 and SEQ ID NO 19; a third primer set having SEQ ID NO: 21 and SEQ ID NO: 22; a fourth primer set having SEQ ID NO: 24 and SEQ ID NO: 25, a fifth primer set having SEQ ID NO: 27, SEQ ID NO: 28; a sixth primer set having SEQ ID NO: 30 and SEQ ID NO: 31; a seventh primer set having SEQ ID NO: 33 and SEQ ID NO 34; and/or an eighth primer set having SEQ ID NO: 36 and SEQ ID NO: 37. In some embodiments, the steps of detecting the one or more amplified strain specific nucleic acids or fragments or complements thereof comprises hybridizing the one or more amplified strain specific nucleic acids or fragments or complements thereof with a probe selected from SEQ ID NO: 17, SEQ ID NO: 20, SEQ ID NO: 23, SEQ ID NO: 26, SEQ ID NO: 29, SEQ ID NO: 32, SEQ ID NO: 35, and SEQ ID NO: 38. Other combinations and variations are also contemplated.

In some embodiments, detecting an amplified nucleic acid comprises one or more methods such as but not limited to hybridization, mass spectrometry, nanostring, microfluidics, chemiluminescence, enzyme technologies and combinations thereof. Some embodiments may also comprise identifying a microbe and may comprise methods such as DNA sequencing.

In some embodiments a method according to the disclosure can further comprise steps of: immunomagnetic separation of desired microorganisms from other sample components including other microbes in the sample; and also optionally enrichment of the separated microorganisms. Immunomagnetic separation comprises using magnetic beads having antibodies specific to one or more microorganisms that are desired to be detected. Immunomagnetic beads are operable to bind to and/or associate with and/or form complexes with certain microorganisms via the antibodies, thereby forming complexes of bead-microbe to be detected which may then be separated from other microbes and other sample components. Immunomagnetic beads of the disclosure may have one or more microbe-specific and strain-specific antibodies on its surface. Some immunomagnetic beads of this disclosure may have antibodies specific to bind two or more different types of microbes and/or microbial strains. Some immunomagnetic beads have be able to bind up to seven different microbes and/or microbial strains on a single bead. Some immunomagnetic beads of the disclosure may be able to bind more than seven different microbes and/or microbial strains.

In some embodiments magnetic beads used for immunomagnetic separation are PCR-compatible beads and beads with microorganisms attached can be directly used for PCR without the need for prior RNA/DNA extraction. In some embodiments there can be no immunomagnetic separation but sample nucleic acids (RNA/DNA) can be lysed and/or extracted and/or purified prior to PCR by other methods of nucleic acid lysis, extraction and/or purification.

In some embodiments, the disclosure describes methods for detecting and identifying one or more Shiga toxin-producing E. coli (STEC) microorganisms in a sample comprising the steps of: a) immunomagnetic separation of STEC microorganisms present in the sample from other sample components and other microbes in the sample to obtain separated STEC microorganisms; b) optionally enriching the separated STEC microorganisms; c) performing a multiplex polymerase chain reaction (PCR) on the separated STEC microorganisms to amplify and detect the presence of at least one nucleic acids selected from: nucleic acids encoding for a shiga toxin gene, including nucleic acids encoding stx1 and stx2; nucleic acids encoding an eae gene; nucleic acids specific for an E. coli strain O157:H7, wherein the detection of at least one nucleic acid above indicates the presence of an STEC microorganism or E. coli O157:H7 or both; and d) if a nucleic acid is detected in step c) performing a second round of multiplex PCR with primers specific to amplify and detect the presence one or more nucleic acids encoding for target regions associated with one or more STEC microorganisms including an E. coli O157:H7, an E. coli O26, an E. coli O45, an E. coli O103, an E. coli O111, an E. coli O121 and an E. coli O145.

In other embodiments, not detecting any amplified product using the methods described above may be used to exclude the presence of a STEC microorganism in a sample.

Amplification of target nucleic acids of targets including stx1, stx2, eae, E. coli O157:H7, an E. coli O26, an E. coli O45, an E. coli O103, an E. coli O111, an E. coli O121 and an E. coli O145 can be detected by using a plurality of labels on each different set of primers and/or probes. For example, in step c) above primers and/or probes with a different label for each of stx/, sxt2, eae and E. coli O157:H7 may be used and in step d) primers and/or a probes with a different label each may be used for the various microorganisms.

Magnetic beads used in immunomagnetic separation have one or more antibodies on them comprising antibodies operable to bind to an E. coli O157:H7, an E. coli O26, an E. coli O45, an E. coli O103, an E. coli O111, an E. coli O121, and an E. coli O145.

In some embodiments, methods of the disclosure can be performed on an automated system. Automation decreases the time as well as efficiency and allows processing multiples samples. Automated systems comprise a magnetic separation platform such as but not limited to MagMAX™ Express-96 Magnetic Particle Processor by Life Technologies Corporation; Pathatrix system (http://www.matrixmsci.com/pathatrix.htm).

Detection in the methods above may be performed by a variety of methods, such as but not limited to, by a nucleic acid amplification reaction, the amplification reaction is an end-point determination, the amplification reaction is quantitative, the quantification is a real-time PCR, the real-time PCR is a SYBR® Green Assay or the real-time PCR is a TaqMan® Assay. Detection may in some embodiments be performed by hybridization using probes specific to amplified nucleic acid sequences encoding a target sequence. Combinations of amplification and hybridization may be used for detection according to some embodiments.

In some embodiments, hybridization can comprise at least a first probe and a second probe, the first probe further comprising a first label and said second probe further comprising a second label, wherein both labels are selected from a dye, a radioactive isotope, a chemiluminescent label, and an enzyme, the dye comprises a fluorescein dye, a rhodamine dye, or a cyanine dye, the dye is a fluorescein dye and first probe is labeled with FAM™ dye and said second probe is labeled with VIC® dye.

Some embodiments describe kits for detection of a Shiga toxin-producing E. coli (STEC) microorganism comprising: one or more pairs of forward and reverse polymerase chain reaction (PCR) primers selected from a first primer set comprising one or more primers selected from SEQ ID NO: 15 and SEQ ID NO 16, or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55; a second primer set comprising one or more primers selected from SEQ ID NO: 18 and SEQ ID NO 19, SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74; a third primer set comprising one or more primers selected from SEQ ID NO:21 and SEQ ID NO:22, or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124; a fourth primer set comprising one or more primers selected from SEQ ID NO: 24 and SEQ ID NO 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142; a fifth primer set comprising one or more primers selected from SEQ ID NO:27 and SEQ ID NO:28, or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154; a sixth primer set comprising one or more primers selected from SEQ ID NO: 30 and SEQ ID NO: 31; or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172; and a seventh primer set comprising one or more primers selected from SEQ ID NO: 33 and SEQ ID NO 34 or SEQ ID NO: 36 and SEQ ID NO: 37, or SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202, or sequences complementary to any of these primers, or sequences comprising at least 90% nucleic acid sequence identity thereto, fragments having at least 10 contiguous nucleotides thereof, or a labeled derivative thereof. In some embodiments, a kit of the disclosure can further comprise one or more pairs of forward and reverse PCR primers further selected from an eighth primer set having SEQ ID NO: 11 and SEQ ID NO: 12 and SEQ ID NO: 13; a ninth primer set having SEQ ID NO: 1 and SEQ ID NO: 2; a tenth primer set having SEQ ID NO:4 and SEQ ID NO:5; an eleventh primer set having SEQ ID NO: 6 and SEQ ID NO: 7; a twelfth primer set having SEQ ID NO:8 and SEQ ID NO:9. A kit can further comprise one more probe selected from probes having SEQ ID NO: 3, SEQ ID NO: 10, SEQ ID NO: 14, SEQ ID NO: 17, SEQ ID NO: 41, SEQ ID NO:44, SEQ ID NO:47, SEQ ID NO:50, SEQ ID NO:53, SEQ ID NO:56, SEQ ID NO: 20, SEQ ID NO: 59, SEQ ID NO:62, SEQ ID NO:65, SEQ ID NO:68, SEQ ID NO:71, SEQ ID NO:74, SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 26, SEQ ID NO: 128, SEQ ID NO: 131, SEQ ID NO: 134, SEQ ID NO: 137, SEQ ID NO: 140, SEQ ID NO: 143, SEQ ID NO: 29, SEQ ID NO: 146, SEQ ID NO: 149, SEQ ID NO: 152, SEQ ID NO: 155, SEQ ID NO: 32, SEQ ID NO: 158, SEQ ID NO: 161, SEQ ID NO: 164, SEQ ID NO: 167, SEQ ID NO: 170, SEQ ID NO: 173, SEQ ID NO: 35, SEQ ID NO: 38, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200 or SEQ ID NO: 203 or sequences complementary thereto, or sequences comprising at least 90% nucleic acid sequence identity thereof, or a labeled derivatives thereof. A kit can also contain one or more components selected from a group consisting of: at least one enzyme; dNTPs, at least one buffer, at least one salt, at least one control nucleic acid sample and an instruction protocol.

Some embodiments describe kits for detection of a Shiga toxin-producing E. coli (STEC) microorganism comprising: one or more pairs of forward and reverse polymerase chain reaction (PCR) primers selected from a first primer set having SEQ ID NO: 15 and SEQ ID NO 16; a second primer set having SEQ ID NO: 18 and SEQ ID NO 19; a third primer set having SEQ ID NO:21 and SEQ ID NO:22; a fourth primer set having SEQ ID NO: 24 and SEQ ID NO 25, a fifth primer set having SEQ ID NO:27 and SEQ ID NO:28; a sixth primer set having SEQ ID NO: 30 and SEQ ID NO 31, or sequences complementary thereto, or sequences comprising at least 90% nucleic acid sequence identity thereto, fragments having at least 10 contiguous nucleotides thereof, or a labeled derivative thereof; optionally at least one probe selected from SEQ ID NO: 3, SEQ ID NO:6, SEQ ID NO 9 and SEQ ID NO:12, or sequences complementary thereto, or sequences comprising at least 90% nucleic acid sequence identity thereof, fragments having at least 10 contiguous nucleotides thereof, or a labeled derivative thereof; and one or more components selected from a group consisting of: at least one enzyme; dNTPs, at least one buffer, at least one salt, at least one control nucleic acid sample and an instruction protocol. In some embodiments, a kit of the disclosure may also comprise one or more pairs of forward and reverse PCR primers further selected from a seventh primer set having SEQ ID NO: 11 and SEQ ID NO: 12 and SEQ ID NO: 13; an eighth primer set having SEQ ID NO: 1 and SEQ ID NO: 2; a ninth primer set having SEQ ID NO:4 and SEQ ID NO:5; a tenth primer set having SEQ ID NO: 6 and SEQ ID NO: 7; an eleventh primer set having SEQ ID NO:8 and SEQ ID NO:9; and further optionally additionally at least one more probe selected from probes having SEQ ID NO: 14, SEQ ID NO: 3, SEQ ID NO: 10, or sequences complementary thereto, or sequences comprising at least 90% nucleic acid sequence identity thereof, or a labeled derivatives thereof.

In some embodiments, kits of the disclosure further comprise one or more pairs of forward and reverse PCR primers further selected from a twelfth primer set having SEQ ID NO: 33 and SEQ ID NO: 34; a thirteenth primer set having SEQ ID NO: 36 and SEQ ID NO: 37; and optionally at least one more probe further selected from probes having SEQ ID NO: 35, SEQ ID NO: 38, or sequences complementary thereto, or sequences comprising at least 90% nucleic acid sequence identity thereof, or labeled derivatives thereof.

Different primers and probes can be labeled with different labels to allow for detection of different amplified products and/or hybridized products.

In the following detailed description, certain aspects and embodiments will become evident. It should be understood that a given embodiment need not have all aspects and features described herein. It should be understood that these aspects and embodiments are merely exemplary and explanatory and are not restrictive of the invention. It is to be understood that both the foregoing general description and the following detailed description are exemplary and explanatory only and are not restrictive of the invention, as claimed.

The accompanying figures, which are incorporated in and constitute a part of this specification, illustrate several exemplary embodiments of the disclosure and together with the description, serve to explain certain teachings. These and other features of the present teachings are set forth herein.

### DESCRIPTION OF THE DRAWINGS

The skilled artisan will understand that the drawings described below are for illustration purposes only. The drawings are not intended to limit the scope of the present teachings in any way.

FIGS. 1A and 1B illustrate eae inclusion and exclusion testing, according to one embodiment of the disclosure;

FIGS. 2A and 2B illustrates results of a multiplex assay versus a single-plex assay, according to one embodiment; and

FIGS. 3A and 3B show results of STEC confirmation assays according to one embodiment.

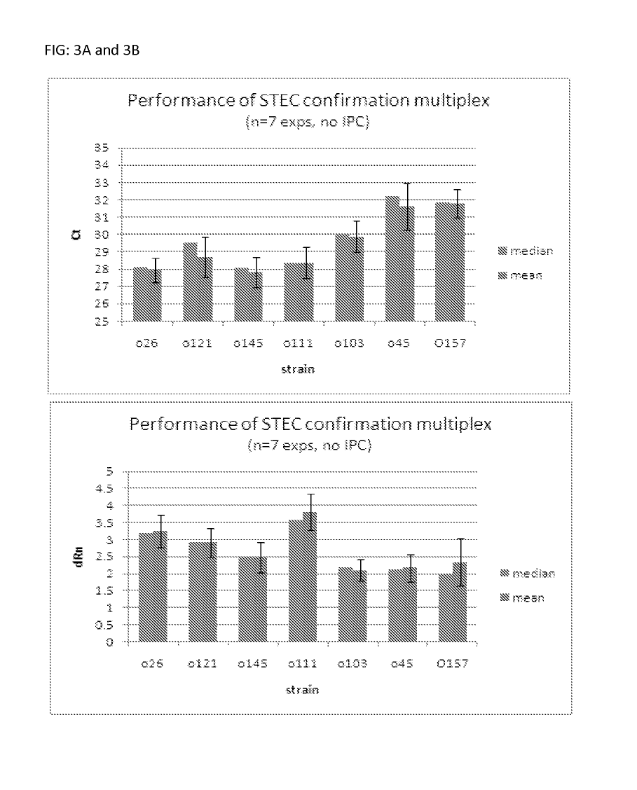

### DETAILED DESCRIPTION

The present disclosure, in some embodiments, describes compositions, kits and methods for detection of one or more STEC microbes including E. coli O157:H7 and non-O157:H7 STEC microorganisms.

Some species of Escherichia coli (E. coli) have the potential to be pathogenic to humans. Among these are the shigatoxigenic group of E. coli (known as STEC or VTEC) which produce shiga toxins (also known as verotoxins). These toxins are proteins encoded by the stx genes (stx1 and/or stx2) and may produce symptoms ranging from simple diarrhea to hemorrhagic diarrhea. Some STEC's may be lethal to humans, especially infants under five years old and immuno-compromised humans. Some STECs not only produce one or more shiga toxins but they can also attach to the intestinal wall because of a protein intimin (an adhesin) encoded by a gene called eae. E. coli carrying these two genes (stx and eae) have previously been identified as being responsible for Uremic and Haemolytic Syndrome (UHS). They are commonly known as Entero-Hemorrhagic Escherichia coli (EHEC). The present specification refers to shiga toxins and eae genes as virulence factors as they contribute to the virulence of STEC microorganisms.

In addition to E. coli O157:H7, the USDA-FSIS has recently identified six strains of non-O157:H7 E. coli STEC including E. coli serotypes O26, O45, O103, O111, O121, O145 as microbes that must be tested as food contaminants to prevent and reduce the incidence of food-borne pathogen outbreaks and fatalities. The European Food Safety Association (EFSA) is also requiring testing of at least four of these E. coli serotypes including O26, O103, O111, O145.

Compositions, kits and methods of the present disclosure provide rapid tests for detecting these microbial contaminants in samples including complex food samples.

Some embodiments relate to compositions comprising isolated nucleic acid sequences that are operable to hybridize to nucleic acid regions that are uniquely found in a STEC microorganism. Nucleic acid regions uniquely found in a STEC microorganism specific are referred to herein as microorganism specific target nucleic acids or STEC microorganism specific target nucleic acids.

Bioinformatic and direct DNA sequence comparisons of several STEC organisms were conducted in an effort to identify various STEC specific target nucleic acid sequences, also referred to herein as microorganism specific target nucleic acids (or nucleic acid regions or target regions). Alignment of these sequences using custom algorithms identified several STEC target nucleic acid regions to which primer pairs described herein were designed for each identified STEC target regions to specifically amplify only the unique STEC target sequences against both inclusion (organism to be detected, i.e., STEC organisms) and exclusion genomes (organisms not to be detected, non-STEC organisms). STEC specific target regions were also used to design probes that can be used to hybridize to and detect STEC specific amplified targets or STEC specific regions in isolated microbial nucleic acids.

In some embodiments, the present disclosure describes isolated nucleic acid sequence comprising SEQ ID NO:15-SEQ ID NO:32, fragments thereof, complements thereof, sequences comprising at least 90% nucleic acid sequence identity thereto and labeled derivatives thereof. These include primer and probe sequences operable to bind to and/or amplify and/or detect and/or identify an E. coli STEC of a strain including O26, O45, O103, O111, O145 and O121.

Primer and probe sequences operable to bind to and/or amplify and/or detect and/or identify an E. coli O157:H7 microorganism are described in SEQ ID NOs: 33-SEQ ID NO: 38 and in SEQ ID NOs: 174-203. Compositions and methods comprising SEQ ID NOs: 33-SEQ ID NO: 38 are also described in U.S. Provisional Patent Application Ser. No. 61/178,931, filed May 15, 2009 (Attorney Docket NO. LT00032Pro); U.S. patent application Ser. No. 12/780,707 filed May 14, 2010 (Attorney Docket NO. LT00032US); PCT Application Serial No. PCT/US2010/034998, filed May 14, 2010 (Attorney Docket NO. LT00032PCT); and European Patent Application Serial NO. 10720105.5, filed May 14, 2010, (Attorney Docket NO. LT00032EP), the entire disclosures of which are incorporated herein by reference.

Some embodiments relate to compositions comprising isolated nucleic acid sequences that are operable to hybridize to nucleic acid regions corresponding to virulence factors expressed in a microorganism. Nucleic acid regions corresponding to virulence factors are referred to herein as virulence factor specific target nucleic acids. Some examples of virulence factors are the stx and eae encoding genes, alleles and/or variants thereof.

In some embodiments, the present disclosure describes isolated nucleic acid sequence comprising SEQ ID NO:11-SEQ ID NO:14, fragments thereof, complements thereof, sequences comprising at least 90% nucleic acid sequence identity thereto and labeled derivatives thereof. These include primer and probe sequences operable to bind to and/or amplify and/or detect and/or identify any of the approximately 25 alleles of the eae gene, encoding intimin.

Virulence factors may, in some embodiments, comprise one or more genes, alleles and/or variants thereof encoding for a shiga toxin. Shiga toxins are a family of related toxins with two major groups, Stx1 and Stx2, whose genes encoded by the loci termed stx1 and stx2 respectively, which are considered to be part of the genome of certain lambdoid prophages. These toxins were first described in explaining the bacterial origin of dysentery caused by Shigella dysenteriae. The most common sources for shiga toxins are the bacteria S. dysenteriae and the shigatoxigenic group of Escherichia coli (STEC), which includes serotypes O157:H7, and other enterohemorrhagic E. coli (EHEC).

Primer and probe sequences operable to bind to and/or amplify and/or detect and/or identify a stx gene are described in SEQ ID NO. 1-SEQ ID NO. 10. Compositions comprising these sequences are also described in U.S. Provisional Patent Application Ser. No. 61/527,107, filed Aug. 24, 2011 (Attorney Docket NO. LT00569Pro) and in U.S. patent application Ser. No. 13/594,682, filed Aug. 24, 2012 (Attorney Docket NO. LT00569), the entire disclosure of which is incorporated herein by reference.

In some embodiments, the present disclosure describes designing degenerate primers. In some embodiments the present disclosure describes designing multiplex primers that may be suitable for multiplex PCR type of assays.

In some embodiments, isolated nucleic acid sequence compositions of the disclosure including primers and probes may further comprise one or more label, such as, but not limited to, a dye, a radioactive isotope, a chemiluminescent label, a fluorescent moiety, a bioluminescent label an enzyme, and combinations thereof.

The present application, in some embodiments describes methods for detecting various STEC strains of E. coli such as an E. coli O157:H7, an E. coli O26, an E. coli O45, an E. coli O103, an E. coli O111, an E. coli O121 and an E. coli O145 in a sample, wherein detection of at least one nucleic acid sequence that is expressed in a STEC organism is indicative of the presence of a STEC organism in the sample. Methods to exclude the presence of STEC E. coli in a sample are also described wherein the absence of detection of any nucleic acid sequence unique to a STEC organism is indicative of the absence of a STEC organism in the sample.

Some amplification methods disclosed here are singleplex methods and some methods disclosed here are multiplex methods. Primers designed herein can be used in both singleplex and multiplex PCR methods.

A method of the disclosure, in some embodiments, can comprise detecting, in a sample, at least one (or more) nucleic acid sequence(s) having at least 10 to at least 25 nucleic acids of one (or more) nucleic acids unique to a STEC organism, or a complementary sequences thereof, wherein detection of at least one nucleic acid sequence unique to a STEC microbe indicates the presence of STEC organism in the sample. Methods of detection may also comprise identification steps. These embodiments are described in detail in sections below of this application.

A methods for detection of a STEC E. coli strain in a sample can comprise: hybridizing a first pair of nucleic acid amplification to a first STEC E. coli strain target polynucleotide sequence present in the sample; amplifying at least a first STEC E. coli strain target polynucleotide sequence or a fragment or a complement thereof to obtain at least one amplified STEC E. coli strain target polynucleotide sequence; and detecting the at least one amplified STEC E. coli strain target polynucleotide sequence; wherein detection of the at least one amplified STEC E. coli strain target polynucleotide sequence is indicative of the presence of the STEC E. coli strain in the sample; and the method optionally further comprising using a probe to detect the at least one amplified the STEC E. coli strain target polynucleotide sequence. More than one primer pairs can be hybridized to detect more than one STEC E. coli strains specific target polynucleotide sequences.

Some embodiments describe methods for detection of an E. coli O121 in a sample comprising: hybridizing at least a first pair of PCR primers comprising nucleic acids of SEQ ID NO: 15 and SEQ ID NO: 16, or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O121 in the sample. A method to detect E. coli O121 may further comprise using a probe to detect the at least one amplified target polynucleotide sequence wherein the probe comprises a sequence of SEQ ID NO: 17, SEQ ID NO: 41, SEQ ID NO:44, SEQ ID NO:47, SEQ ID NO:50, SEQ ID NO:53, or SEQ ID NO:56, fragments having at least 10 contiguous nucleotides thereof, complements thereof, and sequences having at least 90% homology thereto.

Some embodiments describe methods for detection of an E. coli O145 in a sample comprising: hybridizing at least a first pair of PCR primers comprising nucleic acids of SEQ ID NO: 18 and SEQ ID NO: 19, SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O145 in the sample. A method to detect an E. coli O145 may further comprise using a probe to detect the at least one amplified target polynucleotide sequence wherein the probe comprises a sequence of SEQ ID NO: 20, SEQ ID NO: 59, SEQ ID NO:62, SEQ ID NO:65, SEQ ID NO:68, SEQ ID NO:71, or SEQ ID NO:74, fragments having at least 10 contiguous nucleotides thereof, complements thereof, and sequences having at least 90% homology thereto.

Some embodiments describe methods for detection of an E. coli O26 in a sample and comprise: hybridizing at least a first pair of PCR primers comprising nucleic acids of SEQ ID NO: 21 and SEQ ID NO: 22, or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O26 in the sample. A method to detect an E. coli O26 may further comprise using a probe to detect the at least one amplified target polynucleotide sequence wherein the probe comprises a sequence of SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, or SEQ ID NO: 125, fragments having at least 10 contiguous nucleotides thereof, complements thereof, and sequences having at least 90% homology thereto.

Some embodiments describe methods for detection of an E. coli O45 in a sample comprising: hybridizing at least a first pair of PCR primers comprising nucleic acids of SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O45 in the sample. A method to detect an E. coli O45 may further comprise using a probe to detect the at least one amplified target polynucleotide sequence wherein the probe comprises a sequence of SEQ ID NO: 26, SEQ ID NO: 128, SEQ ID NO: 131, SEQ ID NO: 134, SEQ ID NO: 137, SEQ ID NO: 140, or SEQ ID NO: 143, fragments having at least 10 contiguous nucleotides thereof, complements thereof, and sequences having at least 90% homology thereto.

Some embodiments describe methods for detection of an E. coli O103 in a sample comprising: hybridizing at least a first pair of PCR primers comprising nucleic acids of SEQ ID NO: 27 and SEQ ID NO: 28, or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O103 in the sample. A method to detect an E. coli O103 may further comprise using a probe to detect the at least one amplified target polynucleotide sequence wherein the probe comprises a sequence of SEQ ID NO: 29, SEQ ID NO: 146, SEQ ID NO: 149, SEQ ID NO: 152, or SEQ ID NO: 155, fragments having at least 10 contiguous nucleotides thereof, complements thereof, and sequences having at least 90% homology thereto.

Some embodiments describe methods for detection of an E. coli O111 in a sample comprising: hybridizing at least a first pair of PCR primers comprising nucleic acids of SEQ ID NO: 30 and SEQ ID NO: 31, or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O111 in the sample. A method to detect an E. coli O111 can further comprise using a probe to detect the at least one amplified target polynucleotide sequence wherein the probe comprises a sequence of SEQ ID NO: 32, SEQ ID NO: 158, SEQ ID NO: 161, SEQ ID NO: 164, SEQ ID NO: 167, SEQ ID NO: 170, or SEQ ID NO: 173, fragments having at least 10 contiguous nucleotides thereof, complements thereof, and sequences having at least 90% homology thereto.

In some example embodiments, a method for detection of an E. coli O157:H7 in a sample comprises: hybridizing at least a first pair of PCR primers comprising nucleic acids of SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202 complements thereof, labeled derivatives thereof, and sequences having at least 90% homology thereto to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O015:H7 in the sample. A method to detect an E. coli O157:H7 can further comprise using a probe to detect the at least one amplified target polynucleotide sequence wherein the probe comprises a sequence of SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200, or SEQ ID NO: 203, complements thereof, labeled derivatives thereof, and sequences having at least 90% homology thereto.

Detection of STEC strains described here can use a polymerase chain reaction (PCR) for rapid amplification and detection of target regions. In general all methods of the disclosure include comparing the detection or absence of an STEC organism using a suitable control, for example, an internal positive control may be used in a PCR reaction which will have a detectable signal/positive result, and a suitable negative control may also be used that will have no detectable signal.

In some embodiments, methods of detecting in a sample the presence of STEC microorganisms can further comprise detecting the presence of a stx encoding nucleic acid and/or an eae encoding nucleic acid.

The specification, in some embodiments, describes a method for detection of one or more microorganisms, each microorganism expressing one or more virulence factors comprising: 1) detecting the presence of one or more virulence factors comprising: a) contacting a sample suspected of containing one or more microorganisms expressing one or more virulence factors with one or more sets of primers specific to the one or more virulence factors; b) amplifying at least one target nucleic acid sequence encoding at least one of the virulence factors (in some examples by a multiplex amplification method if more than one virulence factors are to be co-detected if present) to obtain one or more amplified virulence factor specific nucleic acids; c) detecting the one or more amplified virulence factor specific nucleic acids or fragments or complements thereof; and d) optionally identifying the amplified virulence factor specific nucleic acids, wherein detecting the one or more amplified virulence factor specific nucleic acids or fragments or complements thereof is indicative of the presence of a microorganism in the sample expressing the one or more virulence factor; and 2) determining the strain of the one or more microorganism expressing the one or more virulence factor comprising: a) contacting the sample with at least one set of primers specific to the strain of the one or more microorganism; b) amplifying (or co-amplifying in a multiplex amplification method along with virulence factor detection) at least one strain specific target nucleic acid sequence encoding for nucleic acid targets specific to at least one strain of the microorganism to obtain one or more amplified strain specific nucleic acids; c) detecting the one or more amplified strain specific nucleic acid or fragments or complements thereof; and d) optionally identifying the amplified strain specific nucleic acids. In some embodiments of the method each set of primers specific to the one or more virulence factors is labeled with a different label. In some embodiments of the method, each set of primers specific to the one or more strain of the microorganism is labeled with a different label and wherein each set of primers specific to the one or more virulence factors is labeled with a different label.

In some embodiments, steps described in the method above of 1) detecting presence of one or more virulence factors and 2) determining strain of the one or more microorganism expressing the one or more virulence factor can be performed simultaneously or consequently and/or in any order.

In some embodiments of a method for detection of one or more microorganisms expressing one or more virulence factors, the one or more sets of oligonucleotide primers specific to the one or more virulence factors each comprise: at least one forward primer and at least a reverse primer operable to hybridize to one or more virulence factors. Virulence factors in some embodiments comprise but are not limited to one or more of the following: all variants of shiga toxin (stx) encoding nucleic acids, alleles and fragments thereof; and/or all variants of eae gene encoding nucleic acids, alleles and fragments thereof. A stx encoding nucleic acid can comprise an stx1 gene, a stx2 gene, an allele of an stx1 gene an allele of an stx2 gene, or a fragments thereof. Table 1 below lists several stx1 and stx2 subtypes and variants that are detected by the methods described herein.

TABLE 1

| Stx Type | Stx Subtypes | Stx variants |
|---|---|---|
| Stx1 | 3 | 1a, 1c, 1d |
| Stx2 | 7 | 2a, 2b, 2c, 2d, 2e, 2f, 2g |

A method of detecting all stx variants, including stx1 (1a, 1c and 1d) and stx2 (2a, 2b, 2c, 2d, 2e, 2f and 2g) comprise detecting all know variants of stx1 and stx2 and comprise performing a multiplex assay comprising hybridizing at least two pairs of PCR primers selected from PCR primer pairs having nucleic acid sequences of SEQ ID NO: 1 and SEQ ID NO: 2; and/or SEQ ID NO: 4 and SEQ ID NO: 5; and/or SEQ ID NO: 6 and SEQ ID NO: 7 and/or SEQ ID NO: 8 and SEQ ID NO: 9, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an stx encoding nucleic acid in the sample. In some embodiments, all the four primer pairs described above are used to amplify one or more target stx nucleic acid sequences in the sample and wherein the detection of at least one amplified target stx nucleic acid is indicative of the presence of a microorganism having a stx encoding loci in its nucleic acids. A method for detecting a microbe expressing an stx gene may further comprise using a probe to detect the at least one amplified target polynucleotide sequence, wherein the probe comprises a sequence of SEQ ID NO: 3 (to detect an stx nucleic acid product amplified by using primer pair having SEQ ID NO. 1 and SEQ ID NO. 2), wherein the probe comprises a sequence of SEQ ID NO: 10 (to detect an stx nucleic acid product amplified by using primer pair having SEQ ID NO. 4 and SEQ ID NO. 5; and/or SEQ ID NO. 6 and SEQ ID NO. 7 and SEQ ID NO. 8 and SEQ ID NO. 9. Probes having SEQ ID NO: 3 and SEQ ID NO: 10 may also comprise fragments having at least 10 contiguous nucleotides thereof, complements thereof, and sequences having at least 90% homology thereto. All stx variants may be detected using these methods due to degenerate primers and probes that contain miss-matched bases at known polymorphic sites. Methods for detecting stx expressing microbes and compositions comprising the primer-probe sequences are also described in U.S. Provisional Patent Application Ser. No. 61/527,107, filed Aug. 24, 2011 (Attorney Docket NO. LT00569Pro), the entire disclosure of which is incorporated herein by reference.

An eae encoding nucleic acid may comprise an eae gene, an allele of an eae gene, or a fragments thereof. Microorganisms expressing one or more virulence factors include for example Shiga toxin-producing E. coli (STEC).

A method of detecting the presence of an eae expressing microbe in a sample comprises hybridizing at least one pair of PCR primers such as a primer having nucleic acid sequences of SEQ ID NO: 11 and SEQ ID NO:12 and/or SEQ ID NO: 13, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an eae encoding nucleic acid in the sample. A method for detecting a microbe expressing an eae gene may further comprise using a probe to detect the at least one amplified target polynucleotide sequence, wherein the probe comprises a sequence of SEQ ID NO: 14 (to detect an eae nucleic acid product amplified by using primer pairs having SEQ ID NO. 11 and SEQ ID NO. 12 or SEQ ID NO. 13), fragments thereof and complements thereof and sequences having at least 90% sequence homology thereto.

In some embodiments of a method described above the step 1) of determining the presence of one or more virulence factor, the one or more primer sets specific for one or more virulence factors may comprise: a first primer set having SEQ ID NO: 1 and SEQ ID NO 2; a second primer set having SEQ ID NO: 4 and SEQ ID NO 5; a third primer set having SEQ ID NO: 6 and SEQ ID NO:7; a fourth primer set having SEQ ID NO: 8 and SEQ ID NO: 9; and/or a fifth primer set having SEQ ID NO: 11, SEQ ID NO: 12; and SEQ ID NO: 13. A method may further comprise the steps of detecting the one or more amplified virulence factor specific nucleic acids or fragments or complements thereof comprises hybridizing the one or more amplified virulence factor specific nucleic acids or fragments or complements thereof with a probe selected from SEQ ID NO: 3, SEQ ID NO: 10, and SEQ ID NO: 14.

In other embodiments, not detecting any amplified product using the methods described above may be used to exclude the presence of a microorganism expressing a virulence factor in a sample.

In some embodiments of a method as described above, the step 2) of determining the strain of the one or more microorganism expressing the one or more virulence factor, the at least one set of oligonucleotide primers specific to the strain of the one or more microorganism may comprise a first primer set comprises one or more primers selected from SEQ ID NO: 15 and SEQ ID NO 16; or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55; a second primer set comprises one or more primers selected from SEQ ID NO: 18 and SEQ ID NO 19; SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74; a third primer set comprises one or more primers selected from SEQ ID NO: 21 and SEQ ID NO: 22; or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124; a fourth primer set comprises one or more primers selected from SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142; a fifth primer set comprises one or more primers selected from SEQ ID NO: 27, SEQ ID NO: 28; or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154; a sixth primer set comprises one or more primers selected from SEQ ID NO: 30 and SEQ ID NO: 31; or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172; a seventh primer set comprises one or more primers selected from SEQ ID NO: 33 and SEQ ID NO 34 or SEQ ID NO: 36 and SEQ ID NO: 37, or SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202. In some embodiments, the steps of detecting the one or more amplified strain specific nucleic acids or fragments or complements thereof comprises hybridizing the one or more amplified strain specific nucleic acids or fragments or complements thereof with a probe selected from SEQ ID NO: 17, SEQ ID NO: 41, SEQ ID NO:44, SEQ ID NO:47, SEQ ID NO:50, SEQ ID NO:53, SEQ ID NO:56, SEQ ID NO: 20, SEQ ID NO: 59, SEQ ID NO:62, SEQ ID NO:65, SEQ ID NO:68, SEQ ID NO:71, SEQ ID NO:74, SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 26, SEQ ID NO: 128, SEQ ID NO: 131, SEQ ID NO: 134, SEQ ID NO: 137, SEQ ID NO: 140, SEQ ID NO: 143, SEQ ID NO: 29, SEQ ID NO: 146, SEQ ID NO: 149, SEQ ID NO: 152, SEQ ID NO: 155, SEQ ID NO: 32, SEQ ID NO: 158, SEQ ID NO: 161, SEQ ID NO: 164, SEQ ID NO: 167, SEQ ID NO: 170, SEQ ID NO: 173, SEQ ID NO: 35, SEQ ID NO: 38, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200 or SEQ ID NO: 203.

In some embodiments, detecting an amplified nucleic acid comprises one or more methods such as but not limited to hybridization, mass spectrometry, molecular barcoding, microfluidics, chemiluminescence, enzyme technologies and combinations thereof. Some embodiments may also comprise identifying a microbe and may comprise methods such as DNA sequencing.

In some embodiments of the present methods, one assay alone may not be definitive for detecting a STEC organism due to genomic similarity between the genomic regions of other non-STEC organisms. Yet, when two (or more) assays such as but not limited to the assays shown in Table 2 are used either in parallel or as a multiplex assay, e.g., in a real-time TaqMan® assay, for example, where each probe in each of the two (or more) assays has a different label for distinguishing results on a real-time PCR instrument, e.g., a 7500 Fast Real-Time PCR System (Applied Biosystems), a positive result from such an assay is indicative of the presence of an STEC organism.

In other embodiments, dual or multiplex (more than 2 assay sets) assay approach can be used to detect and distinguish STEC organism strains from each other as well as detecting and distinguishing expression of stx alleles/variants and/or eae. In some embodiments, assay may comprise detecting the presence of genes, alleles and/or fragments of such genes/alleles encoding one or more shiga-toxin encoding gene loci such as but not limited to stx1 and stx2. Some embodiments describe detecting all these gene loci as positive identification of a shiga toxin producing organism. Some embodiments describe detecting at least two of these gene loci as positive identification of a shiga toxin producing organism. Some embodiments describe detection of an eae loci. Some embodiments describe detection of different amplification products for different STEC strains such as an E. coli O157:H7, an E. coli O26, an E. coli O45, an E. coli O103, an E. coli O111, an E. coli O121 and an E. coli O145.

Methods may include multiplex assays such as polymerase chain reactions, wherein hybridizing and amplifying of said first pair of polynucleotide primers occurs in a first vessel and said hybridizing and amplifying of said second pair of polynucleotide primers occurs in a second vessel, or hybridizing and amplifying of said first pair of polynucleotide primers and said hybridizing and amplifying of said second pair of polynucleotide primers occurs in a single vessel, the detection is a real-time assay, the real-time assay is a SYBR® Green dye assay or a TaqMan® assay. Methods may also comprise using additional primers such as a third primer pair and a fourth primer pair and so on.

A method of the disclosure may further comprise providing a first probe and a second probe (and additional probes such as a third probe and a fourth probe and so on), wherein the first and second probes are different from each other, the first probe operable to identify the first amplified target polynucleotide sequence and the second probe operable to identify the second amplified target nucleotide sequence, the first probe further comprises a first label and said second probe further comprises a second label, wherein both labels are selected from a dye, a radioactive isotope, a chemiluminescent label, and an enzyme, the dye comprises a fluorescein dye, a rhodamine dye, or a cyanine dye, the dye is a fluorescein dye and first probe is labeled with FAM™ dye and said second probe is labeled with VIC® dye; and hybridizing the first and second probes to the PCR amplified fragments to detect the presence of the first amplified target polynucleotide sequence and the second amplified target polynucleotide sequence from the sample.

In some embodiments, the disclosure describes workflows of methods for detecting and identifying one or more Shiga toxin-producing E. coli (STEC) microorganisms in a sample comprising the steps of: a) immunomagnetic separation of STEC microorganisms present in the sample from other sample components and other microbes in the sample to obtain separated STEC microorganisms; b) optionally enriching the separated STEC microorganisms; c) performing a multiplex polymerase chain reaction (PCR) on the separated STEC microorganisms to amplify and detect the presence of at least one nucleic acids selected from: nucleic acids encoding for a shiga toxin gene, including nucleic acids encoding stx1 and stx2; nucleic acids encoding an eae gene; nucleic acids specific for an E. coli strain O157:H7, wherein the detection of at least one nucleic acid above indicates the presence of an STEC microorganism or E. coli O157:H7 or both; and d) if a nucleic acid is detected in step c) performing a second round of multiplex PCR with primers specific to amplify and detect the presence one or more nucleic acids encoding for target regions associated with one or more STEC microorganisms including an E. coli O157:H7, an E. coli O26, an E. coli O45, an E. coli O103, an E. coli O111, an E. coli O121 and an E. coli O145.

In some embodiments, the disclosure describes workflows of methods for detecting and identifying one or more Shiga toxin-producing E. coli (STEC) microorganisms in a sample comprising the steps of: a) separation of STEC microorganisms present in the sample from other sample components and other microbes in the sample to obtain separated STEC microorganisms; b) optionally enriching the STEC microorganisms (separated or un-separated from the sample); c) performing a multiplex polymerase chain reaction (PCR) on the separated STEC microorganisms to amplify and detect the presence of at least one nucleic acids selected from: nucleic acids encoding for a shiga toxin gene, including nucleic acids encoding stx1 and stx2; nucleic acids encoding an eae gene; nucleic acids specific for an E. coli strain O157:H7, wherein the detection of at least one nucleic acid above indicates the presence of an STEC microorganism or E. coli O157:H7 or both; and d) if a nucleic acid is detected in step c) performing a second round of multiplex PCR with primers specific to amplify and detect the presence one or more nucleic acids encoding for target regions associated with one or more STEC microorganisms including an E. coli O157:H7, an E. coli O26, an E. coli O45, an E. coli O103, an E. coli O111, an E. coli O121 and an E. coli O145.

In some embodiments, an exemplary method of detecting the presence of a STEC organisms in a sample can also comprise 1) optionally separating or isolating a STEC microorganism present in a sample from other sample components; and 2) extracting or isolating nucleic acid from a sample; 3) detecting the presence of at least one unique nucleic acid target region of STEC serotypes O157:H7, O26, O45, O103, O111, O121, and O145 and/or fragments thereof and/or complement thereof; and 4) detecting the presence of STEC virulence factors such as stx and/or eae and/or fragments thereof and/or complement thereof.

In other embodiments, not detecting any amplified product using the methods described above may be used to exclude the presence of a STEC microorganism in a sample.

Amplification of target nucleic acids of targets including stx1, stx2, eae, E. coli O157:H7, an E. coli O26, an E. coli O45, an E. coli O103, an E. coli O111, an E. coli O121 and an E. coli O145 can be detected by using a plurality of labels on each different set of primers and/or probes. For example, in step c) above primers and/or probes with a different label for each of stx1, sxt2, eae and E. coli O157:H7 may be used and in step d) primers and/or a probes with a different label each may be used for the various microorganisms.

Magnetic beads used in immunomagnetic separation have one or more antibodies on them comprising antibodies operable to bind to an E. coli O157:H7, an E. coli O26, an E. coli O45, an E. coli O103, an E. coli O111, an E. coli O121, and an E. coli O145. Non-limiting examples of magnetic beads include Dynabeads (Life Technologies AS, Invitrogen Dynal AS).

In some embodiments, methods of the disclosure can be performed on an automated system. Automation decreases the time as well as efficiency and allows processing multiples samples. Automated systems may comprise a magnetic separation platform such as but not limited to MagMAX™ Express-96 Magnetic Particle Processor Platform (Life Technologies Corporation), Pathatrix (Matrix Microsciences).

In a non-limiting example, immunomagnetic beads were used to capture bacteria from complex food sample matrices and detection of 1-3 cfu (colony forming units) of E. coli O157:H7 was shown from a sample comprising 375 g of ground beef and beef trim in less than 8 hours of time-to-result. The method in this example was fully automated on a MagMax™ Express-96 platform and enables a rapid, simple and user-friendly workflow.

Immunomagnetic capturing of bacterial pathogens as described here is followed by lysis of captured bacteria directly in PCR reactions. The present method circumvents the need for nucleic acid extraction (RNA/DNA) prior to the PCR since the immunomagnetic beads are PCR compatible and bead-bacterial complexes can be added directly to PCR reactions. Automation on a magnetic separation platform and PCR processing platform increases speed and reduces user time.

Compositions and methods of the present disclosure are ideally suited for the preparation of a kit suitable for detecting the presence of a STEC organism. Such a kit may comprise at least one set of oligonucleotide primers for simultaneous use in a multiplex PCR process for the detection of a virulence factors associated with STEC microbes, said set of primers comprising a primers set operable to hybridize to (are directed towards) virulence factors comprising all variants of shiga toxin and eae. In some embodiments, a kit may further or alternatively comprise one or more primer sets operable to hybridize to unique nucleic acid targets of one or more strains of STEC such as but not limited to an E. coli O157:H7, an E. coli O26, an E. coli O45, an E. coli O103, an E. coli O111, an E. coli O121 and an E. coli O145. Kits may additionally comprise one or more reagents such as but are not limited to, buffers, nucleotide triphosphates, DNA polymerases, intercalating dye, primers, probes, salt, and instructions for the use of the kit.

An example kit for detection of a Shiga toxin-producing E. coli (STEC) microorganism can comprise: one or more pairs of forward and reverse polymerase chain reaction (PCR) primers selected from a first primer set comprising one or more primers selected from SEQ ID NO: 15 and SEQ ID NO 16, or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55; a second primer set comprising one or more primers selected from SEQ ID NO: 18 and SEQ ID NO 19, SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74; a third primer set comprising one or more primers selected from SEQ ID NO:21 and SEQ ID NO:22, or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124; a fourth primer set comprising one or more primers selected from SEQ ID NO: 24 and SEQ ID NO 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142; a fifth primer set comprising one or more primers selected from SEQ ID NO:27 and SEQ ID NO:28, or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154; a sixth primer set comprising one or more primers selected from SEQ ID NO: 30 and SEQ ID NO: 31; or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172; and a seventh primer set comprising one or more primers selected from SEQ ID NO: 33 and SEQ ID NO 34 or SEQ ID NO: 36 and SEQ ID NO: 37, or SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202, or sequences complementary to any of these primers, or sequences comprising at least 90% nucleic acid sequence identity thereto, fragments having at least 10 contiguous nucleotides thereof, or a labeled derivative thereof. In some embodiments, a kit of the disclosure can further comprise one or more pairs of forward and reverse PCR primers further selected from an eighth primer set having SEQ ID NO: 11 and SEQ ID NO: 12 and SEQ ID NO: 13; a ninth primer set having SEQ ID NO: 1 and SEQ ID NO: 2; a tenth primer set having SEQ ID NO:4 and SEQ ID NO:5; an eleventh primer set having SEQ ID NO: 6 and SEQ ID NO: 7; a twelfth primer set having SEQ ID NO:8 and SEQ ID NO:9. A kit can further comprise one more probe selected from probes having SEQ ID NO: 3, SEQ ID NO: 10, SEQ ID NO: 14, SEQ ID NO: 17, SEQ ID NO: 41, SEQ ID NO:44, SEQ ID NO:47, SEQ ID NO:50, SEQ ID NO:53, SEQ ID NO:56, SEQ ID NO: 20, SEQ ID NO: 59, SEQ ID NO:62, SEQ ID NO:65, SEQ ID NO:68, SEQ ID NO:71, SEQ ID NO:74, SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 26, SEQ ID NO: 128, SEQ ID NO: 131, SEQ ID NO: 134, SEQ ID NO: 137, SEQ ID NO: 140, SEQ ID NO: 143, SEQ ID NO: 29, SEQ ID NO: 146, SEQ ID NO: 149, SEQ ID NO: 152, SEQ ID NO: 155, SEQ ID NO: 32, SEQ ID NO: 158, SEQ ID NO: 161, SEQ ID NO: 164, SEQ ID NO: 167, SEQ ID NO: 170, SEQ ID NO: 173, SEQ ID NO: 35, SEQ ID NO: 38, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200 or SEQ ID NO: 203 or sequences complementary thereto, or sequences comprising at least 90% nucleic acid sequence identity thereof, or a labeled derivatives thereof. A kit can also contain one or more components selected from a group consisting of: at least one enzyme; dNTPs, at least one buffer, at least one salt, at least one control nucleic acid sample and an instruction protocol.

Another exemplary kit for detection of a Shiga toxin-producing E. coli (STEC) microorganism comprises: one or more pairs of forward and reverse polymerase chain reaction (PCR) primers selected from a first primer set having SEQ ID NO: 15 and SEQ ID NO 16; a second primer set having SEQ ID NO: 18 and SEQ ID NO 19; a third primer set having SEQ ID NO:21 and SEQ ID NO:22; a fourth primer set having SEQ ID NO: 24 and SEQ ID NO 25, a fifth primer set having SEQ ID NO:27 and SEQ ID NO:28; a sixth primer set having SEQ ID NO: 30 and SEQ ID NO 31, or sequences complementary thereto, or sequences comprising at least 90% nucleic acid sequence identity thereto, fragments having at least 10 contiguous nucleotides thereof, or a labeled derivative thereof; optionally at least one probe and one or more components selected from a group consisting of: at least one enzyme; dNTPs, at least one buffer, at least one salt, at least one control nucleic acid sample and an instruction protocol. Such as kit can detect the presence of one or more STEC organisms including an E. coli O26, an E. coli O45, an E. coli O103, an E. coli O111, an E. coli O121 and an E. coli O145.

In some embodiments, a kit of the disclosure may also or alternatively comprise one or more pairs of forward and reverse PCR primers further selected from a seventh primer set having SEQ ID NO: 11 and SEQ ID NO: 12 and SEQ ID NO: 13; an eighth primer set having SEQ ID NO: 1 and SEQ ID NO: 2; a ninth primer set having SEQ ID NO:4 and SEQ ID NO:5; a tenth primer set having SEQ ID NO: 6 and SEQ ID NO: 7; an eleventh primer set having SEQ ID NO:8 and SEQ ID NO:9; and further optionally additionally at least one more probe selected from probes having SEQ ID NO: 14, SEQ ID NO: 3, SEQ ID NO: 10, or sequences complementary thereto, or sequences comprising at least 90% nucleic acid sequence identity thereof, or a labeled derivatives thereof. Such as kit can detect the presence of one or more STEC organisms expressing virulence factors stx and eae. If these primer/probe combinations are in addition to the primer probes described in the kits described in the paragraph directly above, these kits can also detect the presence of an E. coli O26, an E. coli O45, an E. coli O103, an E. coli O111, an E. coli O121 and an E. coli O145.

In some embodiments, kits of the disclosure can further comprise additionally one or more pairs of forward and reverse PCR primers further selected from a twelfth primer set having SEQ ID NO: 33 and SEQ ID NO: 34; a thirteenth primer set having SEQ ID NO: 36 and SEQ ID NO: 37; and optionally additionally at least one more probe further selected from probes having SEQ ID NO: 35, SEQ ID NO: 38, or sequences complementary thereto, or sequences comprising at least 90% nucleic acid sequence identity thereof, or labeled derivatives thereof. Such as kit can detect the presence of one or more STEC organisms including an E. coli O157:H7.

In some embodiments, kit primers may be labeled. A kit comprising multiple pairs of primers may have primer pairs each labeled with different labels that may be detectable separately. Probes comprised in kits of the disclosure may be labeled. If a kit comprises multiple probes each probe may be labeled with a different label to allow detection of different products that may be the target of each different probe. Components may be provided as solutions or as lyophilized powders which may be later reconstituted if needed in solutions and/or buffers which may also be provided.

Components of kits may be individually and in various combinations comprised in one or a plurality of suitable container means. It is within the scope of these teachings to provide test kits for use in manual applications or test kits for use with automated sample preparation, reaction set-up, detectors or analyzers. In some embodiments, a kit amplification product may be further analyzed by methods such as but not limited to electrophoresis, hybridization, mass spectrometry, molecular barcoding, microfluidics, chemiluminescence and/or enzyme technologies.

For the purposes of interpreting of this specification, the following definitions may apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of and/or “consisting of:”

As used herein, the phrase “nucleic acid,” “nucleic acid sequence,” “oligonucleotide”, and polynucleotide(s)” are interchangeable and not intended to be limiting.

As used herein, the phrase “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “specific hybridization” refers to hybridization between two polynucleotides under stringent hybridization conditions.

As used herein, the term “polynucleotide” refers to a polymeric form of nucleotides of any length, either ribonucleotides, deoxynucleotides, or peptide nucleic acids (PNA), and includes both double- and single-stranded RNA, DNA, and PNA. A polynucleotide may include nucleotide sequences having different functions, including, for instance, coding regions, and non-coding regions such as regulatory regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “oligonucleotide” refers to a polynucleotide of the present invention, typically a primer and/or a probe.

As used herein a “target-specific polynucleotide” refers to a polynucleotide having a target-binding segment that is perfectly or substantially complementary to a target sequence, such that the polynucleotide binds specifically to an intended target without significant binding to non-target sequences under sufficiently stringent hybridization conditions. The target-specific polynucleotide can be e.g., a primer or probe and the subject of hybridization with its complementary target sequence.

The term “target sequence”, “target signature sequence” “target nucleic acid”, “target” or “target polynucleotide sequence” refers to a nucleic acid of interest. The target sequence can be a polynucleotide sequence that is the subject of hybridization with a complementary polynucleotide, e.g. a primer or probe. The target sequence can be composed of DNA, RNA, an analog thereof, and including combinations thereof. The target sequence may be known or not known, in terms of its actual sequence and its amplification can be desired. The target sequence may or may not be of biological significance. Typically, though not always, it is the significance of the target sequence which is being studied in a particular experiment. As non-limiting examples, target sequences may include regions of genomic DNA, regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

As used herein an “amplified target polynucleotide sequence product” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” or “amplified product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labeling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2.sup.nd Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labelling of PCR probes and primers” (1995) in PCR 2: A Practical Approach, Oxford University Press, Oxford, pp. 39-54).

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “end-point analysis” refers to a method where data collection occurs only when a reaction is substantially complete.

The term “real-time analysis” refers to periodic monitoring during PCR. Certain systems such as the ABI 7700 Sequence Detection System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

The term “quenching” refers to a decrease in fluorescence of a first moiety (reporter dye) caused by a second moiety (quencher) regardless of the mechanism.

A “primer,” as used herein, is an oligonucleotide that is complementary to a portion of target polynucleotide and, after hybridization to the target polynucleotide, may serve as a starting-point for an amplification reaction and the synthesis of an amplification product. Primers include, but are not limited to, spanning primers . A “primer pair” refers to two primers that can be used together for an amplification reaction. A “PCR primer” refers to a primer in a set of at least two primers that are capable of exponentially amplifying a target nucleic acid sequence in the polymerase chain reaction.

The term “probe” comprises a polynucleotide that comprises a specific portion designed to hybridize in a sequence-specific manner with a complementary region of a specific nucleic acid sequence, e.g., a target nucleic acid sequence. In certain embodiments, the specific portion of the probe may be specific for a particular sequence, or alternatively, may be degenerate, e.g., specific for a set of sequences. In certain embodiments, the probe is labeled. The probe can be an oligonucleotide that is complementary to at least a portion of an amplification product formed using two primers.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary.

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a fluorophore (a fluorescent quencher). This phenomenon is generally known as fluorescent resonance energy transfer (FRET). Alternatively, a quencher can dissipate the energy absorbed from a fluorophore as heat (a non-fluorescent quencher).

As used herein the term “sample” refers to a starting material suspected of harboring a particular microorganism or group of microorganisms. A “contaminated sample” refers to a sample harboring a pathogenic microbe thereby comprising nucleic acid material from the pathogenic microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), water samples, environmental samples (e.g., soil samples, dirt samples, garbage samples, sewage samples, industrial effluent samples, air samples, or water samples from a variety of water bodies such as lakes, rivers, ponds etc.,), air samples (from the environment or from a room or a building), forensic samples, agricultural samples, pharmaceutical samples, biopharmaceutical samples, samples from food processing and manufacturing surfaces, and/or biological samples.

Disclosed are compositions, assays, methods and kits for the specific detection of STEC organisms from samples including clinical samples, food samples, complex food matrices, water, a beverage sample, a fermentation broth, a forensic sample, an environmental sample (e.g., soil, dirt, garbage, sewage, air, or water), including food processing and manufacturing surfaces, or a biological sample.

A sample may be tested directly, or may be prepared or processed in some manner prior to testing. For example, a sample may be processed to enrich any contaminating microbe and may be further processed to separate and/or lyse microbial cells contained therein. Lysed microbial cells from a sample may be additionally processed or prepares to separate, isolate and/or extract genetic material from the microbe for analysis to detect and/or identify the contaminating microbe. In some specific embodiments described here, as sample may be subject to separation to initially separate microbes of interest from other microbes and other sample components. For example, for complex food samples with complex components and immunomagnetic separation methods are described to separate STEC organisms based on using antibodies attached to beads where the antibodies are specific to isolate one or more STEC microbes desired to be detected. Separated samples may be enriched prior to analysis. Analysis of a sample may include one or more molecular methods. For example, according to some exemplary embodiments of the present disclosure, a sample may be subject to nucleic acid amplification (for example by PCR) using appropriate oligonucleotide primers that are specific to one or more microbe nucleic acid sequences that the sample is suspected of being contaminated with. Amplification products may then be further subject to testing with specific probes (or reporter probes) to allow detection of microbial nucleic acid sequences that have been amplified from the sample. In some embodiments, if a microbial nucleic acid sequence is amplified from a sample, further analysis may be performed on the amplification product to further identify, quantify and analyze the detected microbe (determine parameters such as but not limited to the microbial strain, pathogenecity, quantity etc.).

As used herein “preparing” or “preparing a sample” or “processing” or processing a sample” refers to one or more of the following steps to achieve separation of microbes from sample components and in some embodiments optionally extraction and separation of a nucleic acid from a sample: (1) separation of bacterial cells from the sample using for example, immunomagnetic separation, (2) bacterial enrichment, (3) cell lysis, and/or (4) optionally nucleic acid extraction and/or purification (e.g., DNA extraction, total DNA extraction, genomic DNA extraction, RNA extraction). Types of nucleic acid extracted include, but are not limited to, DNA, RNA, mRNA and miRNA.

As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

As used herein, “detecting” or “detection” refers to the disclosure or revelation of the presence or absence in a sample of a target polynucleotide sequence or amplified target polynucleotide sequence product. The detecting can be by end point, real-time, enzymatic, and by resolving the amplification product on a gel and determining whether the expected amplification product is present, or other methods known to one of skill in the art.

The presence or absence of an amplified product can be determined or its amount measured. Detecting an amplified product can be conducted by standard methods well known in the art and used routinely. The detecting may occur, for instance, after multiple amplification cycles have been run (typically referred to an end-point analysis), or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. For example, a convenient label is typically a label that is fluorescent, which may be used in various formats including, but are not limited to, the use of donor fluorophore labels, acceptor fluorophore labels, fluorophores, quenchers, and combinations thereof. Assays using these various formats may include the use of one or more primers that are labeled (for instance, scorpions primers, amplifluor primers), one or more probes that are labeled (for instance, adjacent probes, TaqMan® probes, light-up probes, molecular beacons), or a combination thereof. The skilled person in view of the present teachings will understand that in addition to these known formats, new types of formats are routinely disclosed. The present invention is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction. As an alternative to detection using a labeled primer and/or probe, an amplification product can be detected using a polynucleotide binding dye such as a fluorescent DNA binding dye. Examples include, for instance, SYBR® Green dye or SYBR® Gold dye (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

As used herein, a “target specific polynucleotide” refers to a nucleic acid sequence that is able to specifically hybridize to a gene and/or an allele and/or a portion thereof and/or a complement thereof that encodes a target (shiga toxin, eae, or a target specific to a big 6 pathogen or to E. coli O157:H7), under suitable hybridization conditions and which does not hybridize with other nucleic acid sequences that do not encode for the target or portions thereof or complements thereof. In some embodiments, a “target-specific polynucleotide” of the disclosure is a probe or primer sequence described in SEQ ID NOS: 1-203. It is well within the ability of one skilled in the art, using the present teachings, to determine suitable hybridization conditions based on probe length, G+C content, and the degree of stringency required for a particular application.

It is expected that minor sequence variations in virulence factor specific target nucleotide sequences and microorganism specific target nucleotide sequences associated with nucleotide additions, deletions and mutations, whether naturally occurring or introduced in vitro, would not interfere with the usefulness of the various primer and probe nucleic acid sequences disclosed herein, as would be understood by one of skill in the art. Therefore, the scope of the present invention as claimed is intended to encompass minor variations in the sequences of described here and sequences having at least 90% homology to the primer and probe sequences disclosed herein.

A probe may be RNA or DNA. Depending on the detection means employed, the probe may be unlabeled, radiolabeled, chemiluminescent labeled, enzyme labeled, or labeled with a dye. The probe may be hybridized with a sample in solution or immobilized on a solid support such as nitrocellulose, a microarray or a nylon membrane, or the probe may be immobilized on a solid support, such as a silicon chip or a microarray.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, may depend upon, for example, the nature of the nucleotide sequence, temperature, and buffer conditions. These conditions may also depend on what event is desired, such as hybridization, cleavage, or strand extension. An “isolated” polynucleotide refers to a polynucleotide that has been removed from its natural environment. A “purified” polynucleotide is one that is at least about 60% free, preferably at least about 75% free, and most preferably at least about 90% free from other components with which they are naturally associated.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims. Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). The term “and/or” means one or all of the listed elements or a combination of any two or more of the listed elements.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Q.beta. replicase system (Lizardi et al., 1988, BioTechnology 6:1197 1202), and the techniques disclosed in WO 90/10064 and WO 91/03573.

Examples of techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784).

Other exemplary techniques include Nucleic Acid Sequence-Based Amplification (“NASBA”; see U.S. Pat. No. 5,130,238), Q.beta. replicase system (see Lizardi et al., BioTechnology 6:1197 (1988)), and Rolling Circle Amplification (see Lizardi et al., Nat Genet. 19:225 232 (1998)). The amplification primers of the present invention may be used to carry out, for example, but not limited to, PCR, SDA or tSDA. Any of the amplification techniques and methods disclosed herein can be used to practice the claimed invention as would be understood by one of ordinary skill in the art.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double-stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double-stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3- to 5′ proofreading exonuclease activity. See, for example, Vent® and Vent®. (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

Those having ordinary skill in the art, in light of this specification, will understand that many modifications, alternatives, and equivalents of the embodiments described above are possible. All such modifications, alternatives, and equivalents are intended to be encompassed herein.

### EXAMPLES

The following procedures are representative examples of embodiments according to the disclosure that may be employed for the detection of a STEC organism. These examples are not intended to be limiting to the scope of the claims and/or the disclosure in any way.

### Example 1

### Compositions & Methods to Detect STEC Microorganisms

Real-time PCR methods were developed to detect one or more E. coli strains. Some of these strains are adulterants in beef samples. The USDA has classified E. coli O157:H7 as an adulterant since 1994 and has added to the list of adulterants in 2011 several non-O157:H7 shiga-toxin producing E. coli (STEC) serotypes if they contain both stx and eae virulence factors which includes the six serotypes O26, O45, O103, O111, O121, and O145. These 6 serotypes of non-O157:H7 STEC are also referred to as the “big 6” or “top six.”

In some embodiments, several singleplex PCR assay methods were presently developed for each adulterant STEC E. coli microorganism including O26, O45, O103, O111, O121, and O145 and extensively tested for specificity and sensitivity. These singleplex assays can be combined with each other and/or with assays to detect O157:H7 and/or with assays to detect stx and/or eae in multiplex assay formats.

The present example describes methods comprising singleplex assays designed to detect STEC E. coli microorganism including serotypes O26, O45, O103, O111, O121, and O145 in a sample using probe and primer sequences designed herein. The present example also describes methods comprising singleplex assays designed to detect the presence of an eae virulence factor present in STEC E. coli adulterants in a sample using probe and primer sequences designed herein. Primer sequences comprising pairs of forward and reverse primers and corresponding probe sequences for detection of the big 6 serotypes, eae as well as stx1, stx2 and E. coli O157:H7 are shown in Table 2 below.

In one embodiment, an exemplary method of detecting the presence of a STEC organisms in a sample, may comprise: 1) separating or isolating a STEC microorganism present in a sample from other sample components; and 2) detecting the presence of at least one unique nucleic acid target region of a STEC serotype of O26, O45, O103, O111, O121, and O145 and/or fragments thereof and/or complement thereof (and in some embodiments additionally detecting the presence of target nucleic acids of virulence factors such as stx and/or eae and/or fragments thereof and/or complements thereof) using various primer and/or probe combinations described in Table 2 below.

In some embodiments, step 1) of separating or isolating a STEC microorganism present in a sample from other sample components may comprise immunomagnetic separation using magnetic beads. For example, magnetic beads coated with an antibody operable to bind to an E. coli O157:H7, an E. coli O26, an E. coli O45, an E. coli O103, an E. coli O111, an E. coli O121 and/or an E. coli O145 may be used to bind one or more of these STEC microorganisms that may be present in a sample to the magnetic beads to separate these STEC microbes from other microbes in the sample and other sample components to obtain “separated STEC microorganisms” attached to and/or bound to and/or associated with the magnetic beads. In some embodiments, magnetic beads used for immunomagnetic separation are typically PCR compatible beads and hence beads with separated STEC microorganisms can be directly used for PCR without the need for nucleic acid (RNA/DNA) extraction. In some embodiments, beads are treated in order to lyse the bead-associated bacteria, for example, by contacting the beads with a PCR-compatible lysis buffer and/or boiling the beads for 5 to 10 minutes.

In some embodiments, magnetic beads used in immunomagnetic separation may have more than one antibody attached thereon with specificity to one or more STEC microbe. In some embodiments, an immunomagnetic bead can have from one to as many as six or seven antibodies comprising for example one antibody each operable to bind to an E. coli O157:H7, an E. coli O26, an E. coli O45, an E. coli O103, an E. coli O111, an E. coli O121 and an E. coli O145.

In another embodiment, an exemplary method of detecting the presence of a STEC organisms in a sample, may comprise: 1) isolating nucleic acid from a sample and 2) detecting the presence of at least one unique nucleic acid target region of STEC serotypes O26, O45, O103, O111, O121, and O145 and/or fragments thereof and/or complement thereof (and in some embodiments optionally also detecting the presence of STEC virulence factors such as stx and/or eae and/or fragments thereof and/or complement thereof) as shown using various primer and/or probe combinations described in Table 2 below.

Various assays comprising primer probe combinations for PCR are shown in Table 2. The first column of Table 2 describes a gene target or microbe name such as stx1, stx2, eae, E. coli O121, E. coli O145, E. coli O26, E. coli O45, E. coli O103, E. coli O111, and E. coli O157:H7. The second column describes an Assay ID number (such as 14252, 14254 etc.) which is assigned to describe a primer pair and associated probe that may be used for detection of that target nucleic acid sequence. Column 3 of Table 2 describes the name of each primer and probe sequences described by the assay of column 2 (for example “14252 FOR” describes the forward primer, 14252 REV describes the reverse primer, and 14252 VIC describes a probe sequence, all three primer and probe sequences designed for Assay ID Number 14252 which is an assay to detect an stx1 specific nucleic acid). Column 4 of table 2 describes the nucleic acid sequence of the probe or primer described in column 3. These specific combinations of primer pairs and probe sequences have been designed to selectively amplify a target nucleic acid from a microbe or a virulence factor described in column 1. In some embodiments these primer pairs are degenerate.

For example, in Table 2, a forward (with suffix FOR) and reverse (with suffix REV) primer pair is shown for each target gene, which if used in an amplification reaction, will amplify the corresponding target nucleic acid specific for a virulence factor or a microbe. For example, Assay ID number 14252 describes a method for detecting the presence of an organism expressing shigatoxin (stx) having an stx1 gene comprising: contacting a sample suspected of being contaminated with a forward primer, designated in column 3 as 14252 FOR, having the nucleic acid sequence GTGACAGTAGCTATACCACGTTACA (SEQ ID NO: 1) and a reverse primer, designated as 14252 REV, having the nucleic acid sequence AGTGTTGTACGAAATCCCCTCT (SEQ ID NO: 2) under conditions to amplify a target nucleic acid sequence of an stx1 gene loci, or a fragment or a complement thereof, to form an amplified stx1 gene product. In some embodiments, detecting an amplified product, amplified using the primer pair 14252 FOR and 14252 REV can be detected using probe 14252 VIC, having the nucleic acid sequence ATGGCGATTTATCTGCATCCC (SEQ ID NO: 3).

Table 2 describes singleplex assays as outlined above with primer and probe sequences for stx1 (1 assay 14252); stx2 (3 assays 14254, 29133 and 29134) with an “Allstx2 VIC” probe described in SEQ ID NO. 10 that can detect all three of the stx2 assay amplified products; eae (1 assay 29131 comprising one forward primer and two reverse primers and a probe); E. coli O121 (1 assay 28292); E coli O145 (1 assay 28408); E. coli O26 (1 assay 28611); E. coli O45 (1 assay 28689); E. coli O103 (1 assay 28191); E. coli O111 (1 assay 28230) and E. coli O157:H7 (2 assays 18158 and 19055). Primers and probes for an internal positive control (IPC) are also used as controls (not expressly described).

TABLE 2
Assays for detecting virulence factors and STEC microorganisms:

| Gene Target or | Assay | Name of | |
|---|---|---|---|
| Microorganism | ID | Primer or | Nucleic Acid Sequence of |
| to be detected | Number | Probe | Primer or Probe |
| stx1 | 14252 | 14252 FOR | GTGACAGTAGCTATACCACGTTACA |
| | | | SEQ ID NO. 1 |
| | | 14252 REV | AGTGTTGTACGAAATCCCCTCT |
| | | | SEQ ID NO. 2 |
| | | 14252 VIC | ATGGCGATTTATCTGCATCCC |
| | | | SEQ ID NO. 3 |
| stx2 | 14254 | 14254 FOR | CGTTTTGACCATCTTCGTCTGATT |
| | | | SEQ ID NO. 4 |
| | | 14254 REV | TGCGACACGTTGCAGAGT 18 nt |
| | | | SEQ ID NO. 5 |
| | | 29133 FOR | CGTTTTGACCATCTTCGTCTGATT |
| | | | SEQ ID NO. 6 |
| | 29133 | 2913 REV | TGCGACACGTTGCAGCGT |
| | | | SEQ ID No. 7 |
| | 29134 | 29134 FOR | GATTTCTCACATATTTCAGTGCCTGATG |
| | | | SEQ ID NO. 8 |
| | | 29134 REV | CCAGATCTGCGATTCGCTGTAAT |
| | | | SEQ ID NO. 9 |
| | | Allstx2 VIC | ACGGACAGCAGTTATAC |
| | | | SEQ ID NO. 10 |
| eae | 29131 | 62396 FOR | GCGAATACTGGCGAGACTATTTCAA |
| | | | SEQ ID NO. 11 |
| | | 62396 REV | GCTCATCATAGTCTTTCTTATTGTATGACTCA |
| | | | SEQ ID NO. 12 |
| | | 62397 REV | GCTCGTCATAGTCTTTCTTGTTGTATGACTCA |
| | | | SEQ ID NO. 13 |
| | | 62396 NED-EQ | CCGCTCATGCGGAAATAGCCGTT |
| | | | SEQ ID NO. 14 |
| E. coli O121 | 28292 | 28292 FOR | TGGAAACTATCGAACAACATCAAGGT |
| | | | SEQ ID NO. 15 |
| | | 28292 REV | TGAATACTTCATCTTTGGTTAGCCATCC |
| | | | SEQ ID NO. 16 |
| | | 28292 FAM | ATTGTACAAGGCGATTTC |
| | | | SEQ ID NO. 17 |
| E. coli O145 | 28408 | 28408 FOR | ATGTTATTTGGATGCCGTTATCTCCAT |
| | | | SEQ ID NO. 18 |
| | | 28408 REV | GCTTGTAACGTGAAATGTAAACCATCA |
| | | | SEQ ID NO. 19 |
| | | 28408 FAM | CCGAACTCATCATAAAAAG |
| | | | SEQ ID NO. 20 |
| E. coli O26 | 28611 | 28611 FOR | GAAATAATTCCTTTGATCAGGGTAGAGGT |
| | | | SEQ ID NO. 21 |
| | | 28611 REV | AATATTTGAAGAAGGAAGGCATAAGGACAT |
| | | | SEQ ID NO. 22 |
| | | 28611 FAM | CAGGCTCCAATACCCATATGT |
| | | | SEQ ID NO. 23 |
| E. coli O45 | 28689 | 28689 FOR | ACAGAGTATTGGGAGAGGTTTATGAGAA |
| | | | SEQ ID NO. 24 |
| | | 28689 REV | GAAGAAAACCTACCAAGACAACAATTGAA |
| | | | SEQ ID NO. 25 |
| | | 28689 FAM | TTGAGGTTTGTTCTTTATTTCC |
| | | | SEQ ID NO. 26 |
| E. coli O103 | 28191 | 28191 FOR | CATGCGTTATAGAAATGCAATTGATAGACA |
| | | | SEQ ID NO. 27 |
| | | 28191 REV | CCACTGAACAGATGAATAACAGAACCA |
| | | | SEQ ID NO. 28 |
| | | 28191 FAM | AACGCGAATTATTTTCG |
| | | | SEQ ID NO. 29 |
| E. coli O111 | 28230 | 28230 FOR | GGCACTGATGCTAAAGCTGTAAAC |
| | | | SEQ ID NO. 30 |
| | | 28230 REV | GCAGGCCTAAAATACCTTGGATCTA |
| | | | SEQ ID NO. 31 |
| | | 28230 FAM | CCGGGCGATGTAATTA |
| | | | SEQ ID NO. 32 |
| E. coli | 18158 | 18158 FOR | CTTCCTGCAACTTGCAACTTGAA |
| O157:H7 | | | SEQ ID NO. 33 |
| | | 18158 REV | CTCGCCTGATCGACAACAAAATG |
| | | | SEQ ID NO. 34 |
| | | 18158 FAM | AGCTGGCGTAATACTTATACTCTA |
| | | | SEQ ID NO. 35 |
| | 19055 | 19055 FOR | TGACAGTATCATTACAAAGCCAAATCAGT |
| | | | SEQ ID NO. 36 |
| | | 19055 REV | GGCCACTACGCCCTTAATCTC |
| | | | SEQ ID NO. 37 |
| | | 19055 VIC | ACTGAATAAAGAGTTAAACGCC |
| | | | SEQ ID NO. 38 |

As described in Table 2, an exemplary method of detecting the presence of an eae virulence factor expressing microbe in a sample comprises hybridizing at least one pair of PCR primers such as a forward primer having nucleic acid sequences of SEQ ID NO: 11 and a reverse primer having a nucleic acid sequence of SEQ ID NO:12 and/or SEQ ID NO: 13, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an eae encoding nucleic acid in the sample. A method for detecting a microbe expressing an eae gene may further comprise using a probe to detect the at least one amplified target polynucleotide sequence, wherein the probe comprises a sequence of SEQ ID NO: 14 (to detect an eae nucleic acid product amplified by using primer pairs of SEQ ID NO. 11 and SEQ ID NO. 12 and/or SEQ ID NO. 13), fragments thereof and complements thereof and sequences having at least 90% sequence homology thereto.

Table 2 also describes an exemplary methods for detection of an E. coli O121 in a sample comprising: hybridizing at least a first pair of PCR primers comprising nucleic acids of SEQ ID NO: 15 (as a forward primer) and SEQ ID NO: 16 (as a reverse primer), fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O121 in the sample. A method to detect E. coli O121 may further comprise using a probe to detect the at least one amplified target polynucleotide sequence wherein the probe comprises a sequence of SEQ ID NO: 17, fragments having at least 10 contiguous nucleotides thereof, complements thereof, and sequences having at least 90% homology thereto.

Table 2 also describes an exemplary method for detection of an E. coli O145 in a sample comprising: hybridizing at least a first pair of PCR primers comprising nucleic acids of SEQ ID NO: 18 and SEQ ID NO: 19, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain at least one amplified target polynucleotide sequence; and detecting the at least one amplified target polynucleotide sequence; wherein detection of the at least one amplified target polynucleotide sequence is indicative of the presence of an E. coli O145 in the sample. A method to detect an E. coli O145 may further comprise using a probe to detect the at least one amplified target polynucleotide sequence wherein the probe comprises a sequence of SEQ ID NO: 20, fragments having at least 10 contiguous nucleotides thereof, complements thereof, and sequences having at least 90% homology thereto.

Table 2 also describes an exemplary method for detection of an E. coli O26 in a sample and comprise: hybridizing at least a first pair of PCR primers comprising nucleic acids of SEQ ID NO: 21 and SEQ ID NO: 22, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O26 in the sample. A method to detect an E. coli O26 may further comprise using a probe to detect the at least one amplified target polynucleotide sequence wherein the probe comprises a sequence of SEQ ID NO: 23, fragments having at least 10 contiguous nucleotides thereof, complements thereof, and sequences having at least 90% homology thereto.

Table 2 also describes an exemplary method detection of an E. coli O45 in a sample comprising: hybridizing at least a first pair of PCR primers comprising nucleic acids of SEQ ID NO: 24 and SEQ ID NO: 25, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O45 in the sample. A method to detect an E. coli O45 may further comprise using a probe to detect the at least one amplified target polynucleotide sequence wherein the probe comprises a sequence of SEQ ID NO: 26, fragments having at least 10 contiguous nucleotides thereof, complements thereof, and sequences having at least 90% homology thereto.

Table 2 also describes an exemplary method for detection of an E. coli O103 in a sample comprising: hybridizing at least a first pair of PCR primers comprising nucleic acids of SEQ ID NO: 27 and SEQ ID NO: 28, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O103 in the sample. A method to detect an E. coli O103 may further comprise using a probe to detect the at least one amplified target polynucleotide sequence wherein the probe comprises a sequence of SEQ ID NO: 29, fragments having at least 10 contiguous nucleotides thereof, complements thereof, and sequences having at least 90% homology thereto.

Table 2 also describes an exemplary method for detection of an E. coli O111 in a sample comprising: hybridizing at least a first pair of PCR primers comprising nucleic acids of SEQ ID NO: 30 and SEQ ID NO: 31, fragments having at least 10 contiguous nucleotides thereof, complements thereof, and sequences having at least 90% homology thereto, to at least a first target polynucleotide sequence present in the sample; amplifying at least the first target polynucleotide sequence or a fragment or a complement thereof to obtain an amplified target polynucleotide sequence; and detecting the at least one amplified target polynucleotide sequence; wherein detection of the at least one amplified target polynucleotide sequence is indicative of the presence of an E. coli O111 in the sample. A method to detect an E. coli O111 may further comprise using a probe to detect the at least one amplified target polynucleotide sequence wherein the probe comprises a sequence of SEQ ID NO: 32, fragments having at least 10 contiguous nucleotides thereof, complements thereof, and sequences having at least 90% homology thereto.

Table 2 also describes example primers, probes and assay methods for detecting E. coli O157:H7, including the two assays 18158 and 19055 which are also described in U.S. Provisional Patent Application Ser. No. 61/178,931, filed May 15, 2009 (Attorney Docket NO. LT00032Pro); U.S. patent application Ser. No. 12/780,707 filed May 14, 2010 (Attorney Docket NO. LT00032US); PCT Application Serial No. PCT/US2010/034998, filed May 14, 2010 (Attorney Docket NO. LT00032PCT); and European Patent Application Serial NO. 10720105.5, filed May 14, 2010, (Attorney Docket NO. LT00032EP), the entire disclosures of which are incorporated herein by reference.

Table 2 also describes example primer, probe sequences operable to bind to and/or amplify and/or detect and/or identify a stx gene in SEQ ID NO. 1-SEQ ID NO. 10. Methods of assays for stx1: Assay ID NO. 14252 and for stx2: Assay ID Nos. 14254, 29133 and 29134 are described in U.S. Provisional Patent Application Ser. No. 61/527,107, filed Aug. 24, 2011 (Attorney Docket NO. LT00569Pro) and U.S. patent application Ser. No. 13/594,682, filed Aug. 24, 2012 and PCT/US2012/052383, filed Aug. 24, 2012, the entire disclosures of which are incorporated herein by reference. Probe primers and assays for stx, including multiplex assays for detection of all stx variants are shown in Table 3 below.

TABLE 3

| | | | | *For | *Rev | |
|---|---|---|---|---|---|---|
| *Assay ID | *Forward primer | *Reverse primer | *Probe | Tm | Tm | *Prob |
| stx1 | | | | | | |
| 14252 | GTGACAGTAGCTATACCACG | AGTGTTGTACGAAATCCCC | ATGGCGATTTATCTGCATC | 61.26 | 60.73 | 71.89 |
| | TTACA (SEQ ID NO: 1) | TCT (SEQ ID NO: 2) | CC (SEQ ID NO: 3) | | | |
| stx2 | | | | | | |
| 14254 | CGTTTTGACCATCTTCGTCT | TGCGACACGTTGCAGAGT | ACGGACAGCAGTTATAC | 61.09 | 62.37 | 70.1 |
| | GATT (SEQ ID NO: 4) | (SEQ ID NO: 5) | (SEQ ID NO: 10) | | | |
| 29133 | CGTTTTGACCATCTTCGTCT | TGCGACACGTTGCAGCGT | ACGGACAGCAGTTATAC | 61.09 | 65.99 | 70.1 |
| | GATT (SEQ ID NO: 6) | (SEQ ID NO: 7) | (SEQ ID NO: 10) | | | |
| 29134 | GATTTCTCACATATTTCAGT | CCAGATCTGCGATTCGCTG | ACGGACAGCAGTTATAC | 61.64 | 62.51 | 70.1 |
| | GCCTGATG (SEQ ID NO: 8) | TAAT (SEQ ID NO: 9) | (SEQ ID NO: 10) | | | |

| | |
|---|---|
| Multiplex | Combination of assays 14254, 29133, and 29134. Detects all stx2 alleles. |
| Assay 203 | |
| stx1/stx2 | |
| Multiplex | Multiplex 203 plus assay 14252. Detects all stx1 and stx2 alleles. |
| Assay 204 | |

*Assay ID describes an assay comprising of Forward Primers and Reverse Primers described in one row and in some embodiments Probe described in the same row.
*For Tm describes the melting temperature of the Forward Primers; Rev Tm describes the melting temperature of Reverse Primers and Probe Tm describs the melting temperature of the Probe.

### Example 2

### Compositions & Methods to Detect STEC Microorganisms

In addition to the assays developed in Example 1, the present example describes additional assays developed E. coli O157:H7 as well as the non-O157:H7 shiga-toxin producing E. coli (STEC) serotypes O26, O45, O103, O111, O121, and O145.

In some embodiments, the present example describes methods comprising several singleplex PCR assay methods presently developed for each STEC E. coli microorganism including O157:H7, O26, O45, O103, O111, O121, and O145 and tested for specificity and sensitivity. Primer sequences comprising pairs of forward and reverse primers and corresponding probe sequences for detection of the big 6 serotypes and E. coli O157:H7 are shown in Table 4 below.

In one embodiment, an exemplary method of detecting the presence of a STEC organisms in a sample comprises: 1) separating or isolating a STEC microorganism present in a sample from other sample components; and 2) detecting the presence of at least one unique nucleic acid target region of a STEC serotype of O157:H7, O26, O45, O103, O111, O121, and O145 and/or fragments thereof and/or complement thereof and in some embodiments additionally detecting the presence of target nucleic acids of virulence factors such as stx and/or eae and/or fragments thereof and/or complements thereof) using various primer and/or probe combinations described in Table 2 in Example 1.

The assays described herein and in Example 1 can be combined into one or more multiplex assays to detect one or more (including all 7) STEC organisms and combined additionally with the stx and eae assays described in Example 1.

As described in Example 1, these assays can be combined with steps or methods of separating or isolating a STEC microorganism present in a sample from other sample components by immunomagnetic separation methods as described in sections above and/or by steps of 1) Lysing cells; and/or 2) isolating nucleic acid from a sample.

Various assays comprising primer probe combinations for PCR are shown in Table 4. The first column of Table 4 describes a serotype or microbe name such as E. coli O121, E. coli O145, E. coli O26, E. coli O45, E. coli O103, E. coli O111, and E. coli O157:H7. The second column describes an Assay ID number (such as 28290 etc.) which is assigned to describe a primer pair and associated probe that may be used for detection of that target nucleic acid sequence. Column 3 of Table 4 describes the target gene, column 4 describes the name of each primer and probe sequences described by the assay of column 2 (for example “fwd” describes the forward primer, “rev” describes the reverse primer, and “probe (MGB)” describes a probe sequence. All three sequences—two primer sequences and one probe sequences designed for an Assay ID Number (such as 28290) are for example an assay to detect an 0121 microorganism specific target nucleic acid). Column 5 of Table 4 describes the transcript of the target gene if known and column 6 of Table 4 describes the off-target hits (ZPCR score) which describes assay specificity.

TABLE 4
Assays for detecting virulence factors and STEC microorganisms:

| | Assay | | | | | off-target hits |
|---|---|---|---|---|---|---|
| Serotype | ID | Gene | oligo sequences | | transcript | (zpcrscore) |
| O121 | 28290 | vioA | fwd | GTCAAGAATGCCCGTGAAATAGC | dTDP-N- | S. dysenteriae |
| | | | | SEQ ID NO: 39 | acetyl- | serotype 7 (0) |
| | | | rev | GCTCAATTCTGCATGCATCGG | viosamine | |
| | | | | SEQ ID NO: 40 | synthesis | |
| | | | probe | TAGGCAAAGCACTTTCG | | |
| | | | (MGB) | SEQ ID NO: 41 | | |
| O121 | 28292 | rmlA | fwd | TGGAAACTATCGAACAACATCAAGGT | Rhamnosyl | S. dysenteriae |
| | | | | SEQ ID NO: 42 | transferase | serotype 7 (0) |
| | | | rev | TGAATACTTCATCTTTGGTTAGCCATCC | | |
| | | | | SEQ ID NO: 43 | | |
| | | | probe | ATTGTACAAGGCGATTTC | | |
| | | | (MGB) | SEQ ID NO: 44 | | |
| O121 | 28296 | wbqC | fwd | GACAGATGTGCTAGTGCAACTGATA | Unknown | S. dysenteriae |
| | | | | SEQ ID NO: 45 | function | serotype 7 (0) |
| | | | rev | GAGATTTTGTCCACCACTTGCATT | | |
| | | | | SEQ ID NO: 46 | | |
| | | | probe | CTAATTGGGATAACAAAAGC | | |
| | | | (MGB) | SEQ ID NO: 47 | | |
| O121 | 28294 | wzx | fwd | AGTGCTGGACTACAGAAAATTGCTA | O-antigen | S. dysenteriae |
| | | | | SEQ ID NO: 48 | translocase/ | serotype 7 (0) |
| | | | rev | ACCGAAATGATGGGTGCTAAGAAAA | flippase | |
| | | | | SEQ ID NO: 49 | | |
| | | | probe | CCACCAATAGTAGCGATAGTG | | |
| | | | (MGB) | SEQ ID NO: 50 | | |
| O121 | 28291 | wzx | fwd | AGAGTGATAAACTCATCTTGTCGGGTA | O-antigen | S. dysenteriae |
| | | | | T | translocase/ | serotype 7 (0) |
| | | | | SEQ ID NO: 51 | flippase | |
| | | | rev | AGAATGCCACTAGCCAACAACA | | |
| | | | | SEQ ID NO: 52 | | |
| | | | probe | CAGAAAGTGTGAAATGCC | | |
| | | | (MGB) | SEQ ID NO: 53 | | |
| O121 | 28297 | wzx | fwd | TGCGCGTTTACATGCTGAAAATAA | O-antigen | S. dysenteriae |
| | | | | SEQ ID NO: 54 | translocase/ | serotype 7 (0) |
| | | | rev | CGCTTACTCCCAAGATGCATACTAA | flippase | |
| | | | | SEQ ID NO: 55 | | |
| | | | probe | AACTTCTCCGTATTTATAAAAAT | | |
| | | | (MGB) | SEQ ID NO: 56 | | |
| O145 | 28406 | nanB | fwd | CCAGGAAACGGAATTAGTCCAATGT | neuramin- | >300 |
| | | | | SEQ ID NO: 57 | idaseB | |
| | | | rev | GAGTGCTCAATTAGTTGATCCTCATCA | | |
| | | | | SEQ ID NO: 58 | | |
| | | | probe | AATCCTTTTCAGCTATTTTAC | | |
| | | | (MGB) | SEQ ID NO: 59 | | |
| O145 | 28408 | nnaC | fwd | ATGTTATTTGGATGCCGTTATCTCCAT | CMP- | >300 |
| | | | | SEQ ID NO: 60 | NeuNAc | |
| | | | rev | GCTTGTAACGTGAAATGTAAACCATCA | synthetase | |
| | | | | SEQ ID NO: 61 | | |
| | | | probe | CCGAACGCATCATAAAAAG | | |
| | | | (MGB) | SEQ ID NO: 62 | | |
| O145 | 28413 | wckD | fwd | CCGATTACAGAGCCTTATGGATGAA | Putative | >300 |
| | | | | SEQ ID NO: 63 | Acyltrans- | |
| | | | rev | GATAGCCATTTTCCCTATATAAATGCA | ferase | |
| | | | | SEQ ID NO: 64 | | |
| | | | probe | ACCAAGCCTAGAATGTTC | | |
| | | | (MGB) | SEQ ID NO: 65 | | |
| O145 | 28412 | nnaA | fwd | CGTTATCAACTTTACCTGTTAGAGAGC | GT1 UDP- | >300 |
| | | | | A | GlcNAc 2- | |
| | | | | SEQ ID NO: 66 | Epimerase | |
| | | | rev | ACCAGTATCAGCATTTGAGCCAAT | | |
| | | | | SEQ ID NO: 67 | | |
| | | | probe | TTAGCGAAGAATACGATTATATC | | |
| | | | (MGB) | SEQ ID NO: 68 | | |
| O145 | 28414 | wzx | fwd | ACGTTTGATAGATTCGCGGTAGAAA | O-antigen | >300 |
| | | | | SEQ ID NO: 69 | translocase/ | |
| | | | rev | AGTATCCAAAAATGATTGAATAGCTGA | flippase | |
| | | | | SEQ ID NO: 70 | | |
| | | | probe | TCGGGCCTAGAAGTTT | | |
| | | | (MGB) | SEQ ID NO: 71 | | |
| O145 | 28407 | wbuW | fwd | GCTGTATCAGAGCTCGCTATCTTT | glycosyl- | >300 |
| | | | | SEQ ID NO: 72 | transferase | |
| | | | rev | GTAATCATTAGCCTGTGAGATTATATGC | | |
| | | | | TCTAA | | |
| | | | | SEQ ID NO: 73 | | |
| | | | probe | AATTCAAGCTCAATTTCG | | |
| | | | (MGB) | SEQ ID NO: 74 | | |
| O26 | 28609 | fnll- | fwd | TTTGGCGAAAGTTGCTTCTAAAACA | Epimerase/ | >300 |
| | | wbuA | | SEQ ID NO: 75 | dehydratase | |
| | | | rev | AGTACGGCATTACCAAAAGAACCA | | |
| | | | | SEQ ID NO: 76 | | |
| | | | probe | CCAGTGATAACGAGTGTTTTA | | |
| | | | (MGB) | SEQ ID NO: 77 | | |
| O26 | 28611 | wbuA | fwd | GAAATAATTCCTTTGATCAGGGTAGAG | Rhamnosyl- | >300 |
| | | | | GT | transferase | |
| | | | | SEQ ID NO: 78 | | |
| | | | rev | AATATTTGAAGAAGGAAGGCATAAGGA | | |
| | | | | CAT | | |
| | | | | SEQ ID NO: 79 | | |
| | | | probe | CAGGCTCCAATACCCATATGT | | |
| | | | (MGB) | SEQ ID NO: 80 | | |
| O26 | 28616 | wzx | fwd | GGGATATAAGGTTCAAAGTATCGCTGA | O-antigen | >300 |
| | | | | A | translocase/ | |
| | | | | SEQ ID NO: 81 | flippase | |
| | | | rev | GACAATCCAACCGAACCAAACATAA | | |
| | | | | SEQ ID NO: 82 | | |
| | | | probe | TTCATCCCTTTATCAATAATG | | |
| | | | (MGB) | SEQ ID NO: 83 | | |
| O26 | 28612 | wzx | fwd | CACTCTTGCTTCGCCTGTTG | O-antigen | >300 |
| | | | | SEQ ID NO: 84 | translocase/ | |
| | | | rev | CATGTTGCTTTATCAACCTCGTGAAA | flippase | |
| | | | | SEQ ID NO: 85 | | |
| | | | probe | TCGCGTACAGAGACGTT | | |
| | | | (MGB) | SEQ ID NO: 86 | | |
| O26 | 28617 | wzx | fwd | TTGGAGTTGGCAATAATTCAGGGT | O-antigen | >300 |
| | | | | SEQ ID NO: 87 | trnaslocase/ | |
| | | | rev | CACCAACAGCAATACTTGAAAAGATGT | flippase | |
| | | | | SEQ ID NO: 88 | | |
| | | | probe | CACAGGAAATATGGCATTCG | | |
| | | | (MGB) | SEQ ID NO: 89 | | |
| O26 | 28615 | wzx- | fwd | GGCTCATCAATTTTGAGGCTAAATGAC | dTDP-4- | >300 |
| | | rmlC | | SEQ ID NO: 90 | dehydro- | |
| | | | rev | ACCCTGAATTATTGCCAACTCCAA | rhamnose 3,5- | |
| | | | | SEQ ID NO: 91 | epimerase | |
| | | | probe | TTGATGTTGAAAAAAAAACTTC | (rmlC) | |
| | | | (MGB) | SEQ ID NO: 92 | | |
| O26 | 28614 | wzx | fwd | GCAATAATTCAGGGTGCGAATGC | O-antigen | >300 |
| | | | | SEQ ID NO: 93 | translocase/ | |
| | | | rev | CAACAGCAATACTTGAAAAGATGTTTT | flippose | |
| | | | | CC | | |
| | | | | SEQ ID NO: 94 | | |
| | | | probe | CTGTGTTGGTATTCCC | | |
| | | | (MGB) | SEQ ID NO: 95 | | |
| O26 | 28858 | wbuA | fwd | CCCAACAACTATTGGATGATTTCTTGGT | predicted | >300 |
| | | | | SEQ ID NO: 96 | rhamnosyl | |
| | | | rev | CCTTTTAGGCCAAGATTC | transferase | |
| | | | | SEQ ID NO: 97 | | |
| | | | probe | GTGAATTTGGGATATTTTGGCGGATT | | |
| | | | (MGB) | SEQ ID NO: 98 | | |
| O26 | 28861 | fnl1 | fwd | ACGGAATTATAGTCTCGCACATCAC | predicted | >300 |
| | | | | SEQ ID NO: 99 | epimerase/ | |
| | | | rev | GTAATGCCGTACTTAAGCGTTTTCT | dehydratase | |
| | | | | SEQ ID NO: 100 | | |
| | | | probe | TTTTCATCCCTGCTAAATAT | | |
| | | | (MGB) | SEQ ID NO: 101 | | |
| O26 | 28859 | wbuA | fwd | GACATAACAGGCTCCAATACCCATA | predicted | >300 |
| | | | | SEQ ID NO: 102 | rhamnosyl | |
| | | | rev | GGGAAGCAGGTTTTTAAATTATTTGAA | transferase | |
| | | | | AGG | | |
| | | | | SEQ ID NO: 103 | | |
| | | | probe | CCTGATCAAAGGAATTAT | | |
| | | | (MGB) | SEQ ID NO: 104 | | |
| O26 | 28860 | wzy | fwd | GCCATCAGCTATATATGCATGTAACGT | O-antigen | >300 |
| | | | | SEQ ID NO: 105 | polymerase | |
| | | | rev | GCGTGGGTAATAATCCAATTTCGT | | |
| | | | | SEQ ID NO: 106 | | |
| | | | probe | AAGACAGCACCAGTCCAAA | | |
| | | | (MGB) | SEQ ID NO: 107 | | |
| O26 | 28862 | wzy | fwd | CATGTAAAGCAGCAAACCAAATGC | O-antigen | >300 |
| | | | | SEQ ID NO: 108 | polymerase | |
| | | | rev | GCGGTACCCATGAAGTCATAATTG | | |
| | | | | SEQ ID NO: 109 | | |
| | | | probe | CTTGCTGGGTTTATTCC | | |
| | | | (MGB) | SEQ ID NO: 110 | | |
| O26 | 28863 | ECO26 | fwd | AGCGTCCTCAGGTCGATAGT | predicted | >300 |
| | | _3337 | | SEQ ID NO: 111 | tailspike | |
| | | | rev | CCACAGTTTGTCGATAGCACATC | protein | |
| | | | | SEQ ID NO: 112 | | |
| | | | probe | TAGCTTTTTTGAGAGAATGGTC | | |
| | | | (MGB) | SEQ ID NO: 113 | | |
| O26 | 28864 | wzx | fwd | TCTTTCCCCGCAATTTATTCAGTCT | O-antigen | >300 |
| | | | | SEQ ID NO: 114 | flippase | |
| | | | rev | GCGCTGCAATTGCTTATGTATTTG | | |
| | | | | SEQ ID NO: 115 | | |
| | | | probe | ACTGATAGTGCAAAATC | | |
| | | | (MGB) | SEQ ID NO: 116 | | |
| O26 | 28865 | ECO26 | fwd | CGGGTCATAACTACCAACGTATCC | predicted | >300 |
| | | _3337 | | SEQ ID NO: 117 | tailspike | |
| | | | rev | CGTCCAAAGTTACTGACAGGCTAT | protein | |
| | | | | SEQ ID NO: 118 | | |
| | | | probe | ACAACACAGTGAACTCC | | |
| | | | (MGB) | SEQ ID NO: 119 | | |
| O26 | 28866 | mlC | fwd | CATTTAGCCTCAAAATTGATGAGCCTTT | predicted | >300 |
| | | | | SEQ ID NO: 120 | dTDP-6- | |
| | | | rev | CAGAAGGTTGCATAAAATGGGATGA | deoxy-D- | |
| | | | | SEQ ID NO: 121 | glucose-3,5 | |
| | | | probe | TTGCTATCGGTTTATTTGGCC | epimerase | |
| | | | (MGB) | SEQ ID NO: 122 | | |
| O26 | 28867 | wzx | fwd | GGACAATGAAAACCATGAACTTTAGCT | O-antigen | >300 |
| | | | | SEQ ID NO: 123 | flippase | |
| | | | rev | CACGAGGTTGATAAAAGCAACATGAAT | | |
| | | | | SEQ ID NO: 124 | | |
| | | | probe | CTTGCATATAGCAGGCTTT | | |
| | | | (MGB) | SEQ ID NO: 125 | | |
| O45 | 28689 | wbhW | fwd | ACAGAGTATTGGGAGAGGTTTATGAGA | hypothetical | >300 |
| | | | | A | protein | |
| | | | | SEQ ID NO: 126 | | |
| | | | rev | GAAGAAAACCTACCAAGACAACAATTG | | |
| | | | | AA | | |
| | | | | SEQ ID NO: 127 | | |
| | | | probe | TTGAGGTTTGTTCTTTATTTCC | | |
| | | | (MGB) | SEQ ID NO: 128 | | |
| O45 | 28691 | wzy | fwd | GTTTATTTCCTTATGCGATTGCAATGC | O-antigen | >300 |
| | | | | SEQ ID NO: 129 | polymerase | |
| | | | rev | AAACATGAGAATGGCTCCCTGTAAA | | |
| | | | | SEQ ID NO: 130 | | |
| | | | probe | CAGTGGCTTTTATAATTTATTC | | |
| | | | (MGB) | SEQ ID NO: 131 | | |
| O45 | 28690 | wbhS | fwd | CCTGTGGCTTTTGTTGATGATAATCG | poly- | >300 |
| | | | | SEQ ID NO: 132 | saccharide | |
| | | | rev | CAACCAAGCTAATTTTTCGGGTGAA | biosynthesis | |
| | | | | SEQ ID NO: 133 | protein | |
| | | | probe | CATGGATGACCTGCCCCTG | capd | |
| | | | (MGB) | SEQ ID NO: 134 | | |
| O45 | 28695 | wbhQ | fwd | GGATGGTATTAATGGTCTTGCAAGTG | Glycosyl- | >300 |
| | | | | SEQ ID NO: 135 | transferase | |
| | | | rev | GTCATTGCATCAGATGAATCAGAGTAG | | |
| | | | | T | | |
| | | | | SEQ ID NO: 136 | | |
| | | | probe | TCGCTTTGTAGTATCTTGATAATA | | |
| | | | (MGB) | SEQ ID NO: 137 | | |
| O45 | 28696 | wbhT | fwd | CTAAGGGTGAGGATGTTGAGGTT | dTDP- | >300 |
| | | | | SEQ ID NO: 138 | glucose- | |
| | | | rev | GCAGCATAGATGACAATTCCCAGAT | 4,6- | |
| | | | | SEQ ID NO: 139 | dehydratase | |
| | | | probe | CCACCGCGTGATGAT | | |
| | | | (MGB) | SEQ ID NO: 140 | | |
| O45 | 28698 | wzx | fwd | GCAGTTGGCAATATTCTCTCGTCAT | O-antigen | >300 |
| | | | | SEQ ID NO: 141 | translocase/ | |
| | | | rev | GTTAGATGGTGCATCCAGTAATTTAGG | flippase | |
| | | | | SEQ ID NO: 142 | | |
| | | | probe | TCGGCTCCACAGATCC | | |
| | | | (MGB) | SEQ ID NO: 143 | | |
| O103 | 28196 | wzy | fwd | CTGGCTCGCGTGTGTTTT | O-antigen | >300 |
| | | | | SEQ ID NO: 144 | polymerase | |
| | | | rev | ACAAAGGCAGCCAATACTAACGT | | |
| | | | | SEQ ID NO: 145 | | |
| | | | probe | TTGAAATTTTTAATAGCAAATACCC | | |
| | | | (MGB) | SEQ ID NO: 146 | | |
| O103 | 28200 | wzy | fwd | CTGGCTCGCGTGTGTTTT | O-antigen | >300 |
| | | | | SEQ ID NO: 147 | polymerase | |
| | | | rev | ACAAAGGCAGCCAATACTAACGT | | |
| | | | | SEQ ID NO: 148 | | |
| | | | probe | ACTGCTATTCATTAAATGACAAGAC | | |
| | | | (MGB) | SEQ ID NO: 149 | | |
| O103 | 28191 | wzy | fwd | CATGCGTTATAGAAATGCAATTGATAG | O-antigen | >300 |
| | | | | ACA | polymerase | |
| | | | | SEQ ID NO: 150 | | |
| | | | rev | CCACTGAACAGATGAATAACAGAACCA | | |
| | | | | SEQ ID NO: 151 | | |
| | | | probe | AACGCGAATTATTTTCG | | |
| | | | (MGB) | SEQ ID NO: 152 | | |
| O103 | 28194 | wzy | fwd | CATGCGTTATAGAAATGCAATTGATAG | O-antigen | >300 |
| | | | | ACA | polymerase | |
| | | | | SEQ ID NO: 153 | | |
| | | | rev | CCACTGAACAGATGAATAACAGAACCA | | |
| | | | | SEQ ID NO: 154 | | |
| | | | probe | TAAATATATTAACGCGAATTATTTTC | | |
| | | | (MGB) | SEQ ID NO: 155 | | |
| O111 | 28230 | ECO11 | fwd | GGCACTGATGCTAAAGCTGTAAAC | GDP-D- | Salmonella |
| | | 1_2765 | | SEQ ID NO: 156 | mannose | Adelaide (209) |
| | | | rev | GCAGGCCTAAAATACCTTGGATCTA | dehydralase | |
| | | | | SEQ ID NO: 157 | | |
| | | | probe | CCGGGCGATGTAATTA | | |
| | | | (MGB) | SEQ ID NO: 158 | | |
| O111 | 28238 | cpsB- | fwd | AACATCGTGAATACCTTTGGCTAACT | Capsule | >300 |
| | | wbdI | | SEQ ID NO: 159 | poly- | |
| | | | rev | CCTCCGGCAATAATTACAGGGTAAA | saccharide/ | |
| | | | | SEQ ID NO: 160 | colanic | |
| | | | probe | TTCTCTCGCATATTAATTTT | acid | |
| | | | (MGB) | SEQ ID NO: 161 | synthesis | |
| O111 | 28235 | wbdM | fwd | GGCGATGGCGCATTAAGAAATAAAT | putative | Salmonella |
| | | | | SEQ ID NO: 162 | glycosyl- | Adelaide (220) |
| | | | rev | TTCTTTAATATCACTTCTTTGCCCCAAG | transferase | |
| | | | | A | | |
| | | | | SEQ ID NO: 163 | | |
| | | | probe | AATCTTGTGGATAAAGTTTTC | | |
| | | | (MGB) | SEQ ID NO: 164 | | |
| O111 | 28231 | wbdL | fwd | ACGAAGGTCCCGATAGGAACATAT | putative | Salmonella |
| | | | | SEQ ID NO: 165 | glycosyl- | Adelaide (234) |
| | | | rev | CGCTCATAATGGGCAATGGTAAATT | transferase | |
| | | | | SEQ ID NO: 166 | | |
| | | | probe | CAGGCAGTGAATGGTAC | | |
| | | | (MGB) | SEQ ID NO: 167 | | |
| O111 | 28239 | wbdL | fwd | GCTGATTGGTTTCTGAGATGTTTCA | putative | >300 |
| | | | | SEQ ID NO: 168 | glycosyl- | |
| | | | rev | GAAACCCCACCATATCCCATTCT | transferase | |
| | | | | SEQ ID NO: 169 | | |
| | | | probe | TTAATGACACGACCCCTATTGTT | | |
| | | | (MGB) | SEQ ID NO: 170 | | |
| O111 | 28234 | wbdJ | fwd | CACCTGAGTTCATAATGGGAACAAG | predicted | >300 |
| | | | | SEQ ID NO: 171 | GDP-L- | |
| | | | rev | TATTTACTTGCACTATCATGCTCTAATA | fucose | |
| | | | | T | synthetase | |
| | | | | SEQ ID NO: 172 | | |
| | | | probe | TACCAACCATGCCAGTTGAG | | |
| | | | (MGB) | SEQ ID NO: 173 | | |
| O157:H7 | 19074 | | fwd | CTTAAAATATCGCGGGCTTCGAAAT | | |
| | | | | SEQ ID NO: 174 | | |
| | | | rev | TGCCGCTGAGATATCCTAAGCT | | |
| | | | | SEQ ID NO: 175 | | |
| | | | probe | CTAGCCTCTGAAAATACATTTAT | | |
| | | | (MGB) | SEQ ID NO: 176 | | |
| O157:H7 | 19075 | | fwd | GCTGCAGTCCAGGGTACTC | | |
| | | | | SEQ ID NO: 177 | | |
| | | | rev | CCGTTCCTTAACTCAAAATGGCAAT | | |
| | | | | SEQ ID NO: 178 | | |
| | | | probe | CCCAGCATTAACTAATTCAG | | |
| | | | (MGB) | SEQ ID NO: 179 | | |
| O157:H7 | 19076 | | fwd | CGAGAAGAGATGTGTAACACGCAAT | | |
| | | | | SEQ ID NO: 180 | | |
| | | | rev | GTTTGGACAATCCATTGGTGGAATT | | |
| | | | | SEQ ID NO: 181 | | |
| | | | probe | CCACTGATCGAACCTTT | | |
| | | | (MGB) | SEQ ID NO: 182 | | |
| O157:H7 | 19077 | | fwd | GTTGTTTCTCAGCGCTGTATGTATC | | |
| | | | | SEQ ID NO: 183 | | |
| | | | rev | CGCAGTGAAAACTCTGGAGTGATTA | | |
| | | | | SEQ ID NO: 184 | | |
| | | | probe | ATCTTCATCCACTTATTATCG | | |
| | | | (MGB) | SEQ ID NO: 185 | | |
| O157:H7 | 19078 | | fwd | GTATGGCGTTGTTTTTCTCAATCAGA | | |
| | | | | SEQ ID NO: 186 | | |
| | | | rev | CCAAGAAACAGACTTCATTAGGAAGAG | | |
| | | | | T | | |
| | | | | SEQ ID NO: 187 | | |
| | | | probe | ACGGCGGTAGTTGTTAAT | | |
| | | | (MGB) | SEQ ID NO: 188 | | |
| O157:H7 | 19079 | | fwd | TCCCTTTTGGTGGTTCTGTTAGATC | | |
| | | | | SEQ ID NO: 189 | | |
| | | | rev | AGGAAGTAATACTATTTTGCGTGGTAA | | |
| | | | | AACA | | |
| | | | | SEQ ID NO: 190 | | |
| | | | probe | CAAACCCTACACCATTATC | | |
| | | | (MGB) | SEQ ID NO: 191 | | |
| O157:H7 | 19080 | | fwd | ACTAAGTCACTGCTAGAAAGATATACA | | |
| | | | | ACCA | | |
| | | | | SEQ ID NO: 192 | | |
| | | | rev | GGAAGGAACAATTACGTTGAAATGTGA | | |
| | | | | SEQ ID NO: 193 | | |
| | | | probe | CCGCGATGCTTGTTTTTTGTCG | | |
| | | | (MGB) | SEQ ID NO: 194 | | |
| O157:H7 | 19081 | | fwd | CTGTTCGCCGCAATCTTTCC | | |
| | | | | SEQ ID NO: 195 | | |
| | | | rev | CCTGTAAGCCTGAAAACACCGTTA | | |
| | | | | SEQ ID NO: 196 | | |
| | | | probe | AAGGCGATATCATCTACTTTTA | | |
| | | | (MGB) | SEQ ID NO: 197 | | |
| o157:H7 | 19082 | | fwd | GTAAATGTATCCCCCGCCAGT | | |
| | | | | SEQ ID NO: 198 | | |
| | | | rev | TGGTTGGGTATTTGTAAGGCATTGA | | |
| | | | | SEQ ID NO: 199 | | |
| | | | probe | TTGCTGGTGAGATAATTC | | |
| | | | (MGB) | SEQ ID NO: 200 | | |
| O157:H7 | 19083 | | fwd | TGCAATCACGGTATCTGAAATAAACCA | | |
| | | | | SEQ ID NO: 201 | | |
| | | | rev | GCGCAGGCAGTATTACCTTATGT | | |
| | | | | SEQ ID NO: 202 | | |
| | | | probe | TAGCCAGAGGCACACCTG | | |
| | | | (MGB) | SEQ ID NO: 203 | | |

End Table 4.

For example, Table 4 describes exemplary methods for detection of an E. coli O121 in a sample comprising: hybridizing at least one pair of PCR primers and/or probes comprising nucleic acids of SEQ ID NO: 39-SEQ ID NO: 56, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O121 in the sample. A method to detect E. coli O121 can further comprise using a probe to initially bind to a target polynucleotide sequence prior to the polymerase extending primers as in a real-time PCR method. A method to detect E. coli O121 can further comprise using a probe to detect the at least one amplified target polynucleotide sequence.

In one example embodiment a primer pair comprises a forward primer of SEQ ID NO: 39 and reverse primer of SEQ ID NO: 40 and a probe of SEQ ID NO: 41. Other examples are described in Table 4 and Table 2.

Table 4 also describes exemplary methods for detection of an E. coli O145 in a sample comprising: hybridizing at least one pair of PCR primers and/or probes comprising nucleic acids of SEQ ID NO: 57-SEQ ID NO: 74, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O145 in the sample. A method to detect E. coli O145 can further comprise using a probe to initially bind to a target polynucleotide sequence prior to the polymerase extending primers as in a real-time PCR method. A method to detect E. coli O145 can further comprise using a probe to detect the at least one amplified target polynucleotide sequence.

In one example embodiment method a primer pair comprises a forward primer of SEQ ID NO: 57 and reverse primer of SEQ ID NO: 58 and a probe of SEQ ID NO: 59. Other examples are described in Table 4 and Table 2.

Table 4 also describes exemplary methods for detection of an E. coli O26 in a sample comprising: hybridizing at least one pair of PCR primers and/or probes comprising nucleic acids of SEQ ID NO: 75-SEQ ID NO: 125, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O26 in the sample. A method to detect E. coli O26 can further comprise using a probe to initially bind to a target polynucleotide sequence prior to the polymerase extending primers as in a real-time PCR method. A method to detect E. coli O26 can further comprise using a probe to detect the at least one amplified target polynucleotide sequence.

In one example embodiment a primer pair comprises a forward primer of SEQ ID NO: 75 and reverse primer of SEQ ID NO: 76 and a probe of SEQ ID NO: 77. Other examples are described in Table 4 and Table 2.

Table 4 also describes exemplary methods for detection of an E. coli O45 in a sample comprising: hybridizing at least one pair of PCR primers and/or probes comprising nucleic acids of SEQ ID NO: 126-SEQ ID NO: 143, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O45 in the sample. A method to detect E. coli O45 can further comprise using a probe to initially bind to a target polynucleotide sequence prior to the polymerase extending primers as in a real-time PCR method. A method to detect E. coli O45 can further comprise using a probe to detect the at least one amplified target polynucleotide sequence.

In one example embodiment a primer pair comprises a forward primer of SEQ ID NO: 126 and reverse primer of SEQ ID NO: 127 and a probe of SEQ ID NO 128. Other examples are described in Table 4 and Table 2.

Table 4 also describes exemplary methods for detection of an E. coli O103 in a sample comprising: hybridizing at least one pair of PCR primers and/or probes comprising nucleic acids of SEQ ID NO: 144-SEQ ID NO: 155, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O103 in the sample. A method to detect E. coli O103 can further comprise using a probe to initially bind to a target polynucleotide sequence prior to the polymerase extending primers as in a real-time PCR method. A method to detect E. coli O103 can further comprise using a probe to detect the at least one amplified target polynucleotide sequence.

In one example embodiment a primer pair comprises a forward primer of SEQ ID NO: 144 and reverse primer of SEQ ID NO: 145 and a probe of SEQ ID NO: 146. Other examples are described in Table 4 and Table 2.

Table 4 also describes exemplary methods for detection of an E. coli O111 in a sample comprising: hybridizing at least one pair of PCR primers and/or probes comprising nucleic acids of SEQ ID NO: 156-SEQ ID NO: 173, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O111 in the sample. A method to detect E. coli O111 can further comprise using a probe to initially bind to a target polynucleotide sequence prior to the polymerase extending primers as in a real-time PCR method. A method to detect E. coli O111 can further comprise using a probe to detect the at least one amplified target polynucleotide sequence.

In one example embodiment a primer pair comprises a forward primer of SEQ ID NO: 156 and reverse primer of SEQ ID NO: 157 and a probe of SEQ ID NO: 158. Other examples are described in Table 4 and Table 2.

Table 4 also describes exemplary methods for detection of an E. coli O157:H7 in a sample comprising: hybridizing at least one pair of PCR primers and/or probes comprising nucleic acids of SEQ ID NO: 174-SEQ ID NO: 203, fragments having at least 10 contiguous nucleotides thereof, complements thereof, sequences having at least 90% homology thereto and labeled derivatives thereof, to at least a first target polynucleotide sequence present in the sample; and detecting the target nucleic acid. Primers and probes are indicated in Table 4. One or more primer pairs, indicated by a forward and a reverse primer in Table 4, can be used to hybridize specifically to a target nucleic acid in a sample and amplify at least the first (or more e.g., a second, a third . . . ) target polynucleotide sequence or a fragment or a complement thereof to obtain at least one (or more) amplified target polynucleotide sequence; and detecting the at least one (or more) amplified target polynucleotide sequence; wherein detection of the at least one amplified target polynucleotide sequence is indicative of the presence of an E. coli O157:H7 in the sample. A method to detect E. coli O157:H7 can further comprise using a probe to initially bind to a target polynucleotide sequence prior to the polymerase extending primers as in a real-time PCR method. A method to detect E. coli O157:H7 can further comprise using a probe to detect the at least one amplified target polynucleotide sequence.

In one example embodiment a primer pair comprises a forward primer of SEQ ID NO: 174 and reverse primer of SEQ ID NO: 175 and a probe of SEQ ID NO: 176. Other examples are described in Table 4 and Table 2.

The primers, probes and assay methods for detecting E. coli O157:H7 described here as assay ID No's 19074-19083 as well as the two assays 18158 and 19055 described in Example 1 (which are also described in U.S. Provisional Patent Application Ser. No. 61/178,931, filed May 15, 2009 (Attorney Docket NO. LT00032Pro); U.S. patent application Ser. No. 12/780,707 filed May 14, 2010 (Attorney Docket NO. LT00032US); PCT Application Serial No. PCT/US2010/034998, filed May 14, 2010 (Attorney Docket NO. LT00032PCT); and European Patent Application Serial NO. 10720105.5, filed May 14, 2010, (Attorney Docket NO. LT00032EP), the entire disclosures of which are incorporated herein by reference), are designed to be used as singleplex assays and/or as multiplex assays in combination with each other or with the other Big 6 assays described herein and/or with other stx and eae assays described in this specification.

### Example 3

### Specificity of Big 6 O-serotype, eae, and stx Assays: Inclusion Exclusion Data

The present example describes specificity testing performed with the assays describes in Tables 2 and Table 4 to show specific detection of a microbe or a virulence factor that the assay was designed for. The assays were tested on various species of microbes and specific detection of only microbes that the assays were designed for, i.e. detection of Big 6 pathogens and detection of microbes expressing eae and/or stx, was seen.

Stx1 inclusion testing was done using a panel of E. coli strains that contain all known stx serotypes (Table 5 below). All known stx1 serotypes were detected with a single stx assay, and all known stx2 serotypes were detected by one of three stx2 assays. When the stx1 and the three stx2 assays were combined into a single assay mixture then all stx1 and stx2 serotypes were detected in one reaction mix.

| | | | | | | | Multiplex | | |
|---|---|---|---|---|---|---|---|---|---|
| | | | Stx1 | Stx2 | Stx2 | Stx2 | Screening Assay | | |
| | | Stx | assay | assay ID | assay ID | assay | | | O157: |
| Strain | Serotype | subtype | ID 14252 | 14254 | 29134 | ID 29133 | Stx1/2 | eae | H7 |
| AA1 | O174:H8 | 1c, 2b | ++++ | ++++ | | | ++++ | +++ | |
| BB2 | O55:H7 | 1a | ++++ | | | | ++++ | ++++ | + |
| CC3 | O128ac[H2] | 2f | | | | ++++ | ++++ | ++++ | |
| DD4 | O177:[H25] | 2c, 2d | | ++++ | | | ++++ | ++++ | |
| EE5 | O111:[H8] | 1a, 2a | ++++ | ++++ | | | ++++ | ++++ | |
| FF6 | O113:H4 | 1c, 2b | ++++ | ++++ | | | ++++ | +++ | |
| GG7 | O103:H2 | 1a | ++++ | | | | ++++ | ++++ | |
| HH8 | O26:H11 | 1a | ++++ | | | | ++++ | ++++ | |
| II9 | O41:H26 | 1d | ++++ | | | | ++++ | | |
| JJ10 | O157:H7 | 2c | | ++++ | | | ++++ | ++++ | ++++ |
| O5622 | O138 | 2e | | ++++ | | | ++++ | | |
| B2F1 | O91:H21 | 2d | | ++++ | | | ++++ | | |
| D3509 | O2:H25 | 2g | | | ++++ | | ++++ | | |

This reference panel of strains contain all known stx1 and stx2 subtypes consisting of stx1a, stx1c, stx1d, stx2a, stx2b, stx2c, stx2d, stx2e, stx2f, and stx2f (P. C. H. Feng et al, (2011) Appl. Environ. Microbiol. 77:6699-6702). Each strain was tested with a singleplex real-time PCR assay and with the multiplex real-time PCR assay created by combining the assays for stx1, stx2, eae, and O157:H7 into a single reaction mix. For the multiplex real-time PCR assay, stx1 and stx2 were detected with VIC, O157:H7 was detected with FAM, and eae was detected with NED. Positive detection is indicated with a “+” and no detection is left blank.

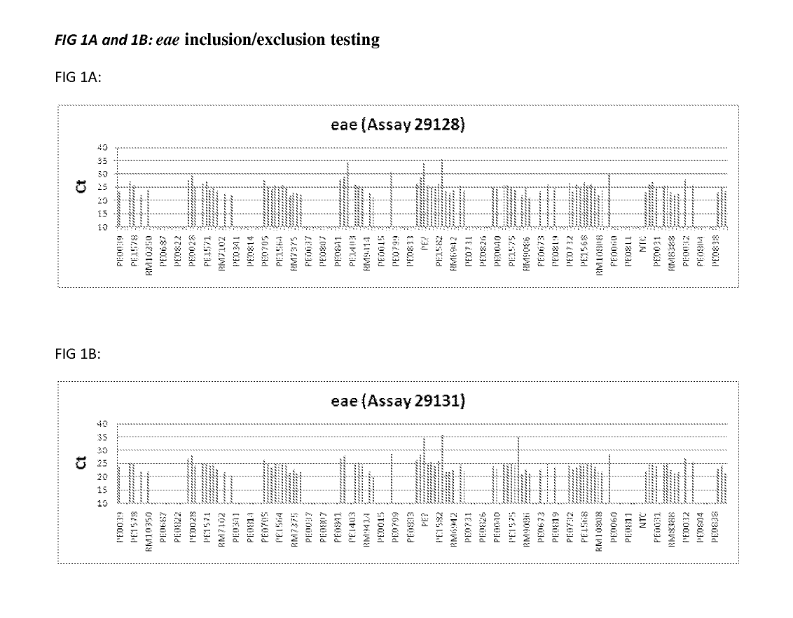

Eae inclusion and exclusion testing was done with 161 independent strains and is shown in FIG. 1A and 1B. The presence of eae in the panel of E. coli strains were not known, and therefore the ability to detect eae was determined based on the consistency of detecting eae with two independent eae assays. The results of FIG. 1A and FIG. 1B show nearly identical detection of eae in 85 of the 161 E. coli strains.

In these studies, a panel of 161 independent E. coli strains were screened for the presence of an eae gene target using two real-time PCR assays against eae. The eae genotype of the strains were not known. FIG. 1A shows the results for eae assay 29128, and FIG. 1B shows the results for eae assay 29131. The results indicate similar detection by the two independent assays. The strain designations are listed on the X axis, and positive detection is indicated by the presence of a bar on the graph above the strain (based on Ct score).

The assays for the STEC big 6 described here are unique and highly specific. Each assay described was screened against a panel of over 241 E. coli strains consisting of 167 of the 180 known E. coli O-types. The results of each assay showed 100% detection of the inclusion strains tested, and no detection of the exclusion strains tested, see Tables 6A and 6B & 7 below which depict data for inclusion/exclusion panels as a list for 157 strains:

TABLE 6A
Inclusion Panel for Set 1

| | Number of |
|---|---|
| Organism | strains |
| E. coli 0103 | 14 |
| E. coli 026 | 13 |
| E. coli 0111 | 13 |
| E. coli 045 | 11 |
| E. coli 0121 | 10 |
| E. coli 0145 | 8 |
| E. coli 0157 | 8 |

TABLE 6B
Exclusion Panel for Set 1

| | Number of |
|---|---|
| Organism | strains |
| E. coli 01 | 1 |
| E. coli 02 | 1 |
| E. coli 04 | 1 |
| E. coli 05 | 3 |
| E. coli 06 | 1 |
| E. coli 08 | 1 |
| E. coli 09 | 1 |
| E. coli 018 | 2 |
| E. coli 028 | 1 |
| E. coli 038 | 1 |
| E. coli 039 | 1 |
| E. coli 048 | 1 |
| E. coli 055 | 13 |
| E. coli 078 | 1 |
| E. coli 082 | 1 |
| E. coli 0124 | 1 |
| E. coli 0133 | 1 |
| E. coli 0134 | 1 |
| E. coli 0135 | 1 |
| E. coli 0137 | 1 |
| E. coli 0140 | 1 |
| E. coli 0141 | 1 |
| E. coli 0142 | 1 |
| E. coli 0143 | 1 |
| E. coli 0144 | 1 |
| E. coli 0146 | 1 |
| E. coli 0147 | 1 |
| E. coli 0148 | 1 |
| E. coli 0150 | 1 |
| E. coli 0151 | 1 |
| E. coli 0152 | 1 |
| E. coli 0153 | 2 |
| E. coli 0154 | 1 |
| E. coli 0155 | 1 |
| E. coli 0156 | 2 |
| E. coli 0158 | 1 |
| E. coli 0159 | 1 |
| E. coli 0160 | 1 |
| E. coli 0161 | 1 |
| E. coli 0163 | 2 |
| E. coli 0164 | 1 |
| Legionella | 1 |
| pneumoniae | |
| Listeria | 1 |
| monocytogenes | |
| Salmonella | 3 |
| Enteritidis | |
| Salmonella Typhi | 1 |
| Salmonella | 4 |
| Typhimurium | |
| Shigella | 3 |
| Vibrio cholera | 1 |

TABLE 7
Inclusion/Exclusion Strains (Panel 2) 264 strains
Inclusion Panel:

| Big 6 Organism Name | Number of strains |
|---|---|
| E. coli 026 | 9 |
| E. coli 0111 | 7 |
| E. coli 045 | 16 |
| E. coli 0121 | 7 |
| E. coli 0103 | 7 |
| E. coli 0145 | 6 |
| E. coli 0157 | 5 |

Exclusion Panel:

E. coli O groups O1-O175 (excluding O31, O47, O67, O72, O93, O94, O122)

### Example 4

### Multiple STEC Organism Detection Methods

In some embodiments, combinations of the singleplex assays described in Examples 1 and 2 above were combined into multiplex assays. Two example multiplex real-time PCR assay methods are described here which work together to initially screen a sample for the presence of an adulterant STEC organism and then to confirm the presence of an STEC adulterant in the sample.

In one example method, a first multiplex assay, also sometimes referred to as a first “screening assay” detects the presence in a sample of one or more target nucleic acid sequences of E. coli O157:H7, stx1, stx2, and eae. If a sample is positive it may have a target nucleic acid corresponding to either E. coli O157:H7 and/or stx (stx1 and/or stx2) and/or eae, or have two of the three targets, or have all the three targets. Such a positive sample is then tested again by a multiplex “confirmation assay” which is operable to detect all 6 STEC serotypes, including O26, O45, O103, O111, O121, and O145, and also operable to confirm the presence of E. coli O157:H7. The results from both assays will indicate the absence or possible presence of all E. coli STEC serotypes that have been currently identified by the USDA as adulterants.

Multiplex assays as described here allow individual detection of all nucleic acid targets of the STEC microorganisms desired to be detected within two reaction tubes by using multiple fluorophores. For example, in one example a “screening” multiplex assay detects E. coli O157:H7 with the fluorophore FAM; detects stx1 and stx2 with the fluorophore VIC; detects eae with the fluorophore LIZ; and detects an internal positive control (IPC) with the fluorophore NED. The “confirmation” multiplex assay detects E. coli O157:H7 with VIC; the big 6 non-O157:H7 STEC with FAM; and detects IPC with LIZ. The results from these two assays reveals the genotype of microorganisms within a sample matrix being tested, allowing a user (such as a food sample analyst) to decide if the sample is potentially adulterated and needs further testing.

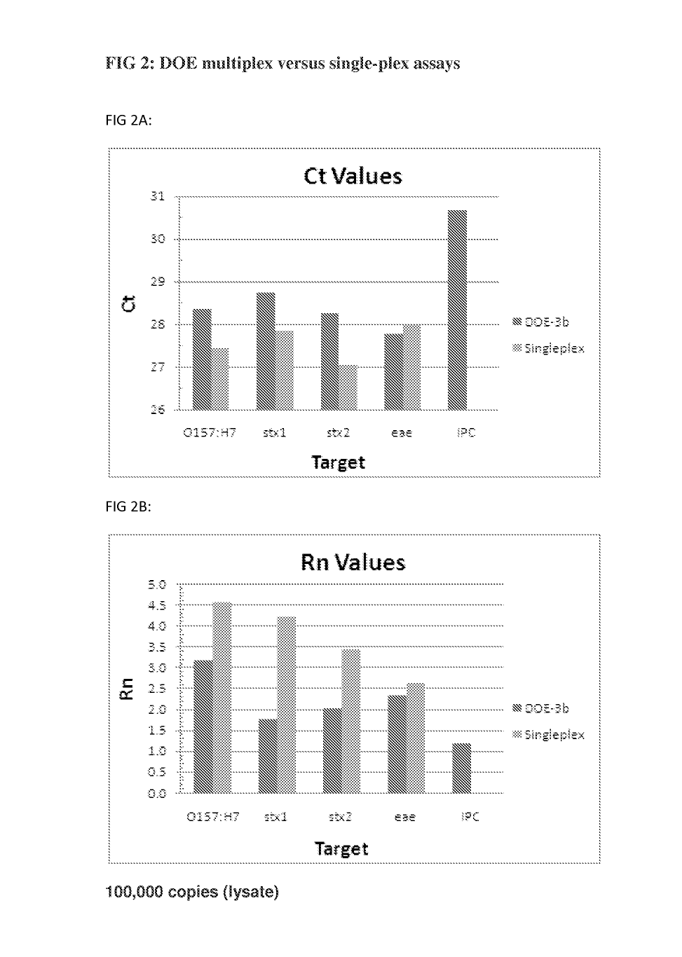

In some exemplary embodiments, TaqMan chemistry was used to design a real-time PCR method to detect E. coli O157:H7 and the “big 6” non-O157:H7 STEC serotypes. A minimum of four TaqMan® real-time PCR assays were designed against each of the 6 non-0157 STEC O-serotypes using Life Technologies proprietary assay design software. Additional assays were also designed against virulence factors stx1, stx2, and eae. Each assay was tested against an inclusion/exclusion panel of 241 E. coli strains consisting of 167 of the 180 known E. coli O-types to determine assay specificity and sensitivity (results described in Example 2). Multiple assays for each of the 6 non-O157 STECs detected all inclusion strains within the targeted serotype, and no exclusion strains from other serotypes. The stx assays detected all variants of stx1 and stx2 tested including stx2f and stx2g. The assays were combined into two separate multiplex assays and optimized using statistical methods such as Design of Experiments (DOE) (see for e.g., FIGS. 2A and 2B and also Table 5).

### Example 5

### Multiple STEC Organism Detection Workflows and Kits

The present disclosure also describes a workflow that combines the compositions and methods described herein to provide a rapid testing workflow for sample testing, such as testing complex food samples to determine the presence of adulterants. The present workflows provide a rapid time-to-result testing method for testing food samples, in less than 8 hours, rapidly to determine if food samples are contaminated with STEC organisms.

Ground beef and beef trim samples comprising 375 g of meat can be combined with 1 liter of prewarmed TSB broth (prewarmed to 48° C.) and homogenized by hand mixing. The meat samples can be incubated at 42° C. in a forced air incubator for 6.5 hours, and then the enriched meat samples can be tested for E. coli adulterants using the STEC real-time PCR screening and real-time PCR confirmation assays. The enriched samples can be prepared for real-time PCR by one of several methods. One method uses IMS capture by combining 1 mL of enriched meat sample with 10 to 30 μL of immunomagnetic beads capable of capturing E. coli strains O157:H7, O26, O45, O103, O111, O121, and O145. IMS capture can be automated using the MagMax Express-96 magnetic particle processor using a preset script to capture bacteria on the IMS beads, wash the IMS beads, and then elute the IMS beads into a PCR-compatible elution buffer. Automated capture can be completed in approximately 30 minutes. The elution sample containing IMS beads can be added directly to a real-time PCR reaction mix or heat lysed and then combined with a real-time PCR reaction mix. Real-time PCR can be performed on a 7500Fast instrument in less than 50 minutes.

A workflow method for obtaining rapid time-to result according to one example comprises: 1) Sample preparation which may comprise for example a) pooling Dynabeads for 7 pathogenic STECs. Pooling can be performed by combining Dynabeads prepared individually for each adulterant E. coli serotype into a single mixture of Dynabeads. Alternatively, Dynabeads can be prepared by linking antibodies for all adulterant E. coli serotypes on a single lot of beads in a single linking reaction. Alternatively, Dynabeads can be prepared by a combination of the two previous methods. b) 10× buffer; and c) PrepSEQ Lysis Buffer; 2) Screening Assay which may comprise for example: a) Standard lyophized bead assay consisting of primers and probe for all assays, master mix solution, and excipients required for lyophilization, b) one assay for E. coli O157:H7 (one of the two assays for O157:H7), assays for stx1/2 (capable of detecting all known variants), an assay for eae (capable of detecting all known variants) and an assay for IPC; and performing 3) a Confirmation Assay comprising: a) Standard lyophized bead assay consisting of primers and probe for all assays, master mix solution, and excipients required for lyophilization, b) one assay for E. coli O157:H7 (one of the two assays for O157:H7), assays for the Big six O types of E. coli as defined by the USDA Regulation, and an assay for IPC.

A workflow method for obtaining rapid time-to result according to another example comprises: 1) Sample preparation which comprises a nucleic acid extraction method (for example PrepSEQ™ Nucleic Acid Extraction); 2) Screening Assay which may comprise for example: a) Standard lyophized bead assay consisting of primers and probe for all assays, master mix solution, and excipients required for lyophilization, b) one assay for E. coli O157:H7 (one of the two assays for O157:H7), assays for stx1/2 (capable of detecting all known variants), an assay for eae (capable of detecting all known variants) and an assay for an internal positive control (IPC); and performing 3) a Confirmation Assay comprising: a) Standard lyophilized bead assay consisting of primers and probe for all assays, master mix solution, and excipients required for lyophilization, b) one assay for E. coli O157:H7 (one of the two assays for O157:H7), one assays each for the Big six O types of E. coli as described in Table 3 above (Big 6 are as defined by the USDA Regulation); and an assay for IPC.

A workflow for rapid time to results according to one example includes Real-time PCR detection using the Applied Biosystems 7500Fast instrument and selecting fast cycling conditions. The cycling time can include a denaturation step consisting of 95° C. for 5 minutes to lyse bacteria captured on the Dynal IMS beads prior to PCR cycling for real-time detection. Alternatively, a denaturation step of 95° C. for 2 minutes can be applied if the bacteria are prelysed. Default cycling conditions for the 7500Fast instrument (using fast cycling mode) includes a 95° C. denaturation step for 2 minutes, followed by 40 cycles consisting of a 95° C. denaturation step for 3 seconds and a 60° C. polymerization step for 30 seconds.

A workflow for rapid time to results according to one example includes a software analysis tool for interpreting real-time PCR results in order to provide a simple presence/absence call. One example of a software analysis tool is Rapid Finder Express software, or RFE. RFE software contains settings for PCR cycle number and relative fluorescence signal strength to set thresholds that must be met to determine if a sample is positive.

While the foregoing specification teaches the principles of the present invention, with examples provided for the purpose of illustration, it will be appreciated by one skilled in the art from reading this disclosure that various changes in form and detail can be made without departing from the spirit and scope of the invention. These methods are not limited to any particular type of sample or nucleic acid contained therein for e.g., total genome DNA, RNA, cDNA and the like may be analyzed using some or all of the methods disclosed in this disclosure. This disclosure provides powerful tools for analysis of complex nucleic acid samples. From experiment design to detection of shiga toxin expressing microbes, the above disclosure provides for fast, efficient and inexpensive methods for detection of pathogenic organisms.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. An isolated nucleic acid sequence comprising SEQ ID NO:1-SEQ ID NO:32 and SEQ ID NO: 39-SEQ ID NO: 203, fragments thereof, complements thereof, sequences comprising at least 90% nucleic acid sequence identity thereto and labeled derivatives thereof.

2. A method for detecting a shiga toxin producing E. Coli (STEC) microorganism in a sample comprising:
hybridizing at least a first pair of primers to a STEC target nucleic acid, a fragment thereof, a complements thereof, an allele thereof, or a variant thereof, to at least a first target polynucleotide sequence present in the sample;
amplifying at least the first target polynucleotide sequence or a fragment or a complement thereof to obtain at least one amplified target polynucleotide sequence;
detecting the at least one amplified target polynucleotide sequence; and
the method optionally further comprising using a probe to detect the at least one amplified target polynucleotide sequence,
wherein detection of the at least one amplified target polynucleotide sequence is indicative of the presence of an STEC microorganism in the sample, wherein the STEC microorganisms is an E. coli O157:H7, an E. coli O26, an E. coli O45, an E. coli O103, an E. coli O111, an E. coli O121 or an E. coli O145.

3. The method of claim 2, wherein the STEC microorganism is an E. coli O121 and wherein the at least first pair of primers comprises a primer pair selected from SEQ ID. NO: 15 and SEQ ID NO: 16, or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55, complements thereof, labeled derivatives thereof, and sequences having at least 90% homology thereto; and
wherein the probe comprises a sequence of SEQ ID NO: 17, SEQ ID NO: 41, SEQ ID NO:44, SEQ ID NO:47, SEQ ID NO:50, SEQ ID NO:53, or SEQ ID NO:56, complements thereof, labeled derivatives thereof, and sequences having at least 90% homology thereto.

4. The method of claim 2, wherein the STEC microorganism is an E. coli O145 and wherein the at least first pair of primers comprises a primer pair selected from SEQ ID NO: 18 and SEQ ID NO: 19, SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74, complements thereof, labeled derivatives thereof, and sequences having at least 90% homology thereto; and
wherein the probe comprises a sequence of SEQ ID NO: 20, SEQ ID NO: 59, SEQ ID NO:62, SEQ ID NO:65, SEQ ID NO:68, SEQ ID NO:71, or SEQ ID NO:74, complements thereof, labeled derivatives thereof, and sequences having at least 90% homology thereto.

5. The method of claim 2, wherein the STEC microorganism is an E. coli O26 and wherein the at least first pair of primers comprises a primer pair selected from SEQ ID NO: 21 and SEQ ID NO: 22, or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124, complements thereof, labeled derivatives thereof, and sequences having at least 90% homology thereto; and
wherein the probe comprises a sequence of SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, or SEQ ID NO: 125, complements thereof, labeled derivatives thereof, and sequences having at least 90% homology thereto.

6. The method of claim 2, wherein the STEC microorganism is an E. coli O45 and wherein the at least first pair of primers comprises a primer pair selected from SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142, complements thereof, labeled derivatives thereof, and sequences having at least 90% homology thereto; and
wherein the probe comprises a sequence of SEQ ID NO: 26, SEQ ID NO: 128, SEQ ID NO: 131, SEQ ID NO: 134, SEQ ID NO: 137, SEQ ID NO: 140, or SEQ ID NO: 143, complements thereof, labeled derivatives thereof, and sequences having at least 90% homology thereto.

7. The method of claim 2, wherein the STEC microorganism is an E. coli O103 and wherein the at least first pair of primers comprises a primer pair selected from SEQ ID NO: 27 and SEQ ID NO: 28, or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154, complements thereof, labeled derivatives thereof, and sequences having at least 90% homology thereto; and
wherein the probe comprises a sequence of SEQ ID NO: 29, SEQ ID NO: 146, SEQ ID NO: 149, SEQ ID NO: 152, or SEQ ID NO: 155, complements thereof, labeled derivatives thereof, and sequences having at least 90% homology thereto.

8. The method of claim 2, wherein the STEC microorganism is an E. coli O111 and wherein the at least first pair of primers comprises a primer pair selected from SEQ ID NO: 30 and SEQ ID NO: 31, or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172, complements thereof, labeled derivatives thereof, and sequences having at least 90% homology thereto; and
wherein the probe comprises a sequence of SEQ ID NO: 32, SEQ ID NO: 158, SEQ ID NO: 161, SEQ ID NO: 164, SEQ ID NO: 167, SEQ ID NO: 170, or SEQ ID NO: 173, complements thereof, labeled derivatives thereof, and sequences having at least 90% homology thereto.

9. The method of claim 2, wherein the STEC microorganism is an E. coli O157:H7 and wherein the at least first pair of primers comprises a primer pair selected from SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202 complements thereof, labeled derivatives thereof, and sequences having at least 90% homology thereto; and
wherein the probe comprises a sequence of SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200, or SEQ ID NO: 203, complements thereof, labeled derivatives thereof, and sequences having at least 90% homology thereto.

10. A method for detection of one or more microorganisms expressing at least one virulence factor comprising:
1) detecting the presence of one or more virulence factors comprising:
a) contacting a sample suspected of containing one or more microorganisms expressing at least one virulence factors with one or more sets of primers specific to the one or more virulence factors;
b) amplifying at least one target nucleic acid sequence encoding at least one of the virulence factors to obtain one or more amplified virulence factor specific nucleic acids;
c) detecting the one or more amplified virulence factor specific nucleic acids or fragments or complements thereof; and
d) optionally identifying the amplified virulence factor specific nucleic acids,
wherein detecting the one or more amplified virulence factor specific nucleic acids or fragments or complements thereof is indicative of the presence of a microorganism in the sample expressing the at least one virulence factor; and
2) determining strain of the one or more microorganism expressing the at least one virulence factor comprising:
a) contacting the sample with at least one set of primers specific to a strain of the one or more microorganism;
b) amplifying at least one strain specific target nucleic acid sequence encoding for nucleic acid targets specific to the at least one strain of the microorganism to obtain one or more amplified strain specific nucleic acids;
c) detecting the one or more amplified strain specific nucleic acid or fragments or complements thereof; and
d) optionally identifying the amplified strain specific nucleic acids.

11. The method of claim 10, wherein each set of primers specific to the one or more strain of the microorganism is labeled with a different label and wherein each set of primers specific to the one or more virulence factors is labeled with a different label.

12. The method of claim 10, wherein the steps of 1) detecting the presence of one or more virulence factors and 2) determining the strain of the one or more microorganism expressing the one or more virulence factor comprising, are carried out simultaneously or sequentially.

13. The method of claim 10, wherein the one or more sets of primers specific to the one or more virulence factors each comprise: at least a forward primer and at least a reverse primer, that are operable to hybridize to one or more virulence factors comprising all variants of shiga toxin (stx) encoding nucleic acids, alleles and fragments thereof and all variants of eae gene encoding nucleic acids, alleles and fragments thereof.

14. The method of claim 13, wherein stx encoding nucleic acids comprise an stx1 gene, a stx2 gene, an allele of an stx1 gene an allele of an stx2 gene, or a fragments thereof and wherein the eae encoding nucleic acids comprise an eae gene, an allele of an eae gene, or a fragments thereof.

15. The method of claim 10, wherein the one or more primer sets specific for the one or more virulence factors is selected from a first primer set having SEQ ID NO: 1 and SEQ ID NO 2; a second primer set having SEQ ID NO: 4 and SEQ ID NO 5; a third primer set having SEQ ID NO: 6 and SEQ ID NO:7; a fourth primer set having SEQ ID NO: 8 and SEQ ID NO: 9, a fifth primer set having SEQ ID NO: 11, SEQ ID NO: 12; and SEQ ID NO: 13.

16. The method of claim 10, wherein the one or more microorganisms is an E. coli O157:H7, an E. coli O26, an E. coli O45, an E. coli O103, an E. coli O111, an E. coli O121 or an E. coli O145.

17. The method of claim 10, wherein at least one set of primers specific to the strain of the one or more microorganism is selected from primer sets as follows:
wherein a first primer set comprises one or more primers selected from SEQ ID NO: 15 and SEQ ID NO 16; or SEQ ID NO: 39 and SEQ ID NO: 40, or SEQ ID NO: 42 and SEQ ID NO: 43, or SEQ ID NO:45 and SEQ ID NO: 46, or SEQ ID NO: 48 and SEQ ID NO: 49, or SEQ ID NO: 51 and SEQ ID NO:52, or SEQ ID NO:54 and SEQ ID NO: 55,
wherein a second primer set comprises one or more primers selected from SEQ ID NO: 18 and SEQ ID NO 19; SEQ ID. NO: 57 and SEQ ID NO: 58, or SEQ ID NO: 60 and SEQ ID NO:61, or SEQ ID NO:63 and SEQ ID NO:64, or SEQ ID NO:66 and SEQ ID NO: 67, or SEQ ID NO:69 and SEQ ID NO:70, or SEQ ID NO:72 and SEQ ID NO:74,
wherein a third primer set comprises one or more primers selected from SEQ ID NO: 21 and SEQ ID NO: 22; or SEQ ID. NO: 75 and SEQ ID NO: 76, or SEQ ID NO: 78 and SEQ ID NO: 79, or SEQ ID NO: 81 and SEQ ID NO: 82, or SEQ ID NO: 84 and SEQ ID NO: 85, or SEQ ID NO: 87 and SEQ ID NO: 88, or SEQ ID NO: 90 and SEQ ID NO: 91, or SEQ ID NO: 93 and SEQ ID NO: 94, SEQ ID. NO: 96 and SEQ ID NO: 97, or SEQ ID NO: 99 and SEQ ID NO: 100, or SEQ ID NO: 102 and SEQ ID NO: 103, or SEQ ID NO: 105 and SEQ ID NO: 106, or SEQ ID NO: 108 and SEQ ID NO: 109, or SEQ ID NO: 111 and SEQ ID NO: 112, or SEQ ID NO: 114 and SEQ ID NO: 115, SEQ ID. NO: 117 and SEQ ID NO:118, or SEQ ID NO: 120 and SEQ ID NO: 121, or SEQ ID NO: 123 and SEQ ID NO: 124,
wherein a fourth primer set comprises one or more primers selected from SEQ ID NO: 24 and SEQ ID NO: 25, or SEQ ID NO: 126 and SEQ ID NO: 127, or SEQ ID NO: 129 and SEQ ID NO: 130, or SEQ ID NO: 132 and SEQ ID NO: 133, or SEQ ID NO: 135 and SEQ ID NO: 136, or SEQ ID NO: 138 and SEQ ID NO: 139, or SEQ ID. NO: 141 and SEQ ID NO: 142,
wherein a fifth primer set comprises one or more primers selected from SEQ ID NO: 27, SEQ ID NO: 28; or SEQ ID NO: 144 and SEQ ID NO: 145, or SEQ ID NO: 147 and SEQ ID NO: 148, or SEQ ID NO: 150 and SEQ ID NO: 151, or SEQ ID NO: 153 and SEQ ID NO: 154,
wherein a sixth primer set comprises one or more primers selected from SEQ ID NO: 30 and SEQ ID NO: 31; or SEQ ID NO: 156 and SEQ ID NO: 157, or SEQ ID NO: 159 and SEQ ID NO: 160, or SEQ ID NO: 162 and SEQ ID NO: 163, or SEQ ID NO: 165 and SEQ ID NO: 166, or SEQ ID NO: 168 and SEQ ID NO: 169, or SEQ ID NO: 171 and SEQ ID NO: 172,
wherein a seventh primer set comprises one or more primers selected from SEQ ID NO: 33 and SEQ ID NO 34 or SEQ ID NO: 36 and SEQ ID NO: 37, or SEQ ID NO: 174 and SEQ ID NO: 175, or SEQ ID NO: 177 and SEQ ID NO: 178, or SEQ ID NO: 180 and SEQ ID NO: 181, or SEQ ID NO: 183 and SEQ ID NO: 184, or SEQ ID NO: 186 and SEQ ID NO: 187, or SEQ ID NO: 189 and SEQ ID NO: 190, SEQ ID. NO: 192 and SEQ ID NO: 193, or SEQ ID NO: 195 and SEQ ID NO: 196, or SEQ ID NO: 198 and SEQ ID NO: 199, or SEQ ID NO: 201 and SEQ ID NO: 202.

18. The method of claim 10, wherein the steps of detecting the one or more amplified virulence factor specific nucleic acids or fragments or complements thereof comprises:
hybridizing the one or more amplified virulence factor specific nucleic acids or fragments or complements thereof with a probe selected from SEQ ID NO: 3, SEQ ID NO: 10, or SEQ ID NO: 14; and
wherein the steps of detecting the one or more amplified strain specific nucleic acids or fragments or complements thereof comprises:
hybridizing the one or more amplified strain specific nucleic acids or fragments or complements thereof with a probe selected from SEQ ID NO: 17, SEQ ID NO: 41, SEQ ID NO:44, SEQ ID NO:47, SEQ ID NO:50, SEQ ID NO:53, SEQ ID NO:56, SEQ ID NO: 20, SEQ ID NO: 59, SEQ ID NO:62, SEQ ID NO:65, SEQ ID NO:68, SEQ ID NO:71, SEQ ID NO:74, SEQ ID NO: 23, SEQ ID NO: 77, SEQ ID NO:80, SEQ ID NO:83, SEQ ID NO: 86, SEQ ID NO: 89, SEQ ID NO:92, SEQ ID NO: 95, SEQ ID NO: 98, SEQ ID NO: 101, SEQ ID NO: 104, SEQ ID NO: 107, SEQ ID NO: 110, SEQ ID NO: 113, SEQ ID NO: 116, SEQ ID NO: 119, SEQ ID NO: 122, SEQ ID NO: 125, SEQ ID NO: 26, SEQ ID NO: 128, SEQ ID NO: 131, SEQ ID NO: 134, SEQ ID NO: 137, SEQ ID NO: 140, SEQ ID NO: 143, SEQ ID NO: 29, SEQ ID NO: 146, SEQ ID NO: 149, SEQ ID NO: 152, SEQ ID NO: 155, SEQ ID NO: 32, SEQ ID NO: 158, SEQ ID NO: 161, SEQ ID NO: 164, SEQ ID NO: 167, SEQ ID NO: 170, SEQ ID NO: 173, SEQ ID NO: 35, SEQ ID NO: 38, SEQ ID NO: 176, SEQ ID NO: 179, SEQ ID NO: 182, SEQ ID NO: 185, SEQ ID NO: 188, SEQ ID NO: 191, SEQ ID NO: 194, SEQ ID NO: 197, SEQ ID NO: 200 or SEQ ID NO: 203.

19. The method of claim 10, wherein the sample is a food sample, a beverage sample, an agricultural sample, a produce sample, an animal sample, a clinical sample, an environmental sample, a biological sample, a water sample and an air sample.

20. The method of claim 10, further comprising steps of:
immunomagnetic separation of the microorganisms expressing one or more virulence factors present in the sample from other sample components and other microbes in the sample, wherein the immunomagnetic separation comprises using magnetic beads having antibodies specific to one or more microorganisms expressing one or more virulence factors to bind the microorganisms expressing one or more virulence factors to the magnetic beads, thereby separating the microorganisms expressing one or more virulence factors from other microbes and sample components; and
optionally enrichment of the separated microorganisms expressing one or more virulence factors.

21. The method of claim 10, wherein the microorganism is one or more STEC microorganisms, the method comprising:
a) optional separation of the STEC microorganisms present in a sample from other sample components and other microbes in the sample to obtain separated STEC microorganisms;
b) optionally enriching the separated STEC microorganisms if step a) is performed or optionally enriching STEC microorganisms in a sample;
c) performing a multiplex polymerase chain reaction (PCR), on the sample, or on the separated STEC microorganisms if step a) is performed, to amplify and detect the presence of at least one amplified nucleic acids selected from:
nucleic acids encoding for a shiga toxin gene, including nucleic acids encoding stx1 and stx2 and complements and fragments thereof;
nucleic acids encoding an eae gene and complements and fragments thereof; and
nucleic acids specific for an E. coli strain O157:H7 and complements and fragments thereof, wherein the detection of at least one amplified nucleic acid listed above indicates the presence of an STEC microorganism or E. coli O157:H7 or both and wherein not detecting an amplified nucleic acid listed above is indicative of the absence of a STEC microorganism in the sample; and
d) if a nucleic acid is detected in step c) performing a second round of multiplex PCR with primers specific to amplify and detect the presence one or more nucleic acids encoding for target regions associated with one or more STEC microorganisms including an E. coli O157:H7, an E. coli O26, an E. coli O45, an E. coli O103, an E. coli O111, an E. coli O121 and an E. coli O145.
